SEQUENCE LISTING (112 sequences)

SEQ ID NO: 1 - protein, 24 aa, Severe acute respiratory syndrome-related coronavirus
PSKPSKRSFIEDLLFNKVTLADAG

SEQ ID NO: 2 - protein, 25 aa, Severe acute respiratory syndrome-related coronavirus
PSKPSKRSFIEDLLFNKVTLADAGF

SEQ ID NO: 3 - protein, 18 aa, Severe acute respiratory syndrome-related coronavirus
SFIEDLLFNKVTLADAGF

SEQ ID NO: 4 - protein, 8 aa, Severe acute respiratory syndrome-related coronavirus
SFIEDLLF

SEQ ID NO: 5 - protein, 24 aa, unidentified
  source 1..24: note = Porcine Epidemic Diarrhea Virus
GRVVQKRSFIEDLLFNKVVTNGLG

SEQ ID NO: 6 - protein, 25 aa, synthetic construct
XXXXXKRSFIEDLLFNKVXXXXXGX

SEQ ID NO: 7 - protein, 8 aa, Human immunodeficiency virus 1
AVGIGAVF

SEQ ID NO: 8 - protein, 8 aa, Human immunodeficiency virus 1
ALGIGAAF

SEQ ID NO: 9 - protein, 8 aa, Human immunodeficiency virus 1
AVGFGAAF

SEQ ID NO: 10 - protein, 8 aa, Human immunodeficiency virus 1
AAGFGAMF

SEQ ID NO: 11 - protein, 4 aa, Homo sapiens
DADA

SEQ ID NO: 12 - protein, 4 aa, Homo sapiens
EAEA

SEQ ID NO: 13 - protein, 4 aa, Homo sp.
DAEA

SEQ ID NO: 14 - protein, 4 aa, Homo sp.
EADA

SEQ ID NO: 15 - protein, 4 aa, Homo sapiens
NANA

SEQ ID NO: 16 - protein, 4 aa, Homo sp.
QAQA

SEQ ID NO: 17 - protein, 4 aa, Homo sp.
DANA

SEQ ID NO: 18 - protein, 4 aa, Homo sp.
EANA

SEQ ID NO: 19 - protein, 4 aa, Homo sp.
NADA

SEQ ID NO: 20 - protein, 4 aa, Homo sp.
NAQA

SEQ ID NO: 21 - protein, 4 aa, Homo sp.
HAHA

SEQ ID NO: 22 - protein, 4 aa, Homo sp.
RARA

SEQ ID NO: 23 - protein, 4 aa, Homo sp.
LALA

SEQ ID NO: 24 - protein, 4 aa, Homo sp.
HSHS

SEQ ID NO: 25 - protein, 4 aa, Homo sp.
HTHT

SEQ ID NO: 26 - protein, 4 aa, Homo sp.
LSLS

SEQ ID NO: 27 - protein, 4 aa, Homo sp.
LTLT

SEQ ID NO: 28 - protein, 4 aa, Homo sp.
RSRS

SEQ ID NO: 29 - protein, 11 aa, Homo sp.
RTRTPEPTIDE

SEQ ID NO: 30 - protein, 4 aa, Homo sp.
HPHP

SEQ ID NO: 31 - protein, 4 aa, Homo sp.
HGHG

SEQ ID NO: 32 - protein, 4 aa, Homo sp.
LPLP

SEQ ID NO: 33 - protein, 4 aa, Homo sp.
LGLG

SEQ ID NO: 34 - protein, 4 aa, Homo sp.
DSDS

SEQ ID NO: 35 - protein, 4 aa, Homo sp.
ESES

SEQ ID NO: 36 - protein, 4 aa, Homo sp.
DSES

SEQ ID NO: 37 - protein, 4 aa, Homo sp.
ESDS

SEQ ID NO: 38 - protein, 4 aa, Homo sp.
NSNS

SEQ ID NO: 39 - protein, 4 aa, Homo sp.
QSQS

SEQ ID NO: 40 - protein, 4 aa, Homo sp.
DSNS

SEQ ID NO: 41 - protein, 4 aa, Homo sp.
ESNS

SEQ ID NO: 42 - protein, 4 aa, Homo sp.
NSDS

SEQ ID NO: 43 - protein, 4 aa, Homo sp.
NSQS

SEQ ID NO: 44 - protein, 4 aa, Homo sp.
DTDT

SEQ ID NO: 45 - protein, 4 aa, Homo sp.
ETET

SEQ ID NO: 46 - protein, 4 aa, Homo sp.
DTET

SEQ ID NO: 47 - protein, 4 aa, Homo sp.
ETDT

SEQ ID NO: 48 - protein, 4 aa, Homo sp.
NTNT

SEQ ID NO: 49 - protein, 4 aa, Homo sp.
QTQT

SEQ ID NO: 50 - protein, 4 aa, Homo sp.
DTNT

SEQ ID NO: 51 - protein, 4 aa, Homo sp.
ETNT

SEQ ID NO: 52 - protein, 4 aa, Homo sp.
NTDT

SEQ ID NO: 53 - protein, 4 aa, Homo sp.
NTQT

SEQ ID NO: 54 - protein, 4 aa, Homo sp.
DPDP

SEQ ID NO: 55 - protein, 4 aa, Homo sp.
EPEP

SEQ ID NO: 56 - protein, 4 aa, Homo sp.
DPEP

SEQ ID NO: 57 - protein, 4 aa, Homo sp.
EPDP

SEQ ID NO: 58 - protein, 4 aa, Homo sp.
NPNP

SEQ ID NO: 59 - protein, 4 aa, Homo sp.
QPQP

SEQ ID NO: 60 - protein, 4 aa, Homo sp.
DPNP

SEQ ID NO: 61 - protein, 4 aa, Homo sp.
EPNP

SEQ ID NO: 62 - protein, 4 aa, Homo sp.
NPDP

SEQ ID NO: 63 - protein, 4 aa, Homo sp.
NPQP

SEQ ID NO: 64 - protein, 4 aa, Homo sp.
DGDG

SEQ ID NO: 65 - protein, 4 aa, Homo sp.
EGEG

SEQ ID NO: 66 - protein, 4 aa, Homo sp.
DGEG

SEQ ID NO: 67 - protein, 4 aa, Homo sp.
EGDG

SEQ ID NO: 68 - protein, 4 aa, Homo sp.
NGNG

SEQ ID NO: 69 - protein, 4 aa, Homo sp.
QGQG

SEQ ID NO: 70 - protein, 4 aa, Homo sp.
DGNG

SEQ ID NO: 71 - protein, 4 aa, Homo sp.
EGNG

SEQ ID NO: 72 - protein, 4 aa, Homo sp.
NGDG

SEQ ID NO: 73 - protein, 4 aa, Homo sp.
NGQG

SEQ ID NO: 74 - protein, 4 aa, Homo sp.
HAHS

SEQ ID NO: 75 - protein, 4 aa, Homo sp.
RARS

SEQ ID NO: 76 - protein, 4 aa, Homo sp.
LALS

SEQ ID NO: 77 - protein, 4 aa, Homo sp.
HSHT

SEQ ID NO: 78 - protein, 4 aa, Homo sp.
HTHP

SEQ ID NO: 79 - protein, 4 aa, Homo sp.
LSLT

SEQ ID NO: 80 - protein, 4 aa, Homo sp.
LTLP

SEQ ID NO: 81 - protein, 4 aa, Homo sp.
RSRT

SEQ ID NO: 82 - protein, 4 aa, Homo sp.
RTRP

SEQ ID NO: 83 - protein, 4 aa, Homo sp.
HPHG

SEQ ID NO: 84 - protein, 4 aa, Homo sp.
HGHA

SEQ ID NO: 85 - protein, 4 aa, Homo sp.
LPLA

SEQ ID NO: 86 - protein, 4 aa, Homo sp.
LGLA

SEQ ID NO: 87 - protein, 4 aa, Homo sp.
FGGG

SEQ ID NO: 88 - protein, 4 aa, Homo sp.
GGGF

SEQ ID NO: 89 - protein, 4 aa, Homo sp.
SGGG

SEQ ID NO: 90 - protein, 4 aa, Homo sp.
GGGS

SEQ ID NO: 91 - protein, 9 aa, synthetic construct
YPYDVPDYA

SEQ ID NO: 92 - DNA, 3432 bp, synthetic construct
  source 1..3432: mol_type = other DNA
ggccgccaccgctgagcaataactagcataaccccttggggcctctaaacgggtcttgaggggttttttgctgaaaggaggaactatatccgggtaacgaattcaagcttgatatcattcaggacgagcctcagactccagcgtaactggactgcaatcaactcactggctcaccttcacgggtgggcctttcttcggtagaagtcatctcatgaccaaaatcccttaacgtgagttacgcgcgcgtcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttagcccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgcgcagaaaggcccacccgaaggtgagccaggtgattacatttgggccctcattagaaaaactcatcgagcatcaaatgaaattgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgaggcgaaatacgcgatcgctgttaaaaggacaattacaaacaggaatcgagtgcaaccggcgcaggaacactgccagcgcatcaacaatattttcacctgaatcaggatattcttctaatacctggaacgctgtttttccggggatcgcagtggtgagtaaccatgcatcatcaggagtacggataaaatgcttgatggtcggaagtggcataaattccgtcagccagtttagtctgaccatctcatctgtaacatcattggcaacgctacctttgccatgtttcagaaacaactctggcgcatcgggcttcccatacaagcgatagattgtcgcacctgattgcccgacattatcgcgagcccatttatacccatataaatcagcatccatgttggaatttaatcgcggcctcgacgtttcccgttgaatatggctcatagctcctgaaaatctcgataactcaaaaaatacgcccggtagtgatcttatttcattatggtgaaagttggaacctcttacgtgccgatcaaaaagacggtcaaaagcctccggtcggaggcttttgactttctgctatggaggtcaggtatgatttaaatggtcagtattgagcgatatctagagaattcgtccaccacaattcagcaaattgtgaacatcatcacgttcatctttccctggttgccaatggcccattttcctgtcagtaacgagaaggtcgcgaattcaggcgctttttagactggtcgtaatgaaattctttttaagaggagatatacataggtctcacatgattaaattaaaatttggtgttttttttacagttttactatcttcagcatatgcacatggaacacctcaaaatattactcacggtgtgttaaccacgtcttcaccgagcaaaccgagcaaacgcagcttcatcgaggatctgctgttcaacaaggtgacgctggccgatgccggttttggtggcggcagaagacttgtgttaacaatggagatcttcgaacaatacagaccggagaacggaagttatgctaccaatatggcactggctaactcactgttcctcatggatttgaatgagcgtaagcaattcagggccatgagtgataatacacagcctgagtctgcatccgtgtggatgaagatcactggaggaataagctctggtaagcttaatgacgggcaaaataaaacaacaaccaatcagtttatcaatcagctcgggggggatatttataaattccatgctgaacaactgggtgattttaccttagggattatgggaggatacgcgaatgcaaaaggtaaaacgataaattacacgagcaacaaagctgccagaaacacactggatggttattctgtcggggtatacggtacgtggtatcagaatggggaaaatgcaacagggctctttgctgaaacttggatgcaatataactggtttaatgcatcagtgaaaggtgacggactggaagaagaaaaatataatctgaatggtttaaccgcttctgcaggtgggggatataacctgaatgtgcacacatggacatcacctgaaggaataacaggtgaattctggttacagcctcatttgcaggctgtctggatgggggttacaccggatacacatcaggaggataacggaacggtggtgcagggagcagggaaaaataatattcagacaaaagcaggtattcgtgcatcctggaaggtgaaaagcaccctggataaggataccgggcggaggttccgtccgtatatagaggcaaactggatccataacactcatgaatttggtgttaaaatgagtgatgacagccagttgttgtcaggtagccgaaatcagggagagataaagacaggtattgaaggggtgattactcaaaacttgtcagtgaatggcggagtcgcatatcaggcaggaggtcacgggagcaatgccatctccggagcactggggataaaatacagcttcggttagagc

SEQ ID NO: 93 - DNA, 3600 bp, synthetic construct
  source 1..3600: mol_type = other DNA
aaccacgtcttcaccgagtaaaccgagtaaacgcagctttattgaggatctgctcttcaacaaggtgacgctggccgacgccggttttggcggcggcccgagcaagccgagcaaacgtagctttatcgaggatctgctctttaataaagtgacgctggcggatgcgggtttcggcggtggcccgagcaaaccaagcaaacgtagtttcatcgaggatctgctgttcaataaagttacgctggccgatgcgggttttggtggtggtagaagacttgtgttaacaatggagatcttcgaacaatacagaccggagaacggaagttatgctaccaatatggcactggctaactcactgttcctcatggatttgaatgagcgtaagcaattcagggccatgagtgataatacacagcctgagtctgcatccgtgtggatgaagatcactggaggaataagctctggtaagcttaatgacgggcaaaataaaacaacaaccaatcagtttatcaatcagctcgggggggatatttataaattccatgctgaacaactgggtgattttaccttagggattatgggaggatacgcgaatgcaaaaggtaaaacgataaattacacgagcaacaaagctgccagaaacacactggatggttattctgtcggggtatacggtacgtggtatcagaatggggaaaatgcaacagggctctttgctgaaacttggatgcaatataactggtttaatgcatcagtgaaaggtgacggactggaagaagaaaaatataatctgaatggtttaaccgcttctgcaggtgggggatataacctgaatgtgcacacatggacatcacctgaaggaataacaggtgaattctggttacagcctcatttgcaggctgtctggatgggggttacaccggatacacatcaggaggataacggaacggtggtgcagggagcagggaaaaataatattcagacaaaagcaggtattcgtgcatcctggaaggtgaaaagcaccctggataaggataccgggcggaggttccgtccgtatatagaggcaaactggatccataacactcatgaatttggtgttaaaatgagtgatgacagccagttgttgtcaggtagccgaaatcagggagagataaagacaggtattgaaggggtgattactcaaaacttgtcagtgaatggcggagtcgcatatcaggcaggaggtcacgggagcaatgccatctccggagcactggggataaaatacagcttcggttagagcggccgccaccgctgagcaataactagcataaccccttggggcctctaaacgggtcttgaggggttttttgctgaaaggaggaactatatccgggtaacgaattcaagcttgatatcattcaggacgagcctcagactccagcgtaactggactgcaatcaactcactggctcaccttcacgggtgggcctttcttcggtagaagtcatctcatgaccaaaatcccttaacgtgagttacgcgcgcgtcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttagcccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgcgcagaaaggcccacccgaaggtgagccaggtgattacatttgggccctcattagaaaaactcatcgagcatcaaatgaaattgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgaggcgaaatacgcgatcgctgttaaaaggacaattacaaacaggaatcgagtgcaaccggcgcaggaacactgccagcgcatcaacaatattttcacctgaatcaggatattcttctaatacctggaacgctgtttttccggggatcgcagtggtgagtaaccatgcatcatcaggagtacggataaaatgcttgatggtcggaagtggcataaattccgtcagccagtttagtctgaccatctcatctgtaacatcattggcaacgctacctttgccatgtttcagaaacaactctggcgcatcgggcttcccatacaagcgatagattgtcgcacctgattgcccgacattatcgcgagcccatttatacccatataaatcagcatccatgttggaatttaatcgcggcctcgacgtttcccgttgaatatggctcatagctcctgaaaatctcgataactcaaaaaatacgcccggtagtgatcttatttcattatggtgaaagttggaacctcttacgtgccgatcaaaaagacggtcaaaagcctccggtcggaggcttttgactttctgctatggaggtcaggtatgatttaaatggtcagtattgagcgatatctagagaattcgtccaccacaattcagcaaattgtgaacatcatcacgttcatctttccctggttgccaatggcccattttcctgtcagtaacgagaaggtcgcgaattcaggcgctttttagactggtcgtaatgaaattctttttaagaggagatatacataggtctcacatgattaaattaaaatttggtgttttttttacagttttactatcttcagcatatgcacatggaacacctcaaaatattactcacggtgtgtt

SEQ ID NO: 94 - DNA, 3768 bp, synthetic construct
  source 1..3768: mol_type = other DNA
ggccgccaccgctgagcaataactagcataaccccttggggcctctaaacgggtcttgaggggttttttgctgaaaggaggaactatatccgggtaacgaattcaagcttgatatcattcaggacgagcctcagactccagcgtaactggactgcaatcaactcactggctcaccttcacgggtgggcctttcttcggtagaagtcatctcatgaccaaaatcccttaacgtgagttacgcgcgcgtcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttagcccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgcgcagaaaggcccacccgaaggtgagccaggtgattacatttgggccctcattagaaaaactcatcgagcatcaaatgaaattgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgaggcgaaatacgcgatcgctgttaaaaggacaattacaaacaggaatcgagtgcaaccggcgcaggaacactgccagcgcatcaacaatattttcacctgaatcaggatattcttctaatacctggaacgctgtttttccggggatcgcagtggtgagtaaccatgcatcatcaggagtacggataaaatgcttgatggtcggaagtggcataaattccgtcagccagtttagtctgaccatctcatctgtaacatcattggcaacgctacctttgccatgtttcagaaacaactctggcgcatcgggcttcccatacaagcgatagattgtcgcacctgattgcccgacattatcgcgagcccatttatacccatataaatcagcatccatgttggaatttaatcgcggcctcgacgtttcccgttgaatatggctcatagctcctgaaaatctcgataactcaaaaaatacgcccggtagtgatcttatttcattatggtgaaagttggaacctcttacgtgccgatcaaaaagacggtcaaaagcctccggtcggaggcttttgactttctgctatggaggtcaggtatgatttaaatggtcagtattgagcgatatctagagaattcgtccaccacaattcagcaaattgtgaacatcatcacgttcatctttccctggttgccaatggcccattttcctgtcagtaacgagaaggtcgcgaattcaggcgctttttagactggtcgtaatgaaattctttttaagaggagatatacataggtctcacatgattaaattaaaatttggtgttttttttacagttttactatcttcagcatatgcacatggaacacctcaaaatattactcacggtgtgttaaccacgtcttcaccgagtaaaccgagtaaacgcagctttattgaggatctgctcttcaacaaggtgacgctggccgacgccggttttggcggcggcccgagcaagccgagcaaacgtagctttatcgaggatctgctctttaataaagtgacgctggcggatgcgggtttcggcggtggcccgagcaaaccaagcaaacgtagtttcatcgaggatctgctgttcaataaagttacgctggccgatgcgggttttggtggtggtccaagcaaaccaagtaagcgtagttttattgaggatctgctgtttaacaaggttaccctcgccgatgccggttttggtggcggtccgagtaagccgagtaagcgtagcttcattgaagatctgctgttcaacaaagtgacgctggccgatgccggctttggcggtggtagaagacttgtgttaacaatggagatcttcgaacaatacagaccggagaacggaagttatgctaccaatatggcactggctaactcactgttcctcatggatttgaatgagcgtaagcaattcagggccatgagtgataatacacagcctgagtctgcatccgtgtggatgaagatcactggaggaataagctctggtaagcttaatgacgggcaaaataaaacaacaaccaatcagtttatcaatcagctcgggggggatatttataaattccatgctgaacaactgggtgattttaccttagggattatgggaggatacgcgaatgcaaaaggtaaaacgataaattacacgagcaacaaagctgccagaaacacactggatggttattctgtcggggtatacggtacgtggtatcagaatggggaaaatgcaacagggctctttgctgaaacttggatgcaatataactggtttaatgcatcagtgaaaggtgacggactggaagaagaaaaatataatctgaatggtttaaccgcttctgcaggtgggggatataacctgaatgtgcacacatggacatcacctgaaggaataacaggtgaattctggttacagcctcatttgcaggctgtctggatgggggttacaccggatacacatcaggaggataacggaacggtggtgcagggagcagggaaaaataatattcagacaaaagcaggtattcgtgcatcctggaaggtgaaaagcaccctggataaggataccgggcggaggttccgtccgtatatagaggcaaactggatccataacactcatgaatttggtgttaaaatgagtgatgacagccagttgttgtcaggtagccgaaatcagggagagataaagacaggtattgaaggggtgattactcaaaacttgtcagtgaatggcggagtcgcatatcaggcaggaggtcacgggagcaatgccatctccggagcactggggataaaatacagcttcggttagagc

SEQ ID NO: 95 - protein, 28 aa, synthetic construct
PSKPSKRSFIEDLLFNKVTLADAGFGGG

SEQ ID NO: 96 - protein, 84 aa, synthetic construct
PSKPSKRSFIEDLLFNKVTLADAGFGGGPSKPSKRSFIEDLLFNKVTLADAGFGGGPSKPSKRSFIEDLLFNKVTLADAGFGGG

SEQ ID NO: 97 - protein, 140 aa, synthetic construct
PSKPSKRSFIEDLLFNKVTLADAGFGGGPSKPSKRSFIEDLLFNKVTLADAGFGGGPSKPSKRSFIEDLLFNKVTLADAGFGGGPSKPSKRSFIEDLLFNKVTLADAGFGGGPSKPSKRSFIEDLLFNKVTLADAGFGGG

SEQ ID NO: 98 - DNA, 3780 bp, synthetic construct
  source 1..3780: mol_type = other DNA
aaccacgtcttcagatgccgacgcgccgagtaaaccgagtaaacgcagctttattgaggatctgctcttcaacaaggtgacgctggccgacgccggttttggcggcggcccgagcaagccgagcaaacgtagctttatcgaggatctgctctttaataaagtgacgctggcggatgcgggtttcggcggtggcccgagcaaaccaagcaaacgtagtttcatcgaggatctgctgttcaataaagttacgctggccgatgcgggttttggtggtggtccaagcaaaccaagtaagcgtagttttattgaggatctgctgtttaacaaggttaccctcgccgatgccggttttggtggcggtccgagtaagccgagtaagcgtagcttcattgaagatctgctgttcaacaaagtgacgctggccgatgccggctttggcggtggtagaagacttgtgttaacaatggagatcttcgaacaatacagaccggagaacggaagttatgctaccaatatggcactggctaactcactgttcctcatggatttgaatgagcgtaagcaattcagggccatgagtgataatacacagcctgagtctgcatccgtgtggatgaagatcactggaggaataagctctggtaagcttaatgacgggcaaaataaaacaacaaccaatcagtttatcaatcagctcgggggggatatttataaattccatgctgaacaactgggtgattttaccttagggattatgggaggatacgcgaatgcaaaaggtaaaacgataaattacacgagcaacaaagctgccagaaacacactggatggttattctgtcggggtatacggtacgtggtatcagaatggggaaaatgcaacagggctctttgctgaaacttggatgcaatataactggtttaatgcatcagtgaaaggtgacggactggaagaagaaaaatataatctgaatggtttaaccgcttctgcaggtgggggatataacctgaatgtgcacacatggacatcacctgaaggaataacaggtgaattctggttacagcctcatttgcaggctgtctggatgggggttacaccggatacacatcaggaggataacggaacggtggtgcagggagcagggaaaaataatattcagacaaaagcaggtattcgtgcatcctggaaggtgaaaagcaccctggataaggataccgggcggaggttccgtccgtatatagaggcaaactggatccataacactcatgaatttggtgttaaaatgagtgatgacagccagttgttgtcaggtagccgaaatcagggagagataaagacaggtattgaaggggtgattactcaaaacttgtcagtgaatggcggagtcgcatatcaggcaggaggtcacgggagcaatgccatctccggagcactggggataaaatacagcttcggttagagcggccgccaccgctgagcaataactagcataaccccttggggcctctaaacgggtcttgaggggttttttgctgaaaggaggaactatatccgggtaacgaattcaagcttgatatcattcaggacgagcctcagactccagcgtaactggactgcaatcaactcactggctcaccttcacgggtgggcctttcttcggtagaagtcatctcatgaccaaaatcccttaacgtgagttacgcgcgcgtcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttagcccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgcgcagaaaggcccacccgaaggtgagccaggtgattacatttgggccctcattagaaaaactcatcgagcatcaaatgaaattgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgaggcgaaatacgcgatcgctgttaaaaggacaattacaaacaggaatcgagtgcaaccggcgcaggaacactgccagcgcatcaacaatattttcacctgaatcaggatattcttctaatacctggaacgctgtttttccggggatcgcagtggtgagtaaccatgcatcatcaggagtacggataaaatgcttgatggtcggaagtggcataaattccgtcagccagtttagtctgaccatctcatctgtaacatcattggcaacgctacctttgccatgtttcagaaacaactctggcgcatcgggcttcccatacaagcgatagattgtcgcacctgattgcccgacattatcgcgagcccatttatacccatataaatcagcatccatgttggaatttaatcgcggcctcgacgtttcccgttgaatatggctcatagctcctgaaaatctcgataactcaaaaaatacgcccggtagtgatcttatttcattatggtgaaagttggaacctcttacgtgccgatcaaaaagacggtcaaaagcctccggtcggaggcttttgactttctgctatggaggtcaggtatgatttaaatggtcagtattgagcgatatctagagaattcgtccaccacaattcagcaaattgtgaacatcatcacgttcatctttccctggttgccaatggcccattttcctgtcagtaacgagaaggtcgcgaattcaggcgctttttagactggtcgtaatgaaattctttttaagaggagatatacataggtctcacatgattaaattaaaatttggtgttttttttacagttttactatcttcagcatatgcacatggaacacctcaaaatattactcacggtgtgtt

SEQ ID NO: 99 - protein, 144 aa, synthetic construct
DADAPSKPSKRSFIEDLLFNKVTLADAGFGGGPSKPSKRSFIEDLLFNKVTLADAGFGGGPSKPSKRSFIEDLLFNKVTLADAGFGGGPSKPSKRSFIEDLLFNKVTLADAGFGGGPSKPSKRSFIEDLLFNKVTLADAGFGGG

SEQ ID NO: 100 - protein, 326 aa, synthetic construct
QYRPENGSYATNMALANSLFLMDLNERKQFRAMSDNTQPESASVWMKITGGISSGKLNDGQNKTTTNQFINQLGGDIYKFHAEQLGDFTLGIMGGYANAKGKTINYTSNKAARNTLDGYSVGVYGTWYQNGENATGLFAETWMQYNWFNASVKGDGLEEEKYNLNGLTASAGGGYNLNVHTWTSPEGITGEFWLQPHLQAVWMGVTPDTHQEDNGTVVQGAGKNNIQTKAGIRASWKVKSTLDKDTGRRFRPYIEANWIHNTHEFGVKMSDDSQLLSGSRNQGEIKTGIEGVITQNLSVNGGVAYQAGGHGSNAISGALGIKYSFG

SEQ ID NO: 101 - protein, 5 aa, synthetic construct
GDGDG

SEQ ID NO: 102 - protein, 5 aa, synthetic construct
GSGSG

SEQ ID NO: 103 - protein, 5 aa, synthetic construct
GKGKG

SEQ ID NO: 104 - protein, 5 aa, synthetic construct
GDGKG

SEQ ID NO: 105 - protein, 6 aa, synthetic construct
DADADA

SEQ ID NO: 106 - protein, 8 aa, synthetic construct
DADADADA

SEQ ID NO: 107 - protein, 4 aa, synthetic construct
SASA

SEQ ID NO: 108 - protein, 8 aa, synthetic construct
SASASASA

SEQ ID NO: 109 - protein, 4 aa, synthetic construct
KAKA

SEQ ID NO: 110 - protein, 8 aa, synthetic construct
KAKAKAKA

SEQ ID NO: 111 - protein, 4 aa, synthetic construct
DAKA

SEQ ID NO: 112 - protein, 8 aa, synthetic construct
DAKADAKA

(19) United States
(12) Patent Application Publication
(10) Pub. No.: US 2025/0332246 A1
(43) Pub. Date: Oct. 30, 2025

(54) ENHANCED EXPRESSION VIA AUTOTRANSPORTERS

(71) Applicant: University of Virginia Patent Foundation, Charlottesville, VA (US)

(72) Inventor: Steven L. Zeichner, Bethesda, MD (US)

(73) Assignee: University of Virginia Patent Foundation, Charlottesville, VA (US)

(21) Appl. No.: 18/707,809

(22) PCT Filed: Nov. 4, 2022
(86) PCT No.: PCT/US2022/079351

§ 371 Date: May 6, 2024

Related U.S. Application Data
(60) Provisional application No. 63/275,802, filed on Nov. 4, 2021.

(51) Int. Cl. A61K 39/215; A61K 9/00; A61K 39/00; A61K 39/21; A61P 31/14; C07K 14/005; C07K 14/195; C12N 7/00; C12N 15/67; C12N 15/70

(52) U.S. Cl. CPC A61K 39/215; A61K 9/0043; A61K 39/21; A61P 31/14; C07K 14/005; C07K 14/195; C12N 7/00; C12N 15/67; C12N 15/70; A61K 2039/521; A61K 2039/522; A61K 2039/523; A61K 2039/543; C07K 2319/00; C12N 2740/16022; C12N 2740/16034; C12N 2770/20022; C12N 2770/20034; C12N 2800/22

(57) ABSTRACT

Provided are expression cassettes that include coding sequences encoding at least one polar amino acid or at least one expression enhancement peptide that includes one or more polar amino acids, a polypeptide of interest, and a bacterial autotransporter β-barrel polypeptide. In some embodiments, the coding sequences are in frame with each other such that transcription and translation of the expression cassette in a host cell produces a fusion protein with the polar amino acid and/or the expression enhancement peptide, the polypeptide of interest, and the bacterial autotransporter β-barrel polypeptide expressed on the surface of the host cell. Also provided are expression vectors and host cells that include the expression cassettes, vaccine compositions based on the presently disclosed compositions, and methods for inducing immune responses, enhancing expression of the polypeptides of interest, for preventing or treating viral infections such as but not limited to coronavirus infections.

### CROSS REFERENCE TO RELATED APPLICATION

This application is a U.S. National Phase Application of PCT International Patent Application Serial No. PCT/US2022/079351, filed Nov. 4, 2022, herein incorporated by reference in its entirety, which claims the benefit of and priority to U.S. Provisional patent application Ser. No. 63/275,802, filed Nov. 4, 2021, the disclosure of which is also incorporated herein by reference in its entirety.

### REFERENCE TO SEQUENCE LISTING XML

The Sequence Listing XML associated with the instant disclosure has been electronically submitted to the United States Patent and Trademark Office via the Patent Center as a 111.114 byte UTF-8-encoded XML file created on Nov. 4, 2022 and entitled “3062_175_PCT.xml”. The Sequence Listing submitted via Patent Center is hereby incorporated by reference in its entirety.

A Replacement Sequence Listing XML was filed with the United States Patent and Trademark Office via the Patent Center as a 112.277 byte UTF-8-encoded XML file created on Mar. 6, 2025 and entitled “Updated_3062_175_PCT.xml”. The Replacement Sequence Listing XML submitted via Patent Center is hereby incorporated by reference in its entirety.

### TECHNICAL FIELD

The presently disclosed subject matter relates to compositions and methods useful for inducing cellular and humoral immune responses, such as but not limited to compositions and methods employed in the context of vaccine development and antibody production. In representative embodiments, the presently disclosed subject matter relates to bacteria modified to have reduced expression of genes, such as by having a reduction of the bacterial genomes, and using those bacteria to express viral (e.g., coronavirus and/or HIV) antigens of interest. The presently disclosed subject matter also relates in some embodiments to vaccine compositions and materials to elicit useful immune responses from humans and animals comprising modified bacteria expressing antigens that induce immune responses against viruses with class I fusion proteins such as SARS-CoV-2.

### BACKGROUND

Autotransporters offer a useful and effective way of expressing recombinant proteins on the surfaces of bacterial cells. The recombinant proteins can serve a variety of functions, but a very important use is as antigens for vaccines. Some vaccines, for example subunit vaccines, can include substantial amounts of hydrophobic amino acids which may be hard to express alone. Expression on the surfaces of bacteria using the autotransporters may be particularly helpful in expressing recombinant hydrophobic proteins or peptides, because expressing such proteins or peptides in the cytoplasm of cells can yield insoluble aggregates or inclusion bodies. Because, for autotransporters, translation and export to the surface are very tightly coupled, surface expression via autotransporters offers a way to produce substantial amounts of recombinant protein without the production of the insoluble aggregates or inclusion bodies. However, expression on the surface of the bacteria of longer hydrophobic proteins may be problematic because the proteins may not remain extended out into the aqueous environment. Here it is shown in some embodiments that adding additional highly polar amino acids, by way of example aspartates, at the end of the recombinant protein substantially enhances expression of a protein of interest, such as a recombinant hydrophobic passenger protein, such multimers of the fusion peptide of the SARS-CoV-2 spike protein.

### SUMMARY

This summary lists several embodiments of the presently disclosed subject matter, and in many cases lists variations and permutations of these embodiments. This summary is merely exemplary of the numerous and varied embodiments. Mention of one or more representative features of a given embodiment is likewise exemplary. Such an embodiment can typically exist with or without the feature(s) mentioned: likewise, those features can be applied to other embodiments of the presently disclosed subject matter, whether listed in this summary or not. To avoid excessive repetition, this Summary does not list or suggest all possible combinations of such features.

In some embodiments, the presently disclosed subject matter provides an expression cassette comprising a first coding sequence encoding at least one polar amino acid and/or at least one expression enhancement peptide, a second coding sequence encoding a polypeptide of interest, and a third coding sequence encoding a bacterial autotransporter β-barrel polypeptide, wherein:
(i) the first, second, and third coding sequences are in frame with each other such that transcription and translation of the expression cassette in a host cell produces a fusion protein comprising the expression enhancement peptide, the polypeptide of interest, and the bacterial autotransporter β-barrel polypeptide;
(ii) the expression enhancement peptide comprises one or more copies of the amino acid sequence (X¹X³X³)ₙ, wherein each X¹, X², X³ is independently selected from the group consisting of Asp (D), Asn (N), Glu (E), Gln (Q), His (H), Arg (R), Lys (K); Ala (A), Ser(S), Thr (T), Pro (P), Gly (G), Met (M), Leu (L), Ile (I), Val (V), and Cys (C); each X³ is absent in any given member of the one or more copies of the amino acid sequence (X¹X²X³)ₙ or, if present, is independently any amino acid; and n is at least 1; and
(iii) when expressed in a host cell, optionally a bacterium, further optionally a Gram-negative bacterium, the expression cassette induces expression of the polypeptide of interest on a surface of the host cell.

In some embodiments, the polypeptide of interest is a concatemer of two, three, four, five, or more copies of an amino acid sequence of interest, optionally wherein each of the two, three, four, five, or more copies of the amino acid sequence of interest are linked to each other via a peptide linker.

In some embodiments, wherein the polypeptide of interest is a concatemer of two, three, four, five, or more copies of an amino acid sequence of interest, optionally wherein each of the two, three, four, five, or more copies of the amino acid sequence of interest are linked to each other via a peptide linker.

In some embodiments, the peptide linker comprises a peptide sequence selected from the group consisting of FGGG (SEQ ID NO: 87), GGGF (SEQ ID NO: 88), SGGG (SEQ ID NO: 89). GGGS (SEQ ID NO: 90), and any combination thereof.

In some embodiments, wherein at least one of the two or more copies of the amino acid sequence of interest in the concatemer comprises an expression enhancement peptide at its N-terminus, optionally wherein each of the two or more copies of the amino acid sequence of interest in the concatemer comprises an expression enhancement peptide at its N-terminus.

In some embodiments, wherein the expression enhancement peptide comprises an amino acid sequence selected from the group consisting of DA, DS, DT, DP, DG, NA, NS, NT, NP, NG, EA, ES, ET, EP, QA, QG, QA, QS, QT, QP, QG, HA, HS, HT, HP, HG, RA, RS, RT, RP, RG, LA, LS, LT, LP, LG, DAX⁴, DSX⁴, DTX⁴, DPX⁴, DGX⁴, NAX⁴, NSX⁴, NTX⁴, NPX⁴, NGX⁴, EAX⁴, ESX⁴, ETX¹, EPX⁴, QAX⁴, QGX⁴, QAX⁴, QSX⁴, QTX⁴, QPX⁴, QGX⁴, HAX⁴, HSX⁴, HTX⁴, HPX⁴, HGX⁴, RAX⁴, RSX⁴, RTX⁴, RPX⁴, RGX⁴, LAX⁴, LSX⁴, LTX⁴, LPX⁴, LGX⁴, DADA (SEQ ID NO: 11), EAEA (SEQ ID NO: 12), DAEA (SEQ ID NO: 13), EADA (SEQ ID NO: 14), NANA (SEQ ID NO: 15), QAQA (SEQ ID NO: 16), DANA (SEQ ID NO: 17), EANA (SEQ ID NO: 18), NADA (SEQ ID NO: 19), NAQA (SEQ ID NO: 20), HAHA (SEQ ID NO: 21), RARA (SEQ ID NO: 22), LALA (SEQ ID NO: 23), HSHS (SEQ ID NO: 24), HTHT (SEQ ID NO: 25), LSLS (SEQ ID NO: 26), LTLT (SEQ ID NO: 27), RSRS (SEQ ID NO: 28), RTRT (SEQ ID NO: 29), HPHP (SEQ ID NO: 30), HGHG (SEQ ID NO: 31), LPLP (SEQ ID NO: 32), LGLG (SEQ ID NO: 33), DSDS (SEQ ID NO: 34), ESES (SEQ ID NO: 35), DSES (SEQ ID NO: 36), ESDS (SEQ ID NO: 37), NSNS (SEQ ID NO: 38), QSQS (SEQ ID NO: 39), DSNS (SEQ ID NO: 40), ESNS (SEQ ID NO: 41), NSDS (SEQ ID NO: 42), NSQS (SEQ ID NO: 43), DTDT (SEQ ID NO: 44), ETET (SEQ ID NO: 45), DTET (SEQ ID NO: 46), ETDT (SEQ ID NO: 47), NTNT (SEQ ID NO: 48), QTQT (SEQ ID NO: 49), DTNT (SEQ ID NO: 50), ETNT (SEQ ID NO: 51), NTDT (SEQ ID NO: 52), NTQT (SEQ ID NO: 53), DPDP (SEQ ID NO: 54), EPEP (SEQ ID NO: 55), DPEP (SEQ ID NO: 56), EPDP (SEQ ID NO: 57), NPNP (SEQ ID NO: 58), QPQP (SEQ ID NO: 59), DPNP (SEQ ID NO: 60), EPNP (SEQ ID NO: 61), NPDP (SEQ ID NO: 62), NPQP (SEQ ID NO: 63), DGDG (SEQ ID NO: 64), EGEG (SEQ ID NO: 65), DGEG (SEQ ID NO: 66), EGDG (SEQ ID NO: 67), NGNG (SEQ ID NO: 68), QGQG (SEQ ID NO: 69), DGNG (SEQ ID NO: 70), EGNG (SEQ ID NO: 71), NGDG (SEQ ID NO: 72), NGQG (SEQ ID NO: 73), HAHS (SEQ ID NO: 74), RARS (SEQ ID NO: 75), LALS (SEQ ID NO: 76), HSHT (SEQ ID NO: 77), HTHP (SEQ ID NO: 78), LSLT (SEQ ID NO: 79), LTLP (SEQ ID NO: 80), RSRT (SEQ ID NO: 81), RTRP (SEQ ID NO: 82), HPHG (SEQ ID NO: 83), HGHA (SEQ ID NO: 84), LPLA (SEQ ID NO: 85), LGLA (SEQ ID NO: 86), and combinations thereof, wherein X⁴ is any amino acid.

In some embodiments, the polypeptide of interest is an antigen, which in some embodiments is an antigen designed to elicit an immune response. In some embodiments, the antigen is an antigen from a pathogen. In some embodiments, the antigen is an antigen from a virus. In some embodiments, the polypeptide of interest comprises an antigen from SARS-CoV-2, HIV, PEDV, HIV, or other virus. In some embodiments, wherein the viral antigen comprises a coronavirus antigen, optionally a severe acute respiratory syndrome-associated coronavirus (SARS-CoV) antigen, a severe acute respiratory syndrome-associated coronavirus 2 (SARS-CoV-2) antigen, and/or a porcine epidemic diarrhea virus (PEDV) antigen; or a human immunodeficiency virus (HIV) antigen.

In some embodiments, the viral antigen comprises:
(i) a coronavirus fusion peptide (FP) antigen, optionally a coronavirus FP antigen comprising an amino acid sequence selected from the group consisting of PSKPSKRSFIEDLLFNKVTLADAGF (SEQ ID NO: 2), PSKPSKRSFIEDLLFNKVTLADAG (SEQ ID NO: 1), SFIEDLLF (SEQ ID NO: 4), SFIEDLLFNKVTLADAGF (SEQ ID NO: 3), GRVVQKRSFIEDLLFNKVVTNGLG (SEQ ID NO: 5), and immunogenic fragments thereof; or
(ii) an HIV antigen comprising an amino acid sequence selected from the group consisting of AVGIGAVF (SEQ ID NO: 7), ALGIGAAF (SEQ ID NO: 8), AVGFGAAF (SEQ ID NO: 9), AAGFGAMF (SEQ ID NO: 10), and immunogenic fragments thereof; or,
(iii) any combination thereof.

In some embodiments, the antigen is an antigen from a biologically active molecule. In some embodiments, the antigen is an antigen from a molecule involved in an autoimmune process.

In some embodiments, the polypeptide of interest comprises an antigen against which is it desired to elicit an immune response to develop humor or cellular immunity. In some embodiments, the polypeptide of interest comprises an antigen against which is it desired to elicit a humoral and/or a cellular immune responses, which in some embodiments can be employed to develop custom antibodies or custom cell mediated immune responses.

In some embodiments, the polypeptide of interest comprises an antigen derived from and/or expressed by a tumor cell or a cancer cell.

In some embodiments, the expression vector is configured to express the polypeptide of interest in a genome reduced bacterium or derivative thereof, optionally on a surface of the genome reduced bacterium or derivative thereof.

In some embodiments, one or more of the first, second, and third coding sequences comprises a codon optimized coding sequence.

In some embodiments the presently disclosed subject matter provides an expression vector comprising the expression cassette as disclosed herein.

In some embodiments, the expression cassette is operably linked to an inducible promoter or a constitutive promoter.

In some embodiments, the expression vector further comprising a pharmaceutically acceptable carrier, excipient, or diluent.

In some embodiments the presently disclosed subject matter provides a host cell comprising the expression cassette as disclosed herein and/or the expression vector as disclosed herein.

In some embodiments, the host cell is a bacterium, optionally a Gram negative bacterium, further optionally an Escherichia coli or a Salmonella bacterium.

In some embodiments, the bacterium is a genome reduced bacterium.

In some embodiments, the genome reduced bacterium is characterized by a reduced number of expressed genes comprises a reduction of expressed genes of at least about 1.0%, 2.0%, 3.0%, 4.0%, 5.0%, 6.0%, 7.0%, 8.0%, 9.0%, 10%, 11%, 12%, 13%, 14%, 15%, or greater than 15% of genes relative to a non-genome reduced bacterium upon which the genome reduced bacterium or derivative thereof is based.

In some embodiments, the reduced number of expressed genes comprises a reduction of expressed genes selected from the group consisting of at least about 2.4%, at least about 15.9%, and at least about 29.7% relative to a non-genome reduced bacterium upon which the genome reduced bacterium or derivative thereof is based.

In some embodiments, the polypeptide of interest is expressed on a surface of the host cell.

In some embodiments the presently disclosed subject matter provides a vaccine composition comprising the expression cassette as disclosed herein, the expression vector as disclosed herein, the host cell as disclosed herein, and a pharmaceutically acceptable carrier, diluent, or excipient, one or more adjuvants, or any combination thereof.

In some embodiments, the vaccine composition comprises a bacterium, optionally a genome reduced bacterium, and further wherein the bacterium is a live attenuated bacterium or a killed whole cell bacterium or an immunogenic fragment thereof that comprises the polypeptide of interest.

In some embodiments, the vaccine composition is formulated to be administered orally, rectally, vaginally, intra-nasally, parenterally, intradermally, subcutaneously, or intramuscularly. In some embodiments, the vaccine further comprises an adjuvant.

In some embodiments, the polypeptide of interest is an antigen, optionally a viral antigen.

In some embodiments, the viral antigen comprises a coronavirus antigen, optionally a severe acute respiratory syndrome-associated coronavirus (SARS-CoV) antigen, a severe acute respiratory syndrome-associated coronavirus 2 (SARS-CoV-2) antigen, and/or a porcine epidemic diarrhea virus (PEDV) antigen; or a human immunodeficiency virus (HIV) antigen.

In some embodiments, the viral antigen comprises:
(i) a coronavirus fusion peptide (FP) antigen, optionally a coronavirus FP antigen comprising an amino acid sequence selected from the group consisting of PSKPSKRSFIEDLLFNKVTLADAGF (SEQ ID NO: 2), PSKPSKRSFIEDLLFNKVTLADAG (SEQ ID NO: 1), SFIEDLLF (SEQ ID NO: 4), SFIEDLLFNKVTLADAGF (SEQ ID NO: 3), GRVVQKRSFIEDLLFNKVVTNGLG (SEQ ID NO: 5), and immunogenic fragments thereof; or
(ii) an HIV antigen comprising an amino acid sequence selected from the group consisting of AVGIGAVF (SEQ ID NO: 7), ALGIGAAF (SEQ ID NO: 8), AVGFGAAF (SEQ ID NO: 9), AAGFGAMF (SEQ ID NO: 10), and immunogenic fragments thereof; or
(iii) any combination thereof.

In some embodiments, the presently disclosed subject matter provides a method for inducing an immune response in a subject, the method comprising administering to the subject a composition comprising, consisting essentially of, or consisting of the expression cassette as disclosed herein, the expression vector as disclosed herein, and/or the host cell as disclosed herein in an amount and via a route sufficient to induce an immune response to the polypeptide of interest in the subject.

In some embodiments, the immune response to the polypeptide of interest induced in the subject is enhanced relative to an immune response that would be induced in the subject when an expression cassette that is identical to the expression cassette as disclosed herein, an expression vector that is identical to the expression vector as disclosed herein, and/or a host cell that is identical to the host cell as disclosed herein but that lacks the first coding sequence that encodes the expression enhancement peptide is administered to the subject in the same amount and via the same route.

In some embodiments, the composition is administered parenterally.

In some embodiments, the composition is administered non-parenterally. In some embodiments, the presently disclosed subject matter provides a method for enhancing expression of a polypeptide of interest in a host cell, the method comprising modifying the polypeptide of interest to include an expression enhancement peptide moiety at or near its N-terminus, wherein the expression enhancement peptide comprises one or more copies of the amino acid sequence (X¹X²X³)ₙ, wherein each X¹, X², X³ is independently selected from the group consisting of Asp (D), Asn (N), Glu (E), Gln (Q), His (H), Arg (R). Lys (K); Ala (A), Ser(S), Thr (T), Pro (P), Gly (G). Met (M), Leu (L), Ile (I), Val (V), and Cys (C); each X³ is absent in any given member of the one or more copies of the amino acid sequence (X¹X²X³)ₙ, or, if present, is independently any amino acid; and n is at least 1.

In some embodiments, the expression enhancement peptide comprises an amino acid sequence selected from the group consisting of DA, DS, DT, DP, DG, NA, NS, NT, NP, NG, EA, ES, ET, EP, QA, QG, QA, QS, QT, QP, QG, HA, HS, HT, HP, HG, RA, RS, RT, RP, RG, LA, LS, LT, LP, LG, DAX⁴, DSX⁴, DTX⁴, DPX⁴, DGX⁴, NAX⁴, NSX⁴, NTX⁴, NPX⁴, NGX⁴, EAX⁴, ESX⁴, ETX⁴, EPX⁴, QAX⁴, QGX⁴, QAX⁴, QSX⁴, QTX⁴, QPX⁴, QGX⁴, HAX⁴, HSX⁴, HTX⁴, HPX⁴, HGX⁴, RAX⁴, RSX⁴, RTX⁴, RPX⁴, RGX⁴, LAX⁴, LSX⁴, LTX⁴, LPX⁴, LGX⁴, DADA (SEQ ID NO: 11), EAEA (SEQ ID NO: 12), DAEA (SEQ ID NO: 13), EADA (SEQ ID NO: 14), NANA (SEQ ID NO: 15), QAQA (SEQ ID NO: 16), DANA (SEQ ID NO: 17), EANA (SEQ ID NO: 18), NADA (SEQ ID NO: 19), NAQA (SEQ ID NO: 20), HAHA (SEQ ID NO: 21), RARA (SEQ ID NO: 22), LALA (SEQ ID NO: 23), HSHS (SEQ ID NO: 24), HTHT (SEQ ID NO: 25), LSLS (SEQ ID NO: 26), LTLT (SEQ ID NO: 27), RSRS (SEQ ID NO: 28), RTRT (SEQ ID NO: 29), HPHP (SEQ ID NO: 30), HGHG (SEQ ID NO: 31), LPLP (SEQ ID NO: 32), LGLG (SEQ ID NO: 33), DSDS (SEQ ID NO: 34), ESES (SEQ ID NO: 35), DSES (SEQ ID NO: 36), ESDS (SEQ ID NO: 37), NSNS (SEQ ID NO: 38), QSQS (SEQ ID NO: 39), DSNS (SEQ ID NO: 40), ESNS (SEQ ID NO: 41), NSDS (SEQ ID NO: 42), NSQS (SEQ ID NO: 43), DTDT (SEQ ID NO: 44), ETET (SEQ ID NO: 45), DTET (SEQ ID NO: 46), ETDT (SEQ ID NO: 47), NTNT (SEQ ID NO: 48), QTQT (SEQ ID NO: 49), DTNT (SEQ ID NO: 50), ETNT (SEQ ID NO: 51), NTDT (SEQ ID NO: 52), NTQT (SEQ ID NO: 53), DPDP (SEQ ID NO: 54), EPEP (SEQ ID NO: 55), DPEP (SEQ ID NO: 56), EPDP (SEQ ID NO: 57), NPNP (SEQ ID NO: 58), QPQP (SEQ ID NO: 59), DPNP (SEQ ID NO: 60), EPNP (SEQ ID NO: 61), NPDP (SEQ ID NO: 62), NPQP (SEQ ID NO: 63), DGDG (SEQ ID NO: 64), EGEG (SEQ ID NO: 65), DGEG (SEQ ID NO: 66), EGDG (SEQ ID NO: 67), NGNG (SEQ ID NO: 68), QGQG (SEQ ID NO: 69), DGNG (SEQ ID NO: 70), EGNG (SEQ ID NO: 71), NGDG (SEQ ID NO: 72), NGQG (SEQ ID NO: 73), HAHS (SEQ ID NO: 74), RARS (SEQ ID NO: 75), LALS (SEQ ID NO: 76), HSHT (SEQ ID NO: 77), HTHP (SEQ ID NO: 78), LSLT (SEQ ID NO: 79), LTLP (SEQ ID NO: 80), RSRT (SEQ ID NO: 81), RTRP (SEQ ID NO: 82), HPHG (SEQ ID NO: 83), HGHA (SEQ ID NO: 84), LPLA (SEQ ID NO: 85), LGLA (SEQ ID NO: 86), and combinations thereof, wherein X⁴ is any amino acid.

In some embodiments, the presently disclosed subject matter provides a method for preventing or treating a coronavirus infection in a subject in need thereof, the method comprising administering a vaccine composition according to any one of claims 20-26 to the subject in an amount and via a route sufficient to induce an anti-coronavirus immune response in the subject.

In some embodiments, the presently disclosed subject matter provides a method for treating an infection, optionally a viral infection, optionally a coronavirus infection or an HIV infection, further optionally a SARS-CoV infection, a SARS-CoV-2 infection, and/or a porcine epidemic diarrhea virus (PEDV) infection, in a subject in need thereof, the method comprising administering to the subject a vaccine composition according to the presently disclosed subject matter in an amount and via a route sufficient to induce an anti-infection, e.g., an anti-viral immune response in the subject.

In some embodiments of the method, the vaccine composition is administered orally, rectally, vaginally, intra-nasally, parenterally, intradermally, subcutaneously, or intramuscularly.

In some embodiments of the method, the vaccine composition further comprises a pharmaceutically acceptable carrier, excipient, and/or diluent, optionally wherein the pharmaceutically acceptable carrier, excipient, and/or diluent is pharmaceutically acceptable for use in a human.

In some embodiments of the method, the vaccine composition comprises and/or encodes:
(i) a coronavirus fusion peptide (FP) antigen, optionally a coronavirus FP antigen comprising an amino acid sequence selected from the group consisting of PSKPSKRSFIEDLLFNKVTLADAGF (SEQ ID NO: 2), PSKPSKRSFIEDLLFNKVTLADAG (SEQ ID NO: 1), SFIEDLLF (SEQ ID NO: 4), SFIEDLLFNKVTLADAGF (SEQ ID NO: 3), GRVVQKRSFIEDLLFNKVVTNGLG (SEQ ID NO: 5), and immunogenic fragments thereof; or
(ii) an HIV antigen comprising an amino acid sequence selected from the group consisting of AVGIGAVF (SEQ ID NO: 7), ALGIGAAF (SEQ ID NO: 8), AVGFGAAF (SEQ ID NO: 9), AAGFGAMF (SEQ ID NO: 10), and immunogenic fragments thereof; or
(iii) any combination thereof.

In some embodiments, the presently disclosed subject matter provides a composition comprising a modified bacterium or derivative thereof that expresses one or more tandem copies of a polypeptide of interest fused to a bacterial autotransporter β-barrel polypeptide, wherein:
(i) at least one copy of the one or more tandem copies of the polypeptide of interest is modified at or near its N-terminus to comprise an expression peptide comprising, consisting essentially of, or consisting of one or more copies of the amino acid sequence (X¹X²X³)ₙ, wherein each X¹ is independently selected from the group consisting of Asp (D), Asn (N), Glu (E), Gln (Q), His (H), Arg (R), and Lys (K); each X² is independently selected from the group consisting of Ala (A), Ser(S), Thr (T), Pro (P), Gly (G), Met (M), Leu (L), Ile (I), Val (V), and Cys (C); each X³ is absent in any given member of the one or more copies of the amino acid sequence (X¹X²X³)ₙ or, if present, is independently any amino acid; and n is at least 1; and
(ii) the polypeptide of interest is expressed on a surface of the modified bacterium or the derivative thereof.

In some embodiments, the presently disclosed subject matter provides a composition for use in treating a patient infected with a virus, optionally a coronavirus, further optionally a SARS-CoV, SARS-CoV-2, and/or porcine epidemic diarrhea virus (PEDV), wherein the composition comprises a bacterium or derivative thereof, optionally a genome reduced derivative thereof, that expresses the expression cassette as disclosed herein on its surface, wherein polypeptide of interest in the expression cassette comprises a viral antigen modified to comprise an expression enhancement peptide.

In some embodiments of the composition for use, the viral antigen is a coronavirus antigen or an HIV antigen, optionally a SARS-CoV, SARS-CoV-2, and/or porcine epidemic diarrhea virus (PEDV) antigen, further optionally wherein the viral antigen comprises:
(i) a coronavirus fusion peptide (FP) antigen, optionally a coronavirus FP antigen comprising an amino acid sequence selected from the group consisting of PSKPSKRSFIEDLLFNKVTLADAGF (SEQ ID NO: 2), PSKPSKRSFIEDLLFNKVTLADAG (SEQ ID NO: 1), SFIEDLLF (SEQ ID NO: 4), SFIEDLLFNKVTLADAGF (SEQ ID NO: 3), GRVVQKRSFIEDLLFNKVVTNGLG (SEQ ID NO: 5), and immunogenic fragments thereof; or
(ii) an HIV antigen comprising an amino acid sequence selected from the group consisting of AVGIGAVF (SEQ ID NO: 7), ALGIGAAF (SEQ ID NO: 8), AVGFGAAF (SEQ ID NO: 9), AAGFGAMF (SEQ ID NO: 10), and immunogenic fragments thereof; or
(iii) any combination thereof.

In some embodiments, the presently disclosed subject matter provides a composition for use in treating a subject infected with a virus, optionally a coronavirus or an HIV virus, further optionally a SARS-CoV virus, a SARS-CoV-2 virus, and/or porcine epidemic diarrhea virus (PEDV), wherein the composition comprises a bacterium or a genome reduced derivative thereof that expresses a protein comprising a viral antigen on its surface, wherein the protein comprises one or more tandem copies of the viral antigen at least one of which has been modified to comprise an expression enhancement peptide at or near its N-terminus.

In some embodiments of the composition for use, the expression enhancement peptide comprises, consists essentially of, or consists of one or more copies of the amino acid sequence (X¹X²X³)ₙ, wherein each X¹, X², X³ is independently selected from the group consisting of Asp (D). Asn (N), Glu (E), Gln (Q), His (H), Arg (R), Lys (K); Ala (A), Ser(S), Thr (T), Pro (P), Gly (G), Met (M), Leu (L), Ile (I), Val (V), and Cys (C); each X³ is absent in any given member of the one or more copies of the amino acid sequence (X¹X²X³)ₙ or, if present, is independently any amino acid; and n is at least 1.

In some embodiments of the composition for use, the expression enhancement peptide comprises an amino acid sequence selected from the group consisting of DA, DS, DT, DP, DG, NA, NS, NT, NP, NG, EA, ES, ET, EP, QA, QG, QA, QS, QT, QP, QG, HA, HS, HT, HP, HG, RA, RS, RT, RP, RG, LA, LS, LT, LP, LG, DAX⁴, DSX⁴, DTX⁴, DPX⁴, DGX⁴, NAX⁴, NSX⁴, NTX⁴, NPX⁴, NGX⁴, EAX⁴, ESX⁴, ETX⁴, EPX⁴, QAX⁴, QGX⁴, QAX⁴, QSX⁴, QTX⁴, QPX⁴, QGX⁴, HAX⁴, HSX⁴, HTX⁴, HPX⁴, HGX⁴, RAX⁴, RSX⁴, RTX⁴, RPX⁴, RGX⁴, LAX⁴, LSX⁴, LTX⁴, LPX⁴, LGX⁴, DADA (SEQ ID NO: 11), EAEA (SEQ ID NO: 12), DAEA (SEQ ID NO: 13), EADA (SEQ ID NO: 14), NANA (SEQ ID NO: 15), QAQA (SEQ ID NO: 16), DANA (SEQ ID NO: 17), EANA (SEQ ID NO: 18), NADA (SEQ ID NO: 19), NAQA (SEQ ID NO: 20), HAHA (SEQ ID NO: 21), RARA (SEQ ID NO: 22), LALA (SEQ ID NO: 23), HSHS (SEQ ID NO: 24), HTHT (SEQ ID NO: 25), LSLS (SEQ ID NO: 26), LTLT (SEQ ID NO: 27), RSRS (SEQ ID NO: 28), RTRT (SEQ ID NO: 29), HPHP (SEQ ID NO: 30), HGHG (SEQ ID NO: 31), LPLP (SEQ ID NO: 32), LGLG (SEQ ID NO: 33), DSDS (SEQ ID NO: 34), ESES (SEQ ID NO: 35), DSES (SEQ ID NO: 36), ESDS (SEQ ID NO: 37), NSNS (SEQ ID NO: 38), QSQS (SEQ ID NO: 39), DSNS (SEQ ID NO: 40), ESNS (SEQ ID NO: 41), NSDS (SEQ ID NO: 42), NSQS (SEQ ID NO: 43), DTDT (SEQ ID NO: 44), ETET (SEQ ID NO: 45), DTET (SEQ ID NO: 46), ETDT (SEQ ID NO: 47), NTNT (SEQ ID NO: 48), QTQT (SEQ ID NO: 49), DTNT (SEQ ID NO: 50), ETNT (SEQ ID NO: 51), NTDT (SEQ ID NO: 52), NTQT (SEQ ID NO: 53), DPDP (SEQ ID NO: 54), EPEP (SEQ ID NO: 55), DPEP (SEQ ID NO: 56), EPDP (SEQ ID NO: 57), NPNP (SEQ ID NO: 58), QPQP (SEQ ID NO: 59), DPNP (SEQ ID NO: 60), EPNP (SEQ ID NO: 61). NPDP (SEQ ID NO: 62), NPQP (SEQ ID NO: 63), DGDG (SEQ ID NO: 64), EGEG (SEQ ID NO: 65), DGEG (SEQ ID NO: 66), EGDG (SEQ ID NO: 67), NGNG (SEQ ID NO: 68), QGQG (SEQ ID NO: 69), DGNG (SEQ ID NO: 70), EGNG (SEQ ID NO: 71), NGDG (SEQ ID NO: 72), NGQG (SEQ ID NO: 73), HAHS (SEQ ID NO: 74), RARS (SEQ ID NO: 75), LALS (SEQ ID NO: 76). HSHT (SEQ ID NO: 77), HTHP (SEQ ID NO: 78), LSLT (SEQ ID NO: 79), LTLP (SEQ ID NO: 80), RSRT (SEQ ID NO: 81), RTRP (SEQ ID NO: 82), HPHG (SEQ ID NO: 83), HGHA (SEQ ID NO: 84), LPLA (SEQ ID NO: 85), LGLA (SEQ ID NO: 86), and combinations thereof, wherein X⁴ is any amino acid.

In some embodiments of the composition for use, the viral antigen comprises a coronavirus antigen, optionally wherein the viral antigen comprises a SARS-CoV antigen, a SARS-CoV-2 antigen, or a porcine epidemic diarrhea virus (PEDV) antigen; or an HIV virus.

In some embodiments of the composition for use, the viral antigen comprises:
(i) a coronavirus fusion peptide (FP) antigen, optionally a coronavirus FP antigen comprising an amino acid sequence selected from the group consisting of PSKPSKRSFIEDLLFNKVTLADAGF (SEQ ID NO: 2), PSKPSKRSFIEDLLFNKVTLADAG (SEQ ID NO: 1), SFIEDLLF (SEQ ID NO: 4), SFIEDLLFNKVTLADAGF (SEQ ID NO: 3), GRVVQKRSFIEDLLFNKVVTNGLG (SEQ ID NO: 5), and immunogenic fragments thereof; or
(ii) an HIV antigen comprising an amino acid sequence selected from the group consisting of AVGIGAVF (SEQ ID NO: 7), ALGIGAAF (SEQ ID NO: 8), AVGFGAAF (SEQ ID NO: 9), AAGFGAMF (SEQ ID NO: 10), and immunogenic fragments thereof; or
(iii) any combination thereof.

In some embodiments of the composition for use, the genome reduced derivative thereof is characterized by enhanced immunogenicity of the viral antigen relative to immunogenicity of the viral antigen when expressed by a non-genome reduced bacterium upon which the genome reduced derivative thereof is based.

In some embodiments of the composition for use, the genome reduced derivative thereof is a genome reduced derivative of a Gram negative bacterium, optionally an Escherichia coli or a Salmonella bacterium.

In some embodiments of the composition for use, the genome reduced derivative is a genome reduced E. coli.

In some embodiments of the composition for use, the genome reduced derivative is characterized by a reduced number of expressed genes of at least about 1.0%, 2.0%, 3.0%, 4.0%, 5.0%, 6.0%, 7.0%, 8.0%, 9.0%, 10%, 11%, 12%, 13%, 14%, 15%, or greater than 15% of genes relative to a non-genome reduced bacterium upon which the genome reduced bacterium or derivative thereof is based.

In some embodiments of the composition for use, the reduced number of expressed genes comprises a reduction of expressed genes selected from the group consisting of at least about 2.4%, at least about 15.9%, and at least about 29.7% relative to a non-genome reduced bacterium upon which the genome reduced derivative thereof is based.

In some embodiments of the composition for use, the composition for use further comprises a pharmaceutically acceptable carrier, excipient, and/or diluent, optionally wherein the pharmaceutically acceptable carrier, excipient, and/or diluent is pharmaceutically acceptable for use in a human.

In some embodiments, the presently disclosed subject matter provides use of a composition comprising a bacterium or a genome reduced derivative thereof that expresses one or more tandem copies of a polypeptide of interest fused to a bacterial autotransporter β-barrel polypeptide for treating a subject infected with a virus, optionally a coronavirus, further optionally a SARS-CoV virus, a SARS-CoV-2 virus, and/or a porcine epidemic diarrhea virus (PEDV), wherein:
(i) at least one copy of the one or more tandem copies of the polypeptide of interest is modified at or near its N-terminus, to comprise an expression peptide comprising, consisting essentially of, or consisting of one or more copies of the amino acid sequence (X¹X²X³)ₙ, wherein each X¹ is independently selected from the group consisting of Asp (D), Asn (N), Glu (E), Gln (Q), His (H), Arg (R), and Lys (K); each X² is independently selected from the group consisting of Ala (A), Ser(S), Thr (T), Pro (P), Gly (G), Met (M), Leu (L), Ile (I), Val (V), and Cys (C); each X³ is absent in any given member of the one or more copies of the amino acid sequence (X¹X²X³)ₙ or, if present, is independently any amino acid; and n is at least 1; and
(ii) the polypeptide of interest is expressed on a surface of the modified bacterium or the derivative thereof.

Accordingly, it is an object of the presently disclosed subject matter to provide methods and compositions for enhancing the expression of a passenger protein, in one example, on surfaces of bacteria using an expression cassette.

It is a further object of the presently disclosed subject matter to provide methods and compositions for eliciting immune responses to immunogenic viral antigens, such as immunogenic coronavirus (e.g., SARS-CoV, SARS-CoV-2, and/or PEDV) antigens, optionally immunogenic subsequences of the SARS-CoV-2 spike(S) protein such as but not limited to for the purpose of creating new and more effective vaccines and for antibody production.

These and other objects are achieved in whole or in part by the presently disclosed subject matter. Further, representative objects of the presently disclosed subject matter having been stated above, other objects and advantages of the presently disclosed subject matter will become apparent to those skilled in the art after a study of the following Description. Figures, and Sequences.

### BRIEF DESCRIPTION OF THE FIGURES

The presently disclosed subject matter can be better understood by referring to the following FIGURES. The components in the Figures are not necessarily to scale, emphasis instead being placed upon illustrating the principles of the presently disclosed subject matter (often schematically). In the FIGURES, like reference numerals designate corresponding parts throughout the different views. A further understanding of the presently disclosed subject matter can be obtained by reference to an embodiment set forth in the illustrations of the accompanying drawings. Although the illustrated embodiment is merely exemplary of systems for carrying out the presently disclosed subject matter, both the organization and method of operation of the presently disclosed subject matter, in general, together with further objectives and advantages thereof, may be more easily understood by reference to the Figures and the following description. The Figures are not intended to limit the scope of this presently disclosed subject matter, which is set forth with particularity in the claims as appended or as subsequently amended, but merely to clarify and exemplify the presently disclosed subject matter.

For a more complete understanding of the presently disclosed subject matter, reference is now made to the following drawings in which:

FIGS. 1A-1C show enhanced expression of a multimeric passenger protein in an autotransporter by addition of hydrophilic amino acids, including DADA (SEQ ID NO: 9). FIG. 1A is a schematic diagrams of the passenger proteins cloned into the AIDA-I expression cassette of pRAIDA2 (Maeda et al., 2021). FIG. 1B shows an immunoblot (stained with an anti-fusion peptide rabbit polyclonal antibody) in which equal numbers of bacteria were loaded into each lane of the gel. FIG. 1C shows the densitometric quantitation of the bands observed in the immunoblot.

FIG. 2 shows the FP sequences of SARS-Cov-2 (SEQ ID NO: 1) and PEDV (SEQ ID NO: 4) aligned. The core consensus sequence is indicated. These sequences were cloned into expression vector pRAIDA2 (FIG. 2) and transformed into grEc, from which we produced the KWC grEc FP vaccines.

FIGS. 3A and 3B show vaccine platform design and implementation, and schematic diagram of pRAIDA2. FIG. 3A is a map of synthetic plasmid pRAIDA2. Design features include a high copy origin of replication, a kanamycin resistance marker, and an AIDA-I autotransporter expression cassette under the control of a rhamnose-inducible promoter. The expression cassette has a cloning site flanked by BbsI type IIS restriction sites. A trypsin site is engineered into the coding sequence before the beta barrel. FIG. 3B, is a schematic diagram of the general process of candidate vaccine production using pRAIDA2 and grEc.

FIG. 4A-4B shows killed whole cell grEc-Vaccinated Pig Clinical Responses After PEDV Challenge. FIG. 4A shows diarrhea scores following PEDV challenge. FIG. 4B shows body condition scores following PEDV challenge. Both PEDV FP and SARS-CoV-2 FP vaccines provided substantial and highly statistically significant protection against adverse clinical effects observed following the PEDV challenge infection (p<0.01 for all groups. Friedman Rank Sum Test, comparing each vaccinated group to the control for both diarrhea and body condition scores). In these scoring systems, diarrhea scores range from 1 to 3:1, normal to pasty feces: 2, semi-liquid diarrhea with some solid content: 3, liquid diarrhea with no solid content. Body condition scores range from 1 to 3:1, undetectable spinous processes and hook bones: 2, spinous processes and hook bones were slightly felt: 3, spinous processes and hook bones were easily felt and visible.

FIG. 5 shows multiple different SARS-CoV-2 FP vaccines produced for testing. The antigens have different N-terminal modifications, and different amino acid modifications in between the FP segments to enhance export from the bacteria and accessibility in the extracellular medium. As shown in FIG. 5, the “N-terminal motifs” correspond to exemplary expression enhancement peptides of the presently disclosed subject matter, and include the amino acid sequences DA, DADA, DADADA (SEQ ID NO: 105), DADADADA (SEQ ID NO: 106), SASA (SEQ ID NO: 107), SASASASA (SEQ ID NO: 108), KAKA (SEQ ID NO: 109), KAKAKAKA (SEQ ID NO: 110), DAKA (SEQ ID NO: 111), and DAKADAKA (SEQ ID NO: 112), although it is understood that other expression enhancement peptides of the presently disclosed subject matter can also be employed. Additionally. FIG. 5 shows the use of various linkers between the polypeptide of interest (i.e., the “passenger”) and the β-barrel moiety. These linkers include, but are not limited to the amino acid sequences GDG, GDGDG (SEQ ID NO: 101), GSG, GSGSG (SEQ ID NO: 102), GKG, GKGKG (SEQ ID NO: 103), GDGKG (SEQ ID NO: 104), and GGG.

FIG. 6 shows Human anti-FP MAb binding to alternative vaccines by flow cytometry. FIG. 6 also shows the use of various linkers between the polypeptide of interest (i.e., the “passenger”) and the β-barrel moiety. These linkers include, but are not limited to the amino acid sequences GDG, GDGDG (SEQ ID NO: 101), GSG, GSGSG (SEQ ID NO: 102), GKG, GKGKG (SEQ ID NO: 103), GDGKG (SEQ ID NO: 104), and GGG.

FIG. 7 is a table summary of studies testing anti-FP human MAb binding to alternative vaccine constructs by flow cytometry. The vaccine constructs include the use of 5-mer FP antigens, at least one which includes a DADA (2DA: SEQ ID NO: 11) expression enhancement peptides and a linker selected from the group consisting of GDG, GDGDG (SEQ ID NO: 101), GSG, GSGSG (SEQ ID NO: 102), GKG, GKGKG (SEQ ID NO: 103), GDGKG (SEQ ID NO: 104), and GGG.

FIGS. 8A and 8B show the ability of two SARS-CoV-2 FP vaccines to bind human BN anti-FP BN MAb COV 44-79 is stable over 3 freeze-thaw cycles. 5mer 2DA GDG is a vaccine with 5 concatemeric SARS-CoV-2 FP antigens with an N-terminal DADA (SEQ ID NO: 11) motif, wherein the concatemeric SARS-CoV-2 FP antigens are attached to each other with GDG peptide linkers, and 5mer 2DA GDGDG is a vaccine with 5 concatemeric SARS-CoV-2 FP antigens with an N-terminal DADA (SEQ ID NO: 11) motif, wherein the concatemeric SARS-CoV-2 FP antigens are attached to each other with GDGDG (SEQ ID NOs: 104) peptide linkers.

FIG. 9 shows a schematic illustration of the proposed DBTL engineering cycles. In DBTL Cycle 1, we will test the effects on Ag expression and exposure of adding varying numbers of anion (D) amino acid residues to the Ag N-terminal: in Cycle 2, we will test the effects of expressing increasing numbers of FP multimers; in Cycle 3, we will test the effects of adding cationic or neutral hydrophilic amino acid residues to the Ag N-terminal: in Cycle 4 we will test the effects of placing anionic, cationic, or polar neutral charged amino acids in between the individual FP Ags. In Cycle 1, the N-terminal expression enhancement peptides employed are DA, DADA, DADADA, or DADADADA. In Cycle 3, the N-terminal expression enhancement peptides employed are DA. KA. SA, and DAKA. In Cycle 4, the species tested include constructs that have the amino acid sequences AA-FP-GGG-FP-GGG-FP-GGG-FP. AA-FP-GXG-FP-GXG-FP-GXG-FP, or AA-FP-GXGXG-FP-GXGXG-FP-GXGXG-FP-GXGXG-FP-GXGXG-FP, wherein FP represents a fusion peptide sequence: AA represents a polar amino acid between the FP monomers, and X is an amino acid selected from the group consisting of aspartic acid (D), lysine (K), and serine(S).

FIG. 10 shows a schematic illustration of Immunogenicity comparison vaccination schema.

FIG. 11 shows a table of Vaccinations: Antigens and Doses. The Prototype FP is the original monomeric FP. Mod 1 FP. Mod 2 FP, and Mod 3 FP are the three selected modified/multimeric FP constructs as judged by anti-FP binding in flow cytometry experiments.

### BRIEF DESCRIPTION OF THE SEQUENCE LISTING

SEQ ID NOs: 1-4 are the amino acid sequences of exemplary SARS-CoV-2 fusion peptides that can be employed as monomers or multimers in the compositions and methods of the presently disclosed subject matter.

SEQ ID NO: 5 is the amino acid sequence of an exemplary PEDV fusion peptide that can be employed as a monomer or a multimer in the compositions and methods of the presently disclosed subject matter.

SEQ ID NO: 6 is a consensus sequence of the exemplary fusion peptide sequences of SEQ ID NOs: 2 and 5.

SEQ ID NOs: 7-10 are the amino acid sequences of exemplary HIV antigenic peptides that can be employed as monomers or multimers in the compositions and methods of the presently disclosed subject matter.

SEQ ID NOs: 11-86 are the amino acid sequences of exemplary tetrapeptides that can be employed as monomers, multimers, and/or as part of the expression enhancement peptides of the presently disclosed subject matter.

SEQ ID NOs: 87-90 and 101-103 are the amino acid sequences of exemplary peptide linkers that can be employed as monomers or multimers in the compositions and methods of the presently disclosed subject matter.

SEQ ID NO: 91 is the amino acid sequence of an HA tag that can be employed in the compositions and methods of the presently disclosed subject matter.

SEQ ID NO: 92 is the nucleotide sequence of an exemplary autotransporter (AT) expression vector of the presently disclosed subject matter comprising a monomer of a SARS-CoV-2 fusion peptide.

SEQ ID NO: 93 is the nucleotide sequence of an exemplary autotransporter (AT) expression vector of the presently disclosed subject matter comprising a trimer of a SARS-CoV-2 fusion peptide.

SEQ ID NO: 94 is the nucleotide sequence of an exemplary autotransporter (AT) expression vector of the presently disclosed subject matter comprising a pentamer of a SARS-CoV-2 fusion peptide.

SEQ ID NO: 95 is the amino acid sequence of an exemplary SARS-CoV-2 fusion peptide monomer with a C-terminal linker.

SEQ ID NO: 96 is the amino acid sequence of an exemplary SARS-CoV-2 fusion peptide trimer, with the members of the trimer linked via a peptide linker.

SEQ ID NO: 97 is the amino acid sequence of an exemplary SARS-CoV-2 fusion peptide pentamer, with the members of the pentamer linked via a peptide linker.

SEQ ID NO: 98 is the nucleotide sequence of an exemplary autotransporter (AT) expression vector of the presently disclosed subject matter comprising a pentamer of a SARS-CoV-2 fusion peptide with the exemplary expression enhancement peptide DADA (SEQ ID NO: 9) at the N-terminus of the first member of the pentamer.

SEQ ID NO: 99 is the amino acid sequence of the SARS-CoV-2 fusion peptide pentamer with the exemplary expression enhancement peptide DADA (SEQ ID NO: 9) at its N-terminus.

SEQ ID NO: 100 is the amino acid sequence of an exemplary autotransporter beta-barrel polypeptide that can be employed in the compositions and methods of the presently disclosed subject matter. It corresponds to amino acids 962-1286 of Accession No. Q03155.1 of the GENBANK® biosequence database.

SEQ ID NOs: 104-111 are the amino acid sequences of additional exemplary peptides that can be employed as monomers, multimers, and/or as part of the expression enhancement peptides of the presently disclosed subject matter.

### DETAILED DESCRIPTION

The presently disclosed subject matter now will be described more fully hereinafter, in which some, but not all embodiments of the presently disclosed subject matter are described. Indeed, the presently disclosed subject matter can be embodied in many different forms and should not be construed as limited to the embodiments set forth herein: rather, these embodiments are provided so that this disclosure will satisfy applicable legal requirements.

### I. Definitions

While the following terms are believed to be well understood by one of ordinary skill in the art, the following definitions are set forth to facilitate explanation of the presently disclosed subject matter.

Unless defined otherwise, all technical and scientific terms used herein have the same meaning as commonly understood to one of ordinary skill in the art to which the presently disclosed subject matter belongs.

Following long-standing patent law convention, the terms “a”. “an”, and “the” refer to “one or more” when used in this application, including the claims.

The term “and/or” when used in describing two or more items or conditions, refers to situations where all named items or conditions are present or applicable, or to situations wherein only one (or less than all) of the items or conditions is present or applicable.

The use of the term “or” in the claims is used to mean “and/or” unless explicitly indicated to refer to alternatives only or the alternatives are mutually exclusive, although the disclosure supports a definition that refers to only alternatives and “and/or”. As used herein “another” can mean at least a second or more.

The term “comprising”, which is synonymous with “including”. “containing”, or “characterized by” is inclusive or open-ended and does not exclude additional, unrecited elements or method steps. “Comprising” is a term of art used in claim language which means that the named elements are essential, but other elements can be added and still form a construct within the scope of the claim.

As used herein, the phrase “consisting of” excludes any element, step, or ingredient not specified in the claim. When the phrase “consists of” appears in a clause of the body of a claim, rather than immediately following the preamble, it limits only the element set forth in that clause; other elements are not excluded from the claim as a whole.

As used herein, the phrase “consisting essentially of” limits the scope of a claim to the specified materials or steps, plus those that do not materially affect the basic and novel characteristic(s) of the claimed subject matter.

With respect to the terms “comprising”. “consisting of”, and “consisting essentially of”, where one of these three terms is used herein, the presently disclosed and claimed subject matter can include the use of either of the other two terms.

Unless otherwise indicated, all numbers expressing quantities of time, temperature, weight, concentration, volume, strength, speed, length, width, and so forth used in the specification and claims are to be understood as being modified in all instances by the term “about”. Accordingly, unless indicated to the contrary, the numerical parameters set forth in this specification and attached claims are approximations that can vary depending upon the desired properties sought to be obtained by the presently disclosed subject matter.

As used herein, the term “about”, when referring to a value is meant to encompass variations of in one example ±20% or ±10%, in another example ±5%, in another example ±1%, and in still another example ±0.1% from the specified amount, as such variations are appropriate to perform the disclosed methods.

Numerical ranges recited herein by endpoints include all numbers and fractions subsumed within that range (e.g., 1 to 5 includes 1, 1.5, 2, 2.75, 3, 3.90, 4, 4.24, and 5). Similarly, numerical ranges recited herein by endpoints include subranges subsumed within that range (e.g., 1 to 5 includes 1-1.5, 1.5-2, 2-2.75, 2.75-3, 3-3.90, 3.90-4, 4-4.24, 4.24-5, 2-5, 3-5, 1-4, and 2-4).

A disease or disorder is “alleviated” if the severity of a symptom of the disease, condition, or disorder, or the frequency with which such a symptom is experienced by a subject, or both, are reduced.

The terms “additional therapeutically active compound” or “additional therapeutic agent”, as used in the context of the presently disclosed subject matter, refers to the use or administration of a compound for an additional therapeutic use for a particular injury, disease, or disorder being treated. Such a compound, for example, could include one being used to treat an unrelated disease or disorder, or a disease or disorder which may not be responsive to the primary treatment for the injury, disease or disorder being treated.

As used herein, the term “adjuvant” refers to a substance that elicits an enhanced immune response when used in combination with a specific antigen. In some embodiments, the bacteria expressing antigens as described herein are combined with different adjuvants.

As use herein, the terms “administration of” and or “administering” a compound should be understood to mean providing a compound of the presently disclosed subject matter or a prodrug of a compound of the presently disclosed subject matter to a subject in need of treatment.

As used herein, the term “aerosol” refers to suspension in the air. In particular, aerosol refers to the particularization or atomization of a formulation of the presently disclosed subject matter and its suspension in the air.

As used herein, an “analog” of a chemical compound is a compound that, by way of example, resembles another in structure but is not necessarily an isomer (e.g., 5-fluorouracil is an analog of thymine).

As used herein. “amino acids” are represented by the full name thereof, by the three letter code corresponding thereto, or by the one-letter code corresponding thereto, as indicated in Table 1:

TABLE 1
Table of the Genetic Code

| | 3-Letter | 1-Letter | |
|---|---|---|---|
| Full Name | Code | Code | Functionally Equivalent Codons |
| Aspartic Acid | Asp | D | GAC GAU |
| Glutamic Acid | Glu | E | GAA GAG |
| Lysine | Lys | K | AAA AAG |
| Arginine | Arg | R | AGA AGG CGA CGC CGG CGU |
| Histidine | His | H | CAC CAU |
| Tyrosine | Tyr | Y | UAC UAU |
| Cysteine | Cys | C | UGC UGU |
| Asparagine | Asn | N | AAC AAU |
| Glutamine | Gln | Q | CAA CAG |
| Serine | Ser | S | ACG AGU UCA UCC UCG UCU |
| Threonine | Thr | T | ACA ACC ACG ACU |
| Glycine | Gly | G | GGA GGC GGG GGU |
| Alanine | Ala | A | GCA GCC GCG GCU |
| Valine | Val | V | GUA GUC GUG GUU |
| Leucine | Leu | L | UUA UUG CUA CUC CUG CUU |
| Isoleucine | Ile | I | AUA AUC AUU |
| Methionine | Met | M | AUG |
| Proline | Pro | P | CCA CCC CCG CCU |
| Phenylalanine | Phe | F | UUC UUU |
| Tryptophan | Trp | W | UGG |

The term “amino acid” is used interchangeably with “amino acid residue”, and may refer to a free amino acid and to an amino acid residue of a peptide. It will be apparent from the context in which the term is used whether it refers to a free amino acid or a residue of a peptide.

The expression “amino acid” as used herein is meant to include both natural and synthetic amino acids, and both D and L amino acids. “Standard amino acid” means any of the twenty standard L-amino acids commonly found in naturally occurring peptides. “Nonstandard amino acid residue” means any amino acid, other than the standard amino acids, regardless of whether it is prepared synthetically or derived from a natural source. As used herein, “synthetic amino acid” also encompasses chemically modified amino acids, including but not limited to salts, amino acid derivatives (such as amides), and substitutions. Amino acids contained within the peptides of the presently disclosed subject matter, and particularly at the carboxy- or amino-terminus, can be modified by methylation, amidation, acetylation or substitution with other chemical groups which can change the peptide's circulating half-life without adversely affecting their activity. Additionally, a disulfide linkage may be present or absent in the peptides of the presently disclosed subject matter.

Amino acids may be classified into seven groups on the basis of the side chain R: (1) aliphatic side chains; (2) side chains containing a hydroxylic (OH) group; (3) side chains containing sulfur atoms; (4) side chains containing an acidic or amide group; (5) side chains containing a basic group; (6) side chains containing an aromatic ring; and (7) proline, an amino acid in which the side chain is fused to the amino group.

Synthetic or non-naturally occurring amino acids refer to amino acids which do not naturally occur in vivo but which, nevertheless, can be incorporated into the peptide structures described herein. The resulting “synthetic peptide” contain amino acids other than the 20 naturally occurring, genetically encoded amino acids at one, two, or more positions of the peptides. For instance, naphthylalanine can be substituted for tryptophan to facilitate synthesis. Other synthetic amino acids that can be substituted into peptides include L-hydroxypropyl, L-3,4-dihydrooxyphenylalanyl, α-amino acids such as L-α-hydroxylysyl and D-α-methylalanyl, L-α-methylalanyl, β-amino acids, and isoquinolyl. D amino acids and non-naturally occurring synthetic amino acids can also be incorporated into the peptides. Other derivatives include replacement of the naturally occurring side chains of the 20 genetically encoded amino acids (or any L or D amino acid) with other side chains.

In some embodiments, the compositions and methods of the presently disclosed subject matter employ an expression enhancer amino acid or an expression enhancer peptide, which in some embodiments is a dipeptide, tripeptide, or a concatemer of one or more dipeptides or tripeptides. An exemplary expression enhancer peptide for use in the compositions and methods of the presently disclosed subject matter is the dipeptide Asp-Ala (DA), which is some embodiments is dimerized to the tetrapeptide Asp-Ala-Asp-Ala (DADA). It is noted, however, that the dipeptide DA and the tetrapeptide DADA are merely exemplary, and that conservative amino acid substitutions of the Asp or the Ala are also employable within the scope of the presently disclosed subject matter. As used herein, the term “conservative amino acid substitution” is defined herein as exchanges within one of the following five groups:
I. Small aliphatic, nonpolar or slightly polar residues: Ala, Ser, Thr, Pro, Gly;
II. Polar, negatively charged residues and their amides: Asp, Asn, Glu, Gln;
III. Polar, positively charged residues: His, Arg, Lys;
IV. Large, aliphatic, nonpolar residues: Met, Leu, Ile, Val, Cys
V. Large, aromatic residues: Phe, Tyr, Trp

Thus, with respect to expression enhancer peptide for use in the compositions and methods of the presently disclosed subject matter, the expression enhancer peptide can be in some embodiments DA or conservatively substituted versions thereof, and in some embodiments concatemers of DA or conservative substituted versions thereof. By way of example and not limitation, the expression enhancer peptides for use in the compositions and methods of the presently disclosed subject matter can be described as monomers or multimers (e.g., dimers) of the amino acid sequence (X₁X₂)ₙ, wherein X₁ is selected from the group consisting of Asp, Asn, Glu, Gln, His, Arg, and Lys; X₂ is selected from the group consisting of Ala, Ser, Thr, Pro, Gly, Met, Leu, Ile, Val, and Cys and n is 1, 2, 3, 4, 5, 6, 7, 8, 9, 10, or more. It is further noted that in those embodiments wherein X₁X₂ is multimerized (i.e., n>1, including but not limited to n=2), each individual X₁ and X₂ can be different. By way of example and not limitation, therefore, the expression enhancer peptides for use in the compositions and methods of the presently disclosed subject matter in some embodiments can include X₁=D, N, E, H, R, K or Q, and in some embodiments can include X₂=A, S, T, P, G, M, L, I, V, or C. As such, the expression enhancer peptides for use in the compositions and methods of the presently disclosed subject matter can be in some embodiments X₁ₐX₂ₐX_{1b}X_{2b}, Wherein X₁ₐ and X_{1b} are the same or a different amino acid and X₂ₐ and X_{2b} are the same or a different amino acid. Thus, particular non-limiting examples of the expression enhancer peptides for use in the compositions and methods of the presently disclosed subject matter include DA, DS, DT, DP, DG, NA, NS, NT, NP, NG, EA, ES, ET, EP, QA, QG, QA, QS, QT, QP, QG, HA, HS, HT, HP, HG, RA, RS, RT, RP, RG, LA, LS, LT, LP, LG, and any combination thereof including but not limited to DADA (SEQ ID NO: 11), EAEA (SEQ ID NO: 12), DAEA (SEQ ID NO: 13), EADA (SEQ ID NO: 14), NANA (SEQ ID NO: 15), QAQA (SEQ ID NO: 16), DANA (SEQ ID NO: 17), EANA (SEQ ID NO: 18), NADA (SEQ ID NO: 19), NAQA (SEQ ID NO: 20), HAHA (SEQ ID NO: 21), RARA (SEQ ID NO: 22), LALA (SEQ ID NO: 23), HSHS (SEQ ID NO: 24), HTHT (SEQ ID NO: 25), LSLS (SEQ ID NO: 26), LTLT (SEQ ID NO: 27), RSRS (SEQ ID NO: 28), RTRT (SEQ ID NO: 29), HPHP (SEQ ID NO: 30), HGHG (SEQ ID NO: 31), LPLP (SEQ ID NO: 32), LGLG (SEQ ID NO: 33), DSDS (SEQ ID NO: 34), ESES (SEQ ID NO: 35), DSES (SEQ ID NO: 36), ESDS (SEQ ID NO: 37), NSNS (SEQ ID NO: 38), QSQS (SEQ ID NO: 39), DSNS (SEQ ID NO: 40), ESNS (SEQ ID NO: 41), NSDS (SEQ ID NO: 42), NSQS (SEQ ID NO: 43), DTDT (SEQ ID NO: 44), ETET (SEQ ID NO: 45), DTET (SEQ ID NO: 46), ETDT (SEQ ID NO: 47), NTNT (SEQ ID NO: 48), QTQT (SEQ ID NO: 49), DTNT (SEQ ID NO: 50), ETNT (SEQ ID NO: 51), NTDT (SEQ ID NO: 52), NTQT (SEQ ID NO: 53), DPDP (SEQ ID NO: 54), EPEP (SEQ ID NO: 55), DPEP (SEQ ID NO: 56), EPDP (SEQ ID NO: 57), NPNP (SEQ ID NO: 58), QPQP (SEQ ID NO: 59), DPNP (SEQ ID NO: 60), EPNP (SEQ ID NO: 61). NPDP (SEQ ID NO: 62), NPQP (SEQ ID NO: 63), DGDG (SEQ ID NO: 64), EGEG (SEQ ID NO: 65), DGEG (SEQ ID NO: 66), EGDG (SEQ ID NO: 67), NGNG (SEQ ID NO: 68), QGQG (SEQ ID NO: 69), DGNG (SEQ ID NO: 70), EGNG (SEQ ID NO: 71), NGDG (SEQ ID NO: 72), NGQG (SEQ ID NO: 73), HAHS (SEQ ID NO: 74), RARS (SEQ ID NO: 75), LALS (SEQ ID NO: 76). HSHT (SEQ ID NO: 77), HTHP (SEQ ID NO: 78), LSLT (SEQ ID NO: 79), LTLP (SEQ ID NO: 80), RSRT (SEQ ID NO: 81), RTRP (SEQ ID NO: 82), HPHG (SEQ ID NO: 83), HGHA (SEQ ID NO: 84), LPLA (SEQ ID NO: 85), LGLA (SEQ ID NO: 86), etc.

The nomenclature used to describe the peptide compounds of the presently disclosed subject matter follows the conventional practice wherein the amino group is presented to the left and the carboxy group to the right of each amino acid residue. In the formulae representing selected specific embodiments of the presently disclosed subject matter, the amino- and carboxy-terminal groups, although not specifically shown, will be understood to be in the form they would assume at physiologic pH values, unless otherwise specified.

The term “basic” or “positively charged” amino acid, as used herein, refers to amino acids in which the R groups have a net positive charge at pH 7.0, and include, but are not limited to, the standard amino acids lysine, arginine, and histidine.

As used herein, an “analog” of a chemical compound is a compound that, by way of example, resembles another in structure but is not necessarily an isomer (e.g., 5-fluorouracil is an analog of thymine).

The term “antibody”, as used herein, refers to an immunoglobulin molecule which is able to specifically bind to a specific epitope on an antigen. Antibodies can be intact immunoglobulins derived from natural sources or from recombinant sources and can be immunoreactive portions of intact immunoglobulins. Antibodies are typically tetramers of immunoglobulin molecules. The antibodies in the presently disclosed subject matter may exist in a variety of forms including, for example, polyclonal antibodies, monoclonal antibodies, Fv, Fab and F(ab)₂, as well as single chain antibodies and humanized antibodies.

An “antibody heavy chain”, as used herein, refers to the larger of the two types of polypeptide chains present in all antibody molecules.

An “antibody light chain”, as used herein, refers to the smaller of the two types of polypeptide chains present in all antibody molecules.

By the term “synthetic antibody” as used herein, is meant an antibody which is generated using recombinant DNA technology, such as, for example, an antibody expressed by a bacteriophage as described herein. The term should also be construed to mean an antibody which has been generated by the synthesis of a DNA molecule encoding the antibody and which DNA molecule expresses an antibody protein, or an amino acid sequence specifying the antibody, wherein the DNA or amino acid sequence has been obtained using synthetic DNA or amino acid sequence technology which is available and well known in the art.

The term “antigen” as used herein is defined as a molecule that provokes an immune response. This immune response may involve either antibody production, or the activation of specific immunologically competent cells, or both. An antigen can be derived from organisms, subunits of proteins/antigens, killed or inactivated whole cells or lysates. The term “immunogen” is used interchangeably with “antigen” herein. The term antigen can be designed to elicit an immune response, An antigen can come from a pathogen. An antigen can come from a virus. An antigen can come from a biologically active molecule. An antigen can come from a molecule involved in an autoimmune process. An antigen may elicit an immune response to develop humor and/or cellular immunity. An antigen may elicit an immune response to develop humor and/or cellular immune responses to develop custom antibodies and/or custom cell mediated immune responses. An antigen may come from a cancer cell. An antigen may be selected form the group consisting of SARS-CoV-2, HIV, PEDV, HIV and any combination thereof. In some embodiments, an antigen may be the binding site of a neutralizing monoclonal or polyclonal antibody, or antibody that is toxic for cancer cells, or affects a tissue, cell, or organ in a desired way. In some embodiments, an antigen is capable of eliciting a broadly neutralizing immune response, for example because it shows low or minimal diversity or evolution over many sequenced strains.

The antigen can be any desired antigen as would be apparent to one of ordinary skill in the art upon a review of the instant disclosure. In some embodiments, the antigen is derived from a microbe. In some embodiments, the antigen is derived from a cancer. In some embodiments the antigen is derived from a host protein that mediates other diseases or undesirable phenotypes, including in some embodiments autoimmune or inflammatory diseases, or diseases in which the expression of a particular host protein mediates a disease process. In some embodiments, the antigen is derived from a cancer, or a target of an inappropriate or undesirable immune response, or a component of the host immune system, such as (but not exclusively), a host immune system component that, when targeted for destruction or inactivation or activation, alter an undesirable immune response.

In some embodiments, the recombinant protein is an antigen, optionally an antigen on a surface of a membrane, or a derivative thereof. The antigen or immunogen is any antigen against which an immune response is desired. Representative, non-limiting examples of antigens include an antigen to modulate autoimmune responses, an antigen for which it might be therapeutically useful to produce an immune response, such as fibrosis associated with atherosclerosis or the amyloid plaques of Alzheimer's disease or other degenerative diseases: an antigen used to induce an immune response against specific components of the immune system to modify autoimmune or allergic diseases; and/or combinations thereof. In some embodiments, the antigen is derived from cytokine and/or chemokine targets, immune checkpoint molecules (e.g., PD-1 etc., targeted by therapeutic monoclonal antibodies), proteins involved in the clotting cascade, in blood pressure regulation, inflammatory factors (TNF, ILs).

The term “antigenic determinant” as used herein refers to that portion of an antigen that makes contact with a particular antibody (i.e., an epitope). When a protein or fragment of a protein, or chemical moiety is used to immunize a host animal, numerous regions of the antigen may induce the production of antibodies that bind specifically to a given region or three-dimensional structure on the protein: these regions or structures are referred to as antigenic determinants. An antigenic determinant may compete with the intact antigen (i.e., the “immunogen” used to elicit the immune response) for binding to an antibody.

The term “antimicrobial agents” as used herein refers to any naturally-occurring, synthetic, or semi-synthetic compound or composition or mixture thereof, which is safe for human or animal use as practiced in the methods of this presently disclosed subject matter, and is effective in killing or substantially inhibiting the growth of microbes. “Antimicrobial” as used herein, includes antibacterial, antifungal, and antiviral agents.

The term “aqueous solution” as used herein can include other ingredients commonly used, such as sodium bicarbonate described herein, and further includes any acid or base solution used to adjust the pH of the aqueous solution while solubilizing a peptide.

The term “binding” refers to the adherence of molecules to one another, such as, but not limited to, enzymes to substrates, ligands to receptors, antibodies to antigens. DNA binding domains of proteins to DNA, and DNA or RNA strands to complementary strands.

“Binding partner”, as used herein, refers to a molecule capable of binding to another molecule.

The term “biocompatible”, as used herein, refers to a material that does not elicit a substantial detrimental response in the host.

As used herein, the term “biologically active fragments” or “bioactive fragment” of the peptides encompasses natural or synthetic portions of a longer peptide or protein that are capable of specific binding to their natural ligand or of performing the desired function of the protein, for example, a fragment of a protein of larger peptide which still contains the epitope of interest and is immunogenic.

The term “biological sample”, as used herein, refers to samples obtained from a subject, including, but not limited to, skin, hair, tissue, blood, plasma, cells, sweat and urine.

As used herein, the term “carrier molecule” refers to any molecule that is chemically conjugated to the antigen of interest that enables an immune response resulting in antibodies specific to the native antigen.

As used herein, the term “chemically conjugated”, or “conjugating chemically” refers to linking the antigen to the carrier molecule. This linking can occur on the genetic level using recombinant technology, wherein a hybrid protein may be produced containing the amino acid sequences, or portions thereof, of both the antigen and the carrier molecule. This hybrid protein is produced by an oligonucleotide sequence encoding both the antigen and the carrier molecule, or portions thereof. This linking also includes covalent bonds created between the antigen and the carrier protein using other chemical reactions, such as, but not limited to glutaraldehyde reactions. Covalent bonds may also be created using a third molecule bridging the antigen to the carrier molecule. These cross-linkers are able to react with groups, such as but not limited to, primary amines, sulfhydryls, carbonyls, carbohydrates, or carboxylic acids, on the antigen and the carrier molecule. Chemical conjugation also includes non-covalent linkage between the antigen and the carrier molecule.

A “coding region” of a gene comprises the nucleotide residues of the coding strand of the gene and the nucleotides of the non-coding strand of the gene which are homologous with or complementary to, respectively, the coding region of an mRNA molecule which is produced by transcription of the gene.

The term “competitive sequence” refers to a peptide or a modification, fragment, derivative, or homolog thereof that competes with another peptide for its cognate binding site.

“Complementary” as used herein refers to the broad concept of subunit sequence complementarity between two nucleic acids, e.g., two DNA molecules. When a nucleotide position in both of the molecules is occupied by nucleotides normally capable of base pairing with each other, then the nucleic acids are considered to be complementary to each other at this position. Thus, two nucleic acids are complementary to each other when a substantial number (at least 50%) of corresponding positions in each of the molecules are occupied by nucleotides which normally base pair with each other (e.g., A:T and G:C nucleotide pairs). Thus, it is known that an adenine residue of a first nucleic acid region is capable of forming specific hydrogen bonds (“base pairing”) with a residue of a second nucleic acid region which is antiparallel to the first region if the residue is thymine or uracil. Similarly, it is known that a cytosine residue of a first nucleic acid strand is capable of base pairing with a residue of a second nucleic acid strand which is antiparallel to the first strand if the residue is guanine. A first region of a nucleic acid is complementary to a second region of the same or a different nucleic acid if, when the two regions are arranged in an antiparallel fashion, at least one nucleotide residue of the first region is capable of base pairing with a residue of the second region. Preferably, the first region comprises a first portion and the second region comprises a second portion, whereby, when the first and second portions are arranged in an antiparallel fashion, at least about 50%, and preferably at least about 75%, at least about 90%, or at least about 95% of the nucleotide residues of the first portion are capable of base pairing with nucleotide residues in the second portion. More preferably, all nucleotide residues of the first portion are capable of base pairing with nucleotide residues in the second portion.

A “compound”, as used herein, refers to a polypeptide, an isolated nucleic acid, or other agent used in the method of the presently disclosed subject matter.

A “control” cell, tissue, sample, or subject is a cell, tissue, sample, or subject of the same type as a test cell, tissue, sample, or subject. The control may, for example, be examined at precisely or nearly the same time the test cell, tissue, sample, or subject is examined. The control may also, for example, be examined at a time distant from the time at which the test cell, tissue, sample, or subject is examined, and the results of the examination of the control may be recorded so that the recorded results may be compared with results obtained by examination of a test cell, tissue, sample, or subject. The control may also be obtained from another source or similar source other than the test group or a test subject, where the test sample is obtained from a subject suspected of having a disease or disorder for which the test is being performed.

A “test” cell is a cell being examined.

A “pathoindicative” cell is a cell which, when present in a tissue, is an indication that the animal in which the tissue is located (or from which the tissue was obtained) is afflicted with a disease or disorder.

A “pathogenic” cell is a cell which, when present in a tissue, causes or contributes to a disease or disorder in the animal in which the tissue is located (or from which the tissue was obtained).

A tissue “normally comprises” a cell if one or more of the cell are present in the tissue in an animal not afflicted with a disease or disorder.

As used herein, a “derivative” of a bacterium, antigen, composition or other compound refers to a bacterium, antigen, composition or other compound that may be produced from bacterium, antigen, composition or other compound of similar structure in one or more steps.

The use of the word “detect” and its grammatical variants refers to measurement of the species without quantification, whereas use of the word “determine” or “measure” with their grammatical variants are meant to refer to measurement of the species with quantification. The terms “detect” and “identify” are used interchangeably herein.

As used herein, a “detectable marker” or a “reporter molecule” is an atom or a molecule that permits the specific detection of a compound comprising the marker in the presence of similar compounds without a marker. Detectable markers or reporter molecules include, e.g., radioactive isotopes, antigenic determinants, enzymes, nucleic acids available for hybridization, chromophores, fluorophores, chemiluminescent molecules, electrochemically detectable molecules, and molecules that provide for altered fluorescence-polarization or altered light-scattering.

As used herein, the term “diagnosis” refers to detecting a risk or propensity to an addictive related disease disorder. In any method of diagnosis exist false positives and false negatives. Any one method of diagnosis does not provide 100% accuracy.

A “disease” is a state of health of an animal wherein the animal cannot maintain homeostasis, and wherein if the disease is not ameliorated then the animal's health continues to deteriorate.

In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

As used herein, the term “domain” refers to a part of a molecule or structure that shares common physicochemical features, such as, but not limited to, hydrophobic, polar, globular and helical domains or properties such as ligand binding, signal transduction, cell penetration and the like. Specific examples of binding domains include, but are not limited to DNA binding domains and ATP binding domains.

As used herein, an “effective amount” or “therapeutically effective amount” means an amount sufficient to produce a selected effect, such as alleviating symptoms of a disease or disorder. In the context of administering compounds in the form of a combination, such as multiple compounds, the amount of each compound, when administered in combination with another compound(s), may be different from when that compound is administered alone. Thus, an effective amount of a combination of compounds refers collectively to the combination as a whole, although the actual amounts of each compound may vary. The term “more effective” means that the selected effect is alleviated to a greater extent by one treatment relative to the second treatment to which it is being compared.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

The term “epitope” as used herein is defined as small chemical groups on the antigen molecule that can elicit and react with an antibody. An antigen can have one or more epitopes. Most antigens have many epitopes: i.e., they are multivalent. In general, an epitope is roughly at least five amino acids or sugars in size. One skilled in the art understands that generally the overall three-dimensional structure, rather than the specific linear sequence of the molecule, is the main criterion of antigenic specificity.

As used herein, an “essentially pure” preparation of a particular protein or peptide is a preparation wherein at least about 95%, and preferably at least about 99%, by weight, of the protein or peptide in the preparation is the particular protein or peptide.

A “fragment” or “segment” is a portion of an amino acid sequence, comprising at least one amino acid, or a portion of a nucleic acid sequence comprising at least one nucleotide. The terms “fragment” and “segment” are used interchangeably herein.

As used herein, the term “fragment”, as applied to a protein or peptide, can ordinarily be at least about 3-15 amino acids in length, at least about 15-25 amino acids, at least about 25-50 amino acids in length, at least about 50-75 amino acids in length, at least about 75-100 amino acids in length, and greater than 100 amino acids in length.

As used herein, the term “fragment” as applied to a nucleic acid, may ordinarily be at least about 20 nucleotides in length, typically, at least about 50 nucleotides, more typically, from about 50 to about 100 nucleotides, preferably, at least about 100 to about 200 nucleotides, even more preferably, at least about 200 nucleotides to about 300 nucleotides, yet even more preferably, at least about 300 to about 350, even more preferably, at least about 350 nucleotides to about 500 nucleotides, yet even more preferably, at least about 500 to about 600, even more preferably, at least about 600 nucleotides to about 620 nucleotides, yet even more preferably, at least about 620 to about 650, and most preferably, the nucleic acid fragment will be greater than about 650 nucleotides in length.

The terms “fragment” and “segment” are used interchangeably herein.

As used herein, a “functional” biological molecule is a biological molecule in a form in which it exhibits a property by which it is characterized. A functional enzyme, for example, is one which exhibits the characteristic catalytic activity by which the enzyme is characterized.

“Homologous” as used herein, refers to the subunit sequence similarity between two polymeric molecules, e.g., between two nucleic acid molecules, e.g., two DNA molecules or two RNA molecules, or between two polypeptide molecules. When a subunit position in both of the two molecules is occupied by the same monomeric subunit, e.g., if a position in each of two DNA molecules is occupied by adenine, then they are homologous at that position. The homology between two sequences is a direct function of the number of matching or homologous positions, e.g., if half (e.g., five positions in a polymer ten subunits in length) of the positions in two compound sequences are homologous then the two sequences are 50% homologous, if 90% of the positions, e.g., 9 of 10, are matched or homologous, the two sequences share 90% homology. By way of example, the DNA sequences 5′-ATTGCC-3′ and 5′-TATGGC-3′ share 50% homology.

As used herein. “homology” is used synonymously with “identity”.

The determination of percent identity between two nucleotide or amino acid sequences can be accomplished using a mathematical algorithm. For example, a mathematical algorithm useful for comparing two sequences is the algorithm of Karlin & Altschul, 1990, modified as in Karlin & Altschul, 1993. This algorithm is incorporated into the NBLAST and XBLAST programs of Altschul et al., 1990a, and can be accessed, for example at the National Center for Biotechnology Information (NCBI) world wide web site. BLAST nucleotide searches can be performed with the NBLAST program (designated “blastn” at the NCBI web site), using the following parameters: gap penalty=5; gap extension penalty=2; mismatch penalty=3; match reward=1; expectation value 10.0; and word size=11 to obtain nucleotide sequences homologous to a nucleic acid described herein. BLAST protein searches can be performed with the XBLAST program (designated “blastn” at the NCBI web site) or the NCBI “blastp” program, using the following parameters: expectation value 10.0, BLOSUM62 scoring matrix to obtain amino acid sequences homologous to a protein molecule described herein. To obtain gapped alignments for comparison purposes. Gapped BLAST can be utilized as described in Altschul et al., 1997. Alternatively. PSI-Blast or PHI-Blast can be used to perform an iterated search which detects distant relationships between molecules (Altschul et al., 1997) and relationships between molecules which share a common pattern. When utilizing BLAST. Gapped BLAST. PSI-Blast, and PHI-Blast programs, the default parameters of the respective programs (e.g., XBLAST and NBLAST) can be used.

The percent identity between two sequences can be determined using techniques similar to those described above, with or without allowing gaps. In calculating percent identity, typically exact matches are counted

As used herein, the term “hybridization” is used in reference to the pairing of complementary nucleic acids. Hybridization and the strength of hybridization (i.e., the strength of the association between the nucleic acids) is impacted by such factors as the degree of complementarity between the nucleic acids, stringency of the conditions involved, the length of the formed hybrid, and the G:C ratio within the nucleic acids.

By the term “immunizing a subject against an antigen” is meant administering to the subject a composition, a protein complex, a DNA encoding a protein complex, an antibody or a DNA encoding an antibody, which elicits an immune response in the subject, and, for example, provides protection to the subject against a disease caused by the antigen or which prevents the function of the antigen.

The term “immunologically active fragments thereof” will generally be understood in the art to refer to a fragment of a polypeptide antigen comprising at least an epitope, which means that the fragment at least comprises 4 contiguous amino acids from the sequence of the polypeptide antigen.

As used herein, the term “inhaler” refers both to devices for nasal and pulmonary administration of a drug, e.g., in solution, powder and the like. For example, the term “inhaler” is intended to encompass a propellant driven inhaler, such as is used to administer antihistamine for acute asthma attacks, and plastic spray bottles, such as are used to administer decongestants.

The term “inhibit”, as used herein when referring to a function, refers to the ability of a compound of the presently disclosed subject matter to reduce or impede a described function. In some embodiments, inhibition is by at least 10%, in some embodiments by at least 25%, in some embodiments by at least 50%, and in some embodiments, the function is inhibited by at least 75%. When the term “inhibit” is used more generally, such as “inhibit Factor I”, it refers to inhibiting expression, levels, and activity of Factor I.

The term “inhibit a complex”, as used herein, refers to inhibiting the formation of a complex or interaction of two or more proteins, as well as inhibiting the function or activity of the complex. The term also encompasses disrupting a formed complex. However, the term does not imply that each and every one of these functions must be inhibited at the same time.

The term “inhibit a protein”, as used herein, refers to any method or technique which inhibits protein synthesis, levels, activity, or function, as well as methods of inhibiting the induction or stimulation of synthesis, levels, activity, or function of the protein of interest. The term also refers to any metabolic or regulatory pathway which can regulate the synthesis, levels, activity, or function of the protein of interest. The term includes binding with other molecules and complex formation. Therefore, the term “protein inhibitor” refers to any agent or compound, the application of which results in the inhibition of protein function or protein pathway function. However, the term does not imply that each and every one of these functions must be inhibited at the same time.

As used herein “injecting, or applying, or administering” includes administration of a compound of the presently disclosed subject matter by any number of routes and means including, but not limited to, topical, oral, buccal, intravenous, intramuscular, intra-arterial, intramedullary, intrathecal, intraventricular, transdermal, subcutaneous, intraperitoneal, intranasal, enteral, topical, sublingual, vaginal, ophthalmic, pulmonary, vaginal, or rectal approaches.

As used herein, an “instructional material” includes a publication, a recording, a diagram, or any other medium of expression which can be used to communicate the usefulness of the peptide of the presently disclosed subject matter in the kit for effecting alleviation of the various diseases or disorders recited herein. Optionally, or alternately, the instructional material may describe one or more methods of alleviating the diseases or disorders in a cell or a tissue of a mammal. The instructional material of the kit of the presently disclosed subject matter may, for example, be affixed to a container which contains the identified compound presently disclosed subject matter or be shipped together with a container which contains the identified compound. Alternatively, the instructional material may be shipped separately from the container with the intention that the instructional material and the compound be used cooperatively by the recipient.

An “isolated nucleic acid” refers to a nucleic acid segment or fragment which has been separated from sequences which flank it in a naturally occurring state, e.g., a DNA fragment which has been removed from the sequences which are normally adjacent to the fragment, e.g., the sequences adjacent to the fragment in a genome in which it naturally occurs. The term also applies to nucleic acids which have been substantially purified from other components which naturally accompany the nucleic acid, e.g., RNA or DNA or proteins, which naturally accompany it in the cell. The term therefore includes, for example, a recombinant DNA which is incorporated into a vector, into an autonomously replicating plasmid or virus, or into the genomic DNA of a prokaryote or eukaryote, or which exists as a separate molecule (e.g., as a cDNA or a genomic or cDNA fragment produced by PCR or restriction enzyme digestion) independent of other sequences. It also includes a recombinant DNA which is part of a hybrid gene encoding additional polypeptide sequence.

As used herein, a “ligand” is a compound that specifically binds to a target compound or molecule. A ligand “specifically binds to” or “is specifically reactive with” a compound when the ligand functions in a binding reaction which is determinative of the presence of the compound in a sample of heterogeneous compounds.

As used herein, the term “linkage” refers to a connection between two groups. The connection can be either covalent or non-covalent, including but not limited to ionic bonds, hydrogen bonding, and hydrophobic/hydrophilic interactions.

As used herein, the term “linker” refers to a molecule that joins two other molecules either covalently or noncovalently, such as but not limited to, through ionic or hydrogen bonds or van der Waals interactions.

The term “measuring the level of expression” or “determining the level of expression” as used herein refers to any measure or assay which can be used to correlate the results of the assay with the level of expression of a gene or protein of interest. Such assays include measuring the level of mRNA, protein levels, etc, and can be performed by assays such as northern and western blot analyses, binding assays, immunoblots, etc. The level of expression can include rates of expression and can be measured in terms of the actual amount of an mRNA or protein present. Such assays are coupled with processes or systems to store and process information and to help quantify levels, signals, etc, and to digitize the information for use in comparing levels

The term “nasal administration” in all its grammatical forms refers to administration of at least one compound of the presently disclosed subject matter through the nasal mucous membrane to the bloodstream for systemic delivery of at least one compound of the presently disclosed subject matter. The advantages of nasal administration for delivery are that it does not require injection using a syringe and needle, it avoids necrosis that can accompany intramuscular administration of drugs, trans-mucosal administration of a drug is highly amenable to self administration, and intranasal administration of antigens exposes the antigen to a mucosal compartment rich in surrounding lymphoid tissues, which can promote the development of a more potent immune response, particularly more potent mucosal immune responses.

The term “nucleic acid” typically refers to large polynucleotides. By “nucleic acid” is meant any nucleic acid, whether composed of deoxyribonucleosides or ribonucleosides, and whether composed of phosphodiester linkages or modified linkages such as phosphotriester, phosphoramidate, siloxane, carbonate, carboxymethylester, acetamidate, carbamate, thioether, bridged phosphoramidate, bridged methylene phosphonate, bridged phosphoramidate, bridged phosphoramidate, bridged methylene phosphonate, phosphorothioate, methylphosphonate, phosphorodithioate, bridged phosphorothioate or sulfone linkages, and combinations of such linkages. The term nucleic acid also specifically includes nucleic acids composed of bases other than the five biologically occurring bases (adenine, guanine, thymine, cytosine and uracil).

As used herein, the term “nucleic acid” encompasses RNA as well as single and double-stranded DNA and cDNA. Furthermore, the terms. “nucleic acid”. “DNA”. “RNA” and similar terms also include nucleic acid analogs, i.e., analogs having other than a phosphodiester backbone. For example, the so-called “peptide nucleic acids”, which are known in the art and have peptide bonds instead of phosphodiester bonds in the backbone, are considered within the scope of the presently disclosed subject matter.

By “nucleic acid” is meant any nucleic acid, whether composed of deoxyribonucleosides or ribonucleosides, and whether composed of phosphodiester linkages or modified linkages such as phosphotriester, phosphoramidate, siloxane, carbonate, carboxymethylester, acetamidate, carbamate, thioether, bridged phosphoramidate, bridged methylene phosphonate, bridged phosphoramidate, bridged phosphoramidate, bridged methylene phosphonate, phosphorothioate, methylphosphonate, phosphorodithioate, bridged phosphorothioate or sulfone linkages, and combinations of such linkages. The term nucleic acid also specifically includes nucleic acids composed of bases other than the five biologically occurring bases (adenine, guanine, thymine, cytosine and uracil). Conventional notation is used herein to describe polynucleotide sequences: the left-hand end of a single-stranded polynucleotide sequence is the 5′-end: the left-hand direction of a double-stranded polynucleotide sequence is referred to as the 5′-direction. The direction of 5′ to 3′ addition of nucleotides to nascent RNA transcripts is referred to as the transcription direction. The DNA strand having the same sequence as an mRNA is referred to as the “coding strand;” sequences on the DNA strand which are located 5′ to a reference point on the DNA are referred to as “upstream sequences”: sequences on the DNA strand which are 3′ to a reference point on the DNA are referred to as “downstream sequences”.

The term “nucleic acid construct”, as used herein, encompasses DNA and RNA sequences encoding the particular gene or gene fragment desired, whether obtained by genomic or synthetic methods.

Unless otherwise specified, a “nucleotide sequence encoding an amino acid sequence” includes all nucleotide sequences that are degenerate versions of each other and that encode the same amino acid sequence. Nucleotide sequences that encode proteins and RNA may include introns.

The term “oligonucleotide” typically refers to short polynucleotides, generally, no greater than about 50 nucleotides. It will be understood that when a nucleotide sequence is represented by a DNA sequence (i.e., A, T, G, C), this also includes an RNA sequence (i.e., A, U, G, C) in which “U” replaces “T”.

By describing two polynucleotides as “operably linked” is meant that a single-stranded or double-stranded nucleic acid moiety comprises the two polynucleotides arranged within the nucleic acid moiety in such a manner that at least one of the two polynucleotides is able to exert a physiological effect by which it is characterized upon the other. By way of example, a promoter operably linked to the coding region of a gene is able to promote transcription of the coding region.

The term “otherwise identical sample”, as used herein, refers to a sample similar to a first sample, that is, it is obtained in the same manner from the same subject from the same tissue or fluid, or it refers a similar sample obtained from a different subject. The term “otherwise identical sample from an unaffected subject” refers to a sample obtained from a subject not known to have the disease or disorder being examined. The sample may of course be a standard sample. By analogy, the term “otherwise identical” can also be used regarding regions or tissues in a subject or in an unaffected subject.

As used herein. “parenteral administration” of a pharmaceutical composition includes any route of administration characterized by physical breaching of a tissue of a subject and administration of the pharmaceutical composition through the breach in the tissue. Parenteral administration thus includes, but is not limited to, administration of a pharmaceutical composition by injection of the composition, by application of the composition through a surgical incision, by application of the composition through a tissue-penetrating non-surgical wound, and the like. In particular, parenteral administration is contemplated to include, but is not limited to, subcutaneous, intraperitoneal, intramuscular, intrasternal injection, and kidney dialytic infusion techniques.

The term “peptide” typically refers to short polypeptides but when used in the context of a longer amino acid sequence can also refer to a longer polypeptide.

The term “per application” as used herein refers to administration of a drug or compound to a subject.

The term “pharmaceutical composition” shall mean a composition comprising at least one active ingredient, whereby the composition is amenable to investigation for a specified, efficacious outcome in a mammal (for example, without limitation, a human). Those of ordinary skill in the art will understand and appreciate the techniques appropriate for determining whether an active ingredient has a desired efficacious outcome based upon the needs of the artisan.

As used herein, the term “pharmaceutically-acceptable carrier” means a chemical composition with which an appropriate compound or derivative can be combined and which, following the combination, can be used to administer the appropriate compound to a subject.

As used herein, the term “physiologically acceptable” ester or salt means an ester or salt form of the active ingredient which is compatible with any other ingredients of the pharmaceutical composition, which is not deleterious to the subject to which the composition is to be administered.

“Pharmaceutically acceptable” means physiologically tolerable, for either human or veterinary application.

As used herein. “pharmaceutical compositions” include formulations for human and veterinary use.

“Plurality” means at least two.

“Polypeptide” refers to a polymer composed of amino acid residues, related naturally occurring structural variants, and synthetic non-naturally occurring analogs thereof linked via peptide bonds, related naturally occurring structural variants, and synthetic non-naturally occurring analogs thereof.

“Synthetic peptides or polypeptides” means a non-naturally occurring peptide or polypeptide. Synthetic peptides or polypeptides can be synthesized, for example, using an automated polypeptide synthesizer. Various solid phase peptide synthesis methods are known to those of skill in the art.

By “presensitization” is meant pre-administration of at least one innate immune system stimulator prior to challenge with an agent. This is sometimes referred to as induction of tolerance.

The term “prevent”, as used herein, means to stop something from happening, or taking advance measures against something possible or probable from happening. In the context of medicine. “prevention” generally refers to action taken to decrease the chance of getting a disease or condition.

A “preventive” or “prophylactic” treatment is a treatment administered to a subject who does not exhibit signs, or exhibits only early signs, of a disease or disorder. A prophylactic or preventative treatment is administered for the purpose of decreasing the risk of developing pathology associated with developing the disease or disorder.

“Primer” refers to a polynucleotide that is capable of specifically hybridizing to a designated polynucleotide template and providing a point of initiation for synthesis of a complementary polynucleotide. Such synthesis occurs when the polynucleotide primer is placed under conditions in which synthesis is induced, i.e., in the presence of nucleotides, a complementary polynucleotide template, and an agent for polymerization such as DNA polymerase. A primer is typically single-stranded, but may be double-stranded. Primers are typically deoxyribonucleic acids, but a wide variety of synthetic and naturally occurring primers are useful for many applications. A primer is complementary to the template to which it is designed to hybridize to serve as a site for the initiation of synthesis, but need not reflect the exact sequence of the template. In such a case, specific hybridization of the primer to the template depends on the stringency of the hybridization conditions. Primers can be labeled with, e.g., chromogenic, radioactive, or fluorescent moieties and used as detectable moieties.

As used herein, the term “promoter/regulatory sequence” means a nucleic acid sequence which is required for expression of a gene product operably linked to the promoter/regulator sequence. In some instances, this sequence may be the core promoter sequence and in other instances, this sequence may also include other regulatory elements which are required for expression of the gene product. The promoter/regulatory sequence may, for example, be one which expresses the gene product in a tissue specific manner.

A “constitutive” promoter is a promoter which drives expression of a gene to which it is operably linked, in a constant manner in a cell. By way of example, promoters which drive expression of cellular housekeeping genes are considered to be constitutive promoters.

An “inducible” promoter is a nucleotide sequence which, when operably linked with a polynucleotide which encodes or specifies a gene product, causes the gene product to be produced in a living cell substantially only when an inducer which corresponds to the promoter is present in the cell.

A “tissue-specific” promoter is a nucleotide sequence which, when operably linked with a polynucleotide which encodes or specifies a gene product, causes the gene product to be produced in a living cell substantially only if the cell is a cell of the tissue type corresponding to the promoter.

A “prophylactic” treatment is a treatment administered to a subject who does not exhibit signs of a disease or exhibits only early signs of the disease for the purpose of decreasing the risk of contracting the disease and/or developing a pathology associated with the disease.

As used herein. “protecting group” with respect to a terminal amino group refers to a terminal amino group of a peptide, which terminal amino group is coupled with any of various amino-terminal protecting groups traditionally employed in peptide synthesis. Such protecting groups include, for example, acyl protecting groups such as formyl, acetyl, benzoyl, trifluoroacetyl, succinyl, and methoxysuccinyl: aromatic urethane protecting groups such as benzyloxycarbonyl; and aliphatic urethane protecting groups, for example, tert-butoxycarbonyl or adamantyloxycarbonyl. See Gross & Mienhofer, 1981 for suitable protecting groups.

As used herein. “protecting group” with respect to a terminal carboxy group refers to a terminal carboxyl group of a peptide, which terminal carboxyl group is coupled with any of various carboxyl-terminal protecting groups. Such protecting groups include, for example, tert-butyl, benzyl or other acceptable groups linked to the terminal carboxyl group through an ester or ether bond.

The term “protein” typically refers to large polypeptides. Conventional notation is used herein to portray polypeptide sequences: the left-hand end of a polypeptide sequence is the amino-terminus; the right-hand end of a polypeptide sequence is the carboxyl-terminus.

As used herein, the term “purified” and like terms relate to an enrichment of a molecule or compound relative to other components normally associated with the molecule or compound in a native environment. The term “purified” does not necessarily indicate that complete purity of the particular molecule has been achieved during the process.

A “highly purified” compound as used herein refers to a compound that is greater than 90% pure.

“Recombinant polynucleotide” refers to a polynucleotide having sequences that are not naturally joined together. An amplified or assembled recombinant polynucleotide may be included in a suitable vector, and the vector can be used to transform a suitable host cell.

A recombinant polynucleotide may serve a non-coding function (e.g., promoter, origin of replication, ribosome-binding site, etc.) as well.

A host cell that comprises a recombinant polynucleotide is referred to as a “recombinant host cell”. A gene which is expressed in a recombinant host cell wherein the gene comprises a recombinant polynucleotide, produces a “recombinant polypeptide”.

A “recombinant polypeptide” is one which is produced upon expression of a recombinant polynucleotide.

As used herein, the term “reporter gene” means a gene, the expression of which can be detected using a known method. By way of example, the Escherichia coli lacZ gene may be used as a reporter gene in a medium because expression of the lacZ gene can be detected using known methods by adding the chromogenic substrate o-nitrophenyl-β-galactoside to the medium (Gerhardt et al., 1994).

A “sample”, as used herein, refers preferably to a biological sample from a subject, including, but not limited to, normal tissue samples, diseased tissue samples, biopsies, blood, saliva, feces, semen, tears, and urine. A sample can also be any other source of material obtained from a subject which contains cells, tissues, or fluid of interest. A sample can also be obtained from cell or tissue culture.

By the term “specifically binds to”, as used herein, is meant when a compound or ligand functions in a binding reaction or assay conditions which is determinative of the presence of the compound in a sample of heterogeneous compounds.

The term “standard”, as used herein, refers to something used for comparison. For example, it can be a known standard agent or compound which is administered and used for comparing results when administering a test compound, or it can be a standard parameter or function which is measured to obtain a control value when measuring an effect of an agent or compound on a parameter or function. Standard can also refer to an “internal standard”, such as an agent or compound which is added at known amounts to a sample and is useful in determining such things as purification or recovery rates when a sample is processed or subjected to purification or extraction procedures before a marker of interest is measured. Internal standards are often a purified marker of interest which has been labeled, such as with a radioactive isotope, allowing it to be distinguished from an endogenous marker.

A “subject” of analysis, diagnosis, or treatment is an animal. Such animals include mammals, preferably a human.

As used herein, a “subject in need thereof” is a patient, animal, mammal, or human, who will benefit from the method of this presently disclosed subject matter.

As used herein. “substantially homologous amino acid sequences” includes those amino acid sequences which have at least about 95% homology, preferably at least about 96% homology, more preferably at least about 97% homology, even more preferably at least about 98% homology, and most preferably at least about 99% or more homology to an amino acid sequence of a reference antibody chain. Amino acid sequence similarity or identity can be computed by using the BLASTP and TBLASTN programs which employ the BLAST (basic local alignment search tool) 2.0.14 algorithm. The default settings used for these programs are suitable for identifying substantially similar amino acid sequences for purposes of the presently disclosed subject matter.

“Substantially homologous nucleic acid sequence” means a nucleic acid sequence corresponding to a reference nucleic acid sequence wherein the corresponding sequence encodes a peptide having substantially the same structure and function as the peptide encoded by the reference nucleic acid sequence: e.g., where only changes in amino acids not significantly affecting the peptide function occur. In some embodiments, the substantially identical nucleic acid sequence encodes the peptide encoded by the reference nucleic acid sequence. The percentage of identity between the substantially similar nucleic acid sequence and the reference nucleic acid sequence is in some embodiments at least about 50%, 65%, 75%, 85%, 95%, 99% or more. Substantial identity of nucleic acid sequences can be determined by comparing the sequence identity of two sequences, for example by physical/chemical methods (i.e., hybridization) or by sequence alignment via computer algorithm. Suitable nucleic acid hybridization conditions to determine if a nucleotide sequence is substantially similar to a reference nucleotide sequence are: 7% sodium dodecyl sulfate SDS, 0.5 M NaPO₄, 1 mM EDTA at 50° C., with washing in 2× standard saline citrate (SSC). 0.1% SDS at 50° C.; preferably in 7% (SDS), 0.5 M NaPO₄, 1 mM EDTA at 50° C., with washing in 1×SSC. 0.1% SDS at 50° C.; preferably 7% SDS, 0.5 M NaPO₄, 1 mM EDTA at 50° C., with washing in 0.5×SSC. 0.1% SDS at 50° C.; and more preferably in 7% SDS, 0.5 M NaPO₄, 1 mM EDTA at 50° C., with washing in 0.1×SSC. 0.1% SDS at 65° C. Suitable computer algorithms to determine substantial similarity between two nucleic acid sequences include. GCS program package (Devereux et al., 1984), and the BLASTN or FASTA programs (Altschul et al., 1990b; Altschul et al., 1990a; Altschul et al., 1997). The default settings provided with these programs are suitable for determining substantial similarity of nucleic acid sequences for purposes of the presently disclosed subject matter.

The term “substantially pure” describes a compound, e.g., a protein or polypeptide which has been separated from components which naturally accompany it. Typically, a compound is substantially pure when it is in some embodiments at least 10%, in some embodiments at least 20%, in some embodiments at least 50%, in some embodiments at least 60%, in some embodiments at least 75%, in some embodiments at least 90%, and in some embodiments at least 99% of the total material (by volume, by wet or dry weight, or by mole percent or mole fraction) in a sample is the compound of interest. Purity can be measured by any appropriate method, e.g., in the case of polypeptides by column chromatography, gel electrophoresis, or HPLC analysis. A compound, e.g., a protein, is also substantially purified when it is essentially free of naturally associated components or when it is separated from the native contaminants which accompany it in its natural state.

The term “symptom”, as used herein, refers to any morbid phenomenon or departure from the normal in structure, function, or sensation, experienced by the patient and indicative of disease. In contrast, a “sign” is objective evidence of disease. For example, a bloody nose is a sign. It is evident to the patient, doctor, nurse and other observers.

A “therapeutic” treatment is a treatment administered to a subject who exhibits signs of pathology for the purpose of diminishing or eliminating those signs.

A “therapeutically effective amount” of a compound is that amount of compound which is sufficient to provide a beneficial effect to the subject to which the compound is administered.

The term to “treat”, as used herein, means reducing the frequency with which symptoms are experienced by a patient or subject or administering an agent or compound to reduce the frequency with which symptoms are experienced.

A “prophylactic” treatment is a treatment administered to a subject who does not exhibit signs of a disease or exhibits only early signs of the disease for the purpose of decreasing the risk of developing pathology associated with the disease.

By the term “vaccine”, as used herein, is meant a composition which when inoculated into a subject has the effect of stimulating an immune response in the subject, which serves to fully or partially treat and/or protect the subject against a condition, disease or its symptoms. The term vaccine encompasses prophylactic as well as therapeutic vaccines. A combination vaccine is one which combines two or more vaccines, or two or more compounds or agents.

A “vector” is a composition of matter which comprises an isolated nucleic acid and which can be used to deliver the isolated nucleic acid to the interior of a cell. Numerous vectors are known in the art including, but not limited to, linear polynucleotides, polynucleotides associated with ionic or amphiphilic compounds, plasmids, and viruses. Thus, the term “vector” includes an autonomously replicating plasmid or a virus. The term should also be construed to include non-plasmid and non-viral compounds which facilitate transfer or delivery of nucleic acid to cells, such as, for example, polylysine compounds, liposomes, and the like. Examples of viral vectors include, but are not limited to, adenoviral vectors, adeno-associated virus vectors, retroviral vectors, recombinant viral vectors, and the like. Examples of non-viral vectors include, but are not limited to, liposomes, polyamine derivatives of DNA and the like.

“Expression vector” refers to a vector comprising a recombinant polynucleotide comprising expression control sequences operatively linked to a nucleotide sequence to be expressed. An expression vector comprises sufficient cis-acting elements for expression: other elements for expression can be supplied by the host cell or in an in vitro expression system. Expression vectors include all those known in the art, such as cosmids, plasmids (e.g., naked or contained in liposomes) and viruses that incorporate the recombinant polynucleotide.

### II. Representative Embodiments

It was previously showed that the fusion peptide of SARS-CoV-2 (here used as PSKPSKRSFIEDLLFNKVTLADAG (SEQ ID NO: 1)) or Porcine Epidemic Diarrhea Virus (PEDV; GRVVQKRSFIEDLLFNKVVTNGLG (SEQ ID NO: 5)) could be expressed on the surfaces of wild type and genome reduced E. coli, and make killed whole cell vaccines from those bacteria that protected pigs against clinical disease when the pigs were challenged with live PEDV (Maeda et al., 2021).

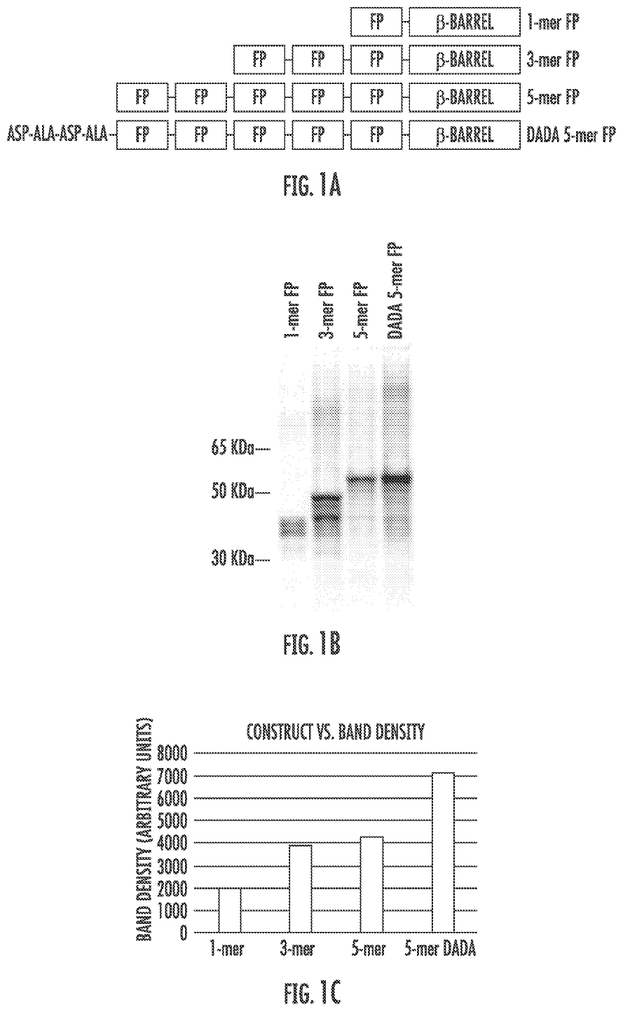

In some embodiments, the presently disclosed subject matter employs an antigen is a tandem repeat (3-mer. 5-mer, and DADA-5-mer) of the antigen (e.g., a fragment of the spike protein referred to herein as the fusion peptide, which in some embodiments comprises the amino acid sequence PSKPSKRSFIEDLLFNKVTLADAG (SEQ ID NO: 1); SEQ ID NO: 1) expressed on the bacterial surface. In some embodiments, the presently disclosed subject matter expresses a 3-mer concatemer and a 5-mer concatemer of the SARS-CoV-2 fusion peptide (see FIG. 1). It is further shown that adding an additional asp-ala-asp-ala (DADA) peptide to the recombinant fusion peptide (e.g., at the N-terminus of the peptide) expressed using the autotransporter (DADA-5-mer) substantially enhanced expression of the concatemer and minimizes faulty processing and proteolysis.

Thus, in some embodiments, the compositions and methods of the presently disclosed subject matter employ an expression enhancer, such as a single amino acid or an expression enhancer peptide, which in some embodiments is a dipeptide, tripeptide, or a concatemer of one or more dipeptides or tripeptides. An exemplary expression enhancer peptide for use in the compositions and methods of the presently disclosed subject matter is the dipeptide Asp-Ala (DA), which is some embodiments is dimerized to the tetrapeptide Asp-Ala-Asp-Ala (DADA). It is noted, however, that the dipeptide DA and the tetrapeptide DADA are merely exemplary, and that conservative amino acid substitutions of the Asp or the Ala are also employable within the scope of the presently disclosed subject matter, as disclosed herein below. Further, the expression enhancer could be a single amino acid, such as a single polar amino acid, or two, three, four or more polar amino acids, without the intervening ala or other amino acid. That is, in some embodiments, each position of a particular expression enhancer peptide can be a polar amino acid (e.g., consecutive polar amino acids), including the same polar amino acid or any combination of polar amino acids in a given sequence. Representative polar amino acids are described herein below and can include but are not necessarily limited to Ser, Asp, Asn, Glu, Gln, His, Arg, and Lys, and any combination thereof.

In some embodiments, the presently disclosed subject matter provides a generally applicable method for enhancing expression of an antigen or other bioactive agent, referred to herein in some embodiments as a “passenger protein”. By way of example and not limitation, in some embodiments, an expression enhancer (e.g., an expression enhancer peptide) comprising one or more polar amino acid residues are added to the N-terminal of any expressed protein, such as hydrophobic recombinant passenger proteins, to get better expression. In one particular, non-limiting embodiment, an asp-ala-asp-ala (DADA) sequence is added to the N-terminal of any autotransporter-expressed protein, particularly a hydrophobic recombinant passenger protein, to get better expression, including expression on the surface of a bacterium. In some embodiments, the added expression enhancer (e.g., an expression enhancer peptide such as but not limited to DADA or the like) is employed with a passenger protein, such as but not limited to an antigen expressed via an autotransporter, but could also be employed with any protein expressed in any construct for any functional bioengineering goal.

The above-discussed expression enhancer peptide asp-ala-asp-ala (DADA) is just one example/implementation. A single or more than two DA can also be employed. Additional polar amino acids, with both acidic and basic side chains, can be substituted into the sequence, as further described herein.

In some embodiments, the presently disclosed subject matter can used in vaccine applications, including applications employing wild type and/or genome reduced killed whole cell vaccine applications. Thus, the presently disclosed subject matter can be used with either genome reduced or wild type bacteria. However, additional applications are provided. By way of example and not limitation, in some embodiments, the resulting bacteria with enhanced surface expression by the addition of the sequences disclosed herein can have many other uses besides vaccines, for example, as a starting material for the production of proteins/peptides that are very hydrophobic and could be cleaved off the surface of the bacteria, or for bacteria expressing proteins on their surfaces for other biotechnological purposes, such as the expression of enzymes and the resulting use of the bacteria as particles catalysts in bioprocesses.

FIG. 1 shows enhanced expression of a multimeric passenger protein in an autotransporter by addition of hydrophilic amino acids. The top of the figure shows schematic diagrams of the passenger proteins cloned into the AIDA-I expression cassette of pRAIDA2 (Maeda et al., 2021). Shown are 1-mer. 3-mer, 5-mer, and DADA-5-mer versions of the recombinant autotransporter in the expression cassette. The lower left panel shows an immunoblot (stained with an anti-fusion peptide rabbit polyclonal antibody) in which equal numbers of bacteria were loaded into each lane of the gel. The right lower panel of the figure shows the densitometric quantitation of the bands observed in the immunoblot. The 3-mer shows increased antigen expression compared to the 1-mer, and the 5-mer shows minimal increased expressed vs, the 5-mer. However, the DADA-5-mer shows an almost doubling of expression compared to plain 5-mer, and a much more homogeneous protein band, with much diminished evidence of proteolytic degradation or other side products. Sequences for the constructs are provided in the EXAMPLES herein below.

### Representative Vaccine Embodiments

A novel coronavirus, severe acute respiratory syndrome coronavirus 2 (SARS-CoV-2), emerged in Wuhan. China in December 2019 causing novel coronavirus disease (COVID-19), with a high mortality rate, characterized by respiratory disease, and diarrhea in some patients. A vaccine for COVID-19 is urgently needed. A desirable vaccine for global pandemic use, beyond being safe and effective, would target highly conserved neutralizing epitopes, be inexpensive to manufacture, drawing on widely available production technology, and readily adaptable for worldwide use.

Gram-negative bacterial autotransporter (ATs), are a protein family that enable bacteria to place proteins into the outer membrane (Kilpatrick et al., 1997; Shata et al., 2000; Berry et al., 2003; Ulmer et al., 2006), have 3 domains: an N-terminal signal sequence for transport across the inner membrane, a C-terminal β-barrel that inserts a pore-like structure into the outer membrane, and a central passenger protein domain that transits through the pore to be exposed extracellularly. ‘displaying’ the passenger protein to the environment. Sequence encoding a protein of interest can replace native passenger protein sequence, yielding recombinant ATs that display ˜2×105 foreign proteins on each cell (Ulmer et al., 2006). The Haemophilus influenzae Hia AT, a trimeric AT, has a structure that strongly resembles the those of class I fusion protein stalks (van Bloois et al., 2011; Nicolay et al., 2015).

One of the oldest vaccine technologies is the killed whole cell vaccine (KWCV). While some KWCVs have been replaced by newer technologies, they remain important, in part because they are inexpensive and easy to manufacture. KWCVs simply require that bacteria be grown, inactivated, concentrated, and packaged. Many developing countries produce KWCVs indigenously. KWCVs are currently licensed to prevent deadly diseases, for example cholera (Bi et al., 2017). A description of how 6 million doses of WHO-prequalified Euvichol oral cholera vaccine were produced in 1 year using a single 100 L bioreactor for <$1/dose further highlights KWCV's advantages (Odevall et al., 2018). A globally useful vaccine should be very inexpensive, and quickly and easily manufactured, particularly when vaccines are urgently needed in response to pandemics.

The Tokyo Metropolitan University Group (Hashimoto et al., 2005; Kato & Hashimoto. 2007) made a systematic set of deletions in the E. coli genome and showed that they can delete 29.7% of the genome, yet retain a viable, albeit slow growing organism (Hashimoto et al., 2005; Kato & Hashimoto, 2007).

Disclosed herein is a globally appropriate SARS-CoV-2 candidate vaccine. As disclosed herein, in some embodiments the presently disclosed subject matter relates to construction of recombinant bacterial live and/or killed whole cell coronavirus (e.g., SARS-CoV, SARS-CoV-2, and/or PEDV) vaccines by displaying the spike protein stalk on the surfaces of E. coli using trimeric Gram-negative autotransporters (ATs). The coronavirus S protein is a class I viral fusion protein, similar to the HIV Env, Ebola gp, and influenza HA. The S protein stalk is a structural cognate of virion env proteins (e.g. HIV membrane-proximal external region. MPER, influenza HA stalk) targeted by broadly neutralizing mAbs. AT expression cassettes can place up to 105 recombinant antigens on the surface of each cell.

As disclosed herein, fusion peptides (FPs) of coronavirus spike proteins are exemplary targets for vaccine development since they are essential for virus entry. The FP is highly conserved among all coronaviruses. As disclosed herein, displaying SARS-CoV-2 FP on the surface of E. coli results in an effective vaccine against SARS-CoV-2. DNAs encoding the FPs of coronavirus (e.g., SARS-CoV and/or SARS-CoV-2) can be synthesized as a can be synthesized as a 1-mer or a homo- or hetero-concatemerized multimer (e.g., 3-mer concatemer and a 5-mer concatemer) each connected by a linker (e.g., a peptide linker, optionally a glycine linker, further optionally a glycine linker with the sequence FGGG (SEQ ID NO: 87), GGGF (SEQ ID NO: 88), SGGG (SEQ ID NO: 89), GGGS (SEQ ID NO: 90), or any combination thereof) to express FP on surfaces of bacteria using an AT expression cassette. Surface expression can be confirmed, for example, by flow cytometry. The recombinant bacteria can be inactivated, for example, by formalin to produce a killed whole cell SARS-CoV-2 vaccine. Finally, immunogenicity studies can be conducted to assess vaccine immunogenicity by determining the frequency of antigen-specific T cell response, neutralizing antibody, and neutralizing responses, and T-cell proliferation responses to the antigen.

Thus, in some embodiments, the presently disclosed subject matter provides 3-mers and 5-mers of the SARS-CoV-2 FP. However, the presently disclosed subject matter further provides concatemerized multimers of FPs from multiple different coronaviruses so as to broaden responses as much as possible, for the ultimate goal of making a universal coronavirus. This can also be done for other FPs and indeed for other multimers of other peptides as would be apparent to one of ordinary skill in the art upon a review of the instant disclosure, including for other viruses or other antigen targets. Thus, again, addition of an expression enhancer of the presently disclosed subject matter (e.g., expression enhancer peptide comprising a DADA sequence or the like) at the end of the expressed protein/peptide is generally applicable to any protein/peptide expressed using an expression vector as disclosed herein, including but not limited to an autotransporter. Also, the presently disclosed subject matter can employ any autotransporter, not just AIDA-I, and not just monomeric autotransporter, but also including but not limited to trimeric autotransporters.

### Genome Reduced Bacteria

The presently disclosed subject matter relates in some embodiments to the effects on immunogenicity of expressing immunogens, such as vaccine antigens, in bacteria have a reduced or eliminated expression of genes. Thus, in some embodiments, the bacterium with fewer expressed genes is more immunogenic. By way of example and not limitation, wholesale reduction of the bacterial genome, by means of small or large scale deletions, is one way this might be accomplished. Other approaches for decreasing expression of one or more genes are contemplated to fall within the scope of the presently disclosed subject matter, such as but not limited to specific knock outs, targeted inactivations or excisions by any one of several approaches (exemplary, but not exclusively through CRISPR/Cas9, TALENS, ZFNs), knock downs, effects on promoters, conditional mutants and/or inducible mutants (for use in better growing up the bacteria that may be growth restricted by the mutations or gene inactivations, or in live attenuated bacterial vaccines. In some representative, non-limiting embodiments, genes affecting surface structures can be affected. Expression of protein structures can be affected, as can non-protein structures.

In accordance with some embodiments of the presently disclosed subject matter, the terms “genome reduced” “genome reduction” or “GR” are used interchangeably and encompasses actual deletions but also other modifications, such as inactivation, functional inactivation, and/or mutation, which reduce expression of one or more genes. In some embodiments, reducing and/or eliminating expression of genes in the bacteria yields the enhanced immunogenicity. In some embodiments, the reduced number of expressed genes comprises a reduction of at least about 1.0%, 2.0%, 3.0%, 4.0%, 5.0%, 6.0%, 7.0%, 8.0%, 9.0%, 10%, 11%, 12%, 13%, 14%, 15%, or greater than 15% of genes. In some embodiments, the reduced number of expressed genes comprises a reduction of expressed genes selected from the group consisting of at least about 2.4%, at least about 15.9%, and at least about 29.7%.

In some embodiments of the presently disclosed subject matter, there is a steady increase in immunogenicity as more and more genes are deleted, without a distinct “threshold effect” or notable discontinuity, which supports that beyond the effects of deleting a specific gene, there are effects due to the overall quantitative reduction in the number of genes.

Genes may be completely or partially deleted, for example by the methods employed by Hashimoto et al., 2005 and by the lambda Red systems described by Datsenko & Wanner, 2000; by CRISPR/Cas9; and other methods to delete, inactivate, or decrease expression of bacteria genes as would be apparent to one of ordinary skill in the art upon a review of the instant disclosure. In some embodiments, bacteria that have a reduced expression of a set of genes and that have an immunogen of interest, such as but not limited to on their surfaces, elicit an enhanced immune response against that immunogen compared to wild type, non-gene reduced bacteria.

### Expression Vectors

In some embodiments, an expression vector comprising a nucleotide sequence encoding an expression enhancer (e.g., an expression enhancer single amino acid or an expression enhancer peptide) and a nucleotide sequence encoding a passenger protein, such as an antigen, is provided operably linked as described herein. In some embodiments, the expression vector is configured to express the passenger protein in a bacterium modified by transformation with the vector as described herein. The bacterium can be a wild type bacterium, a genome reduced bacterium, or bacterium having another modification in addition to be transformed by the expression cassette. The presently disclosed subject matter encompasses any suitable expression vector as would be apparent to one of ordinary skill in the art upon a review of the instant disclosure. In some embodiments, the passenger protein is expressed on the surface of the bacterium. In some embodiments, the vector comprises an autotransporter (AT) expression vector. In some embodiments, the vector comprises a codon optimized sequence encoding the antigen. In some embodiments, the AT expression vector comprises a monomeric vector or a trimeric vector. In some embodiments, the nucleotide sequence encoding the antigen is positioned under control of an inducible promoter or a constitutive promoter. In some embodiments, the passenger protein is expressed as a monomer or as a trimer. In some embodiments, the vector is provided in a pharmaceutically acceptable carrier. Thus, one way the passenger protein can be placed on the surface of the bacteria is using an autotransporter (monomeric or trimeric), by itself, or in the context of a foreign protein or scaffold to enhance/improve formation of desired passenger protein. The autotransporter expression is not the only way to express antigens. Other vectors can be employed. In further embodiments, passenger proteins can be placed on the surface of the bacteria using other technologies, for example by covalent or non-covalent linkage, absorption, affinity tag, and the like. Using other technologies to place passenger proteins on the surfaces of the bacteria provides for the production of constructs and/or vaccines directed against proteins or other antigens that cannot be expressed on the bacterial surface using expression cassettes or against non-protein antigens (such as but not limited to polysaccharides).

There are many other ways to express antigens and/or to specifically place them on the surfaces of the bacteria, or even inside the bacteria, such as but not limited to covalent coupling of the antigen to the surface of the bacteria, association of the bacteria with antigen non-covalently using an affinity tag, non-specific adsorption, addition of a binding moiety to the antigen followed by mixing the antigen with the bacteria.

The expression cassette approach, e.g., autotransporter expression cassette approach, provides a synthetic biology solution: the protein antigen need not be isolated/purified/conjugated to carrier protein. Only the identity of the protein is needed. Then the coding sequence can be rapidly synthesized and cloned into the appropriate expression vector, followed by expression in the wild type or GR bacteria.

The wild type/native protein can be used, or a component of the protein can be used, if it is desirable to produce an immune response only against a particular component of the protein. A mutated version of the protein can be used, to enhance immune responses or to bias immune responses (in a non-exclusive example, humoral vs, cellular). The antigen or immunogen, used interchangeably herein, can be used to elicit an immune response against a pathogen, as in developing a prophylactic vaccine. The immunogen can be used to elicit an immune response against a pathogen, as in developing a therapeutic vaccine, for example to treat a chronic infectious disease, including chronic viral diseases. One example would be a coronavirus, for example SARS-CoV and/or SARS-CoV-2, in a coronavirus-infected patient (such as but not limited to a patient with SARS and/or COVID-19). In some embodiments, the antigen or immunogen can be an immunogenic fragment of a virus spike(S) protein, including but not limited to a sequences derived from a fusion peptide of a virus, such as but not limited to a fusion peptide of a coronavirus, for example SARS-CoV, e.g., SARS-CoV-2 FP. In some embodiments, the antigen or immunogen can be an immunogenic fragment of a virus spike(S) protein, including but not limited to a sequences derived from a fusion peptide of a virus, such as but not limited to a fusion peptide of a coronavirus, for example SARS-CoV-2.

All of the above prophylactic and therapeutic uses can be in humans or animals. For example, the technology can be used to make veterinary prophylactic infectious disease vaccines.

The immunogen can be used to elicit the rapid production of antibodies in animals for the purposes of producing antibodies. These can be, for example, custom polyclonal antibodies, obtained directly from various species used to make custom polyclonal antibodies, such as rabbits, goats, sheep, horses, cows, and camelidae. The antibodies can be obtained from serum or from colostrum.

The immunogen can be used to immunize animals (e.g., mice, but also other species, including rabbits) to accelerate the production of monoclonal antibodies, since the first step in making a monoclonal antibody is to immunize an animal so that it makes antibodies, so that its spleen cells can be fused with myeloma cells to make a hybridoma. Such monoclonal antibodies can be used in all the analytic, diagnostic, and therapeutic ways in which monoclonal antibodies are typically used.

The bacterial immunogen can be a killed/inactivated bacterium or a live bacterium. The bacteria can be killed/inactivated in many different ways: formalin, glutaraldehyde, heat, radiation, other chemicals. The bacteria can be whole bacteria or derivatives of whole bacteria, such as but not limited to minicells, ghost cells, blebs, vesicles. The vaccine could also be fragments of the cells, including genome reduced cells. Such derivatives are prepared in accordance with techniques recognized in the art, as would be apparent to one of ordinary skill in the art up on a review of the instant disclosure.

The bacterium can be any bacterium, including Gram-negative bacteria. E. coli are not the only wild type or genome reduced bacteria that can be used. Other Gram-negative bacteria can be used, and other wild type or genome reduced strains of other bacteria can be used, such as but not limited to wild type or genome reduced Salmonella or even Vibrio. Such genome reduced versions of other bacterial species are prepared in accordance with techniques recognized in the art, as would be apparent to one of ordinary skill in the art up on a review of the instant disclosure, and then use them to express immunogens, such as vaccine antigens. Thus, in some embodiments the bacteria are from Enterobacteriaceae, such as but not limited to Salmonella. Klebsiella. Shigella, Yersinia. In some embodiments, representative bacteria can be chosen via a systematic review of the taxonomic tree: and thus, can include all Proteobacteria.

In some embodiments, a wild type and/or a reduced genome bacterium comprises a recombinant antigen on its surface, which in some embodiments can be a coronavirus antigen, more particularly a SARS-CoV and/or SARS-CoV-2 and/or PEDV antigen, and even more particularly an antigen derived from the SARS-CoV-2 spike(S) polypeptide, which can be used to elicit useful immune responses against such an antigen. Such modified reduced genome bacteria can be used as prophylactic and/or therapeutic vaccines against SARS-CoV-2 and/or PEDV.

Thus, in some embodiments, the presently disclosed subject matter relates to strategies for the rapid production of better immunogens for the production of new vaccines, including prophylactic vaccines for infectious diseases (e.g., diseases associated with coronavirus infections, such as but not limited to SARS-CoV and/or SARS-CoV-2 and/or PEDV infections, including but not limited to COVID-19) of humans and animals. These strategies include:

1 Direct antigen production in a bacterial cell employing a synthetic biology approach in which the antigen of interest is expressed directly in the bacteria, directed by recombinant coding sequence. In some implementations, coronavirus—(e.g., SARS-CoV and/or SARS-CoV-2 and/or PEDV) derived antigens are placed on the cell surface of a wild type bacterium, a genome reduced bacterium, or other modified bacterium, and in some implementations Gram-negative autotransporter protein expression cassettes are used to place the antigen-of-interest on the bacterial cell surface. Instead of purifying the protein antigen and conjugating it to carrier protein, antigen coding sequences can be cloned into an expression cassette. In the Gram-negative autotransporter embodiment discussed herein, these autotransporters (Type 5 Secretion Systems) place the antigen on the cell surface as the vaccine immunogen. This obviates any need to isolate or synthesize the protein antigen, purify the antigen, couple the antigen to an appropriate carrier, and prepare a parental immunization, saving up to several weeks.

2. Use of genome reduced bacteria (such as, but not limited to E. coli) to express the antigen. In some embodiments, the bacteria are Gram-negative bacteria, and in some embodiments the Gram-negative bacteria are E. coli. In representative embodiments, surface expressed SARS-CoV-2-derived and/or PEDV-derived antigens would be more accessible to the immune system and elicit better immune responses by expressing the antigens, such as but not limited to vaccine antigens, in genome reduced bacteria, in some embodiments on the surfaces of genome reduced bacteria, in some embodiments Gram-negative bacteria, and in one example on the surfaces of genome reduced (GR) E. coli.

3. Mucosal immunization. As a representative, non-limiting route, intranasal immunization exposes M cells and dendritic cells directly to the immunogen, and the oropharyngeal mucosa has a large amount of lymphoid tissue, which produces enhanced immune responses to intranasally administered immunogens. However, the presently disclosed subject matter encompasses any route of administration as would be apparent to one of ordinary skill in the art upon a review of the instant disclosure, including but not limited to topical, oral, rectally, vaginally, intravenous, intramuscular, intra-arterial, intramedullary, intrathecal, intraventricular, transdermal, subcutaneous, intraperitoneal, enteral, sublingual, or in the case of a neoplasm, intratumorally.

4. Exponential increasing (exp-inc) immunization. In a representative, non-limiting embodiment, sequential, rapid exposure to increasing amounts of immunogen can yield enhanced immune responses, thought to occur because such immunogen exposure kinetics mimic the antigen exposure a host would experience in the face of a severe, poorly controlled infection, which would trigger an enhanced immune response. The immunogens discussed in this application can be used in exp-inc immunization regimens to enhance immune responses against the antigen. Conversely, the immunogens described in this application can be used in exponential decreasing dose administration patterns, or at repeated low doses to elicit a tolerizing response.

Aspects of the presently disclosed subject matter relate at least in part to the use of genome reduced bacteria to produce an antigen capable rapidly inducing an immune response against an antigen. The antigen-expressing genome reduced bacteria enable rapid antibody production for use in making custom polyclonal antibodies and materials needed (for example plasma cells) for monoclonal antibodies. The antigen-expressing genome reduced bacteria also can serve as vaccine immunogens designed to elicit immune responses that protect against infectious agents.

As set forth herein, in some embodiments, expressing an antigen in a genome reduced bacterium can yield substantially higher binding of an antibody directed against the antigen to the bacteria and that bacteria expressing the test antigen elicit a significantly higher immune response against the test antigen when an animal is immunized with genome reduced bacteria expressing that test antigen than when immunized with wild type bacteria, and that bacteria with progressively increasing amounts of genome deletion elicited increasingly potent immune responses.

In one aspect of the presently disclosed subject matter, there is provided an expression cassette comprising a first coding sequence encoding an expression enhancement peptide, a second coding sequence encoding a polypeptide of interest, and a third coding sequence encoding a bacterial autotransporter β-barrel polypeptide, wherein (i) the first, second, and third coding sequences are in frame with each other such that transcription and translation of the expression cassette in a host cell produces a fusion protein comprising the expression enhancement peptide, the polypeptide of interest, and the bacterial autotransporter β-barrel polypeptide: (ii) the expression enhancement peptide comprises one or more copies of the amino acid sequence (X¹X²X³)ₙ, wherein each X¹, X², X³ is independently selected from the group consisting of Asp (D), Asn (N), Glu (E), Gln (Q), His (H), Arg (R), Lys (K); Ala (A), Ser(S), Thr (T), Pro (P), Gly (G), Met (M), Leu (L), Ile (I), Val (V), and Cys (C); each X³ is absent in any given member of the one or more copies of the amino acid sequence (X¹X²X³)ₙ or, if present, is independently any amino acid; and n is at least 1; and (iii) when expressed in a host cell, optionally a bacterium, further optionally a Gram-negative bacterium, the expression cassette induces expression of the polypeptide of interest on a surface of the host cell. In some embodiments, the expression cassette encodes at least one expression enhancer peptide attached to at least one polypeptide of interest attached to a bacterial autotransporter β-barrel polypeptide.

In some embodiments, the polypeptide of interest, optionally the antigen, is derived from a microbe, such as a pathogen. In some embodiments, the polypeptide of interest, optionally the antigen, is derived from a tumor and/or a cancer, a target of an inappropriate or undesirable immune response, or a component of the host immune system, optionally a host immune system component that, when targeted for destruction, inactivation, or activation, alters an undesirable immune response.

In some embodiments, the polypeptide of interest is a concatemer of two, three, four, five, or more copies of an amino acid sequence of interest, optionally wherein each of the two, three, four, five, or more copies of the amino acid sequence of interest are linked to each other via a peptide linker.

In some embodiments, the peptide linker comprises a peptide sequence selected from the group consisting of FGGG (SEQ ID NO: 87), GGGF (SEQ ID NO: 88), SGGG (SEQ ID NO: 89). GGGS (SEQ ID NO: 90), and any combination thereof. In some embodiments, at least one of the two or more copies of the amino acid sequence of interest in the concatemer comprises an expression enhancement peptide at its N-terminus, optionally wherein each of the two or more copies of the amino acid sequence of interest in the concatemer comprises an expression enhancement peptide at its N-terminus.

In some embodiments, the expression enhancement peptide comprises an amino acid sequence selected from the group consisting of DA, DS, DT, DP, DG, NA, NS, NT, NP, NG, EA, ES, ET, EP, QA, QG, QA, QS, QT, QP, QG, HA, HS, HT, HP, HG, RA, RS, RT, RP, RG, LA, LS, LT, LP, LG, DAX⁴, DSX⁴, DTX⁴, DPX⁴, DGX⁴, NAX⁴, NSX⁴, NTX⁴, NPX⁴, NGX⁴, EAX⁴, ESX⁴, ETX⁴. EPX⁴, QAX⁴, QGX⁴, QAX⁴, QSX⁴, QTX⁴, QPX⁴, QGX⁴, HAX⁴, HSX⁴, HTX⁴, HPX⁴, HGX⁴, RAX⁴. RSX⁴, RTX⁴, RPX⁴, RGX⁴, LAX⁴, LSX⁴, LTX⁴, LPX⁴, LGX⁴, DADA (SEQ ID NO: 11), EAEA (SEQ ID NO: 12), DAEA (SEQ ID NO: 13), EADA (SEQ ID NO: 14), NANA (SEQ ID NO: 15), QAQA (SEQ ID NO: 16), DANA (SEQ ID NO: 17), EANA (SEQ ID NO: 18), NADA (SEQ ID NO: 19), NAQA (SEQ ID NO: 20), HAHA (SEQ ID NO: 21). RARA (SEQ ID NO: 22), LALA (SEQ ID NO: 23), HSHS (SEQ ID NO: 24), HTHT (SEQ ID NO: 25), LSLS (SEQ ID NO: 26), LTLT (SEQ ID NO: 27), RSRS (SEQ ID NO: 28), RTRT (SEQ ID NO: 29), HPHP (SEQ ID NO: 30), HGHG (SEQ ID NO: 31), LPLP (SEQ ID NO: 32), LGLG (SEQ ID NO: 33), DSDS (SEQ ID NO: 34), ESES (SEQ ID NO: 35), DSES (SEQ ID NO: 36), ESDS (SEQ ID NO: 37), NSNS (SEQ ID NO: 38), QSQS (SEQ ID NO: 39), DSNS (SEQ ID NO: 40), ESNS (SEQ ID NO: 41), NSDS (SEQ ID NO: 42), NSQS (SEQ ID NO: 43), DTDT (SEQ ID NO: 44), ETET (SEQ ID NO: 45), DTET (SEQ ID NO: 46), ETDT (SEQ ID NO: 47), NTNT (SEQ ID NO: 48), QTQT (SEQ ID NO: 49), DTNT (SEQ ID NO: 50), ETNT (SEQ ID NO: 51), NTDT (SEQ ID NO: 52), NTQT (SEQ ID NO: 53), DPDP (SEQ ID NO: 54), EPEP (SEQ ID NO: 55), DPEP (SEQ ID NO: 56), EPDP (SEQ ID NO: 57), NPNP (SEQ ID NO: 58), QPQP (SEQ ID NO: 59), DPNP (SEQ ID NO: 60), EPNP (SEQ ID NO: 61), NPDP (SEQ ID NO: 62), NPQP (SEQ ID NO: 63), DGDG (SEQ ID NO: 64), EGEG (SEQ ID NO: 65), DGEG (SEQ ID NO: 66), EGDG (SEQ ID NO: 67), NGNG (SEQ ID NO: 68), QGQG (SEQ ID NO: 69), DGNG (SEQ ID NO: 70), EGNG (SEQ ID NO: 71), NGDG (SEQ ID NO: 72), NGQG (SEQ ID NO: 73), HAHS (SEQ ID NO: 74), RARS (SEQ ID NO: 75), LALS (SEQ ID NO: 76), HSHT (SEQ ID NO: 77), HTHP (SEQ ID NO: 78), LSLT (SEQ ID NO: 79), LTLP (SEQ ID NO: 80), RSRT (SEQ ID NO: 81), RTRP (SEQ ID NO: 82), HPHG (SEQ ID NO: 83), HGHA (SEQ ID NO: 84), LPLA (SEQ ID NO: 85), LGLA (SEQ ID NO: 86), and combinations thereof, wherein X⁴ is any amino acid.

In some embodiments, the presently disclosed subject matter provides an expression cassette comprising first coding sequence encoding an expression enhancement peptide, a second coding sequence encoding a polypeptide of interest, and a third coding sequence encoding a bacterial autotransporter-barrel polypeptide. The first, second, and third coding sequences are in frame with each other such that transcription and translation of the expression cassette in a host cell produces a fusion protein comprising the expression enhancement peptide, the polypeptide of interest, and the bacterial autotransporter β-barrel polypeptide. In some embodiments, the expression enhancement peptide can comprise 1, 2, 3, or more amino acids, polar, with or without glycines in between the polar amino acids. In some embodiments, the expression enhancement peptide can go up to ≥20. The expression enhancement peptide comprises one or more copies of the amino acid sequence (X¹X²X³)ₙ, wherein each X¹, X². X³ is independently selected from the group consisting of Asp (D), Asn (N), Glu (E), Gln (Q), His (H), Arg (R), Lys (K); Ala (A), Ser(S), Thr (T), Pro (P), Gly (G), Met (M), Leu (L), Ile (I), Val (V), and Cys (C); each X² and/or X³ is absent in any given member of the one or more copies of the amino acid sequence (X¹X²X³)ₙ or, if present, is independently any amino acid; and n is at least 1; and when expressed in a host cell, optionally a bacterium, further optionally a Gram-negative bacterium, the expression cassette induces expression of the polypeptide of interest on a surface of the host cell. In some embodiments, the definition X¹X²X³ includes optional embodiments comprising adding one or more polar amino acids to the N-terminus of the polypeptide of interest.

In some embodiments, the polypeptide of interest is an antigen, such as but not limited to an antigen designed to elicit an immune response: an antigen from a pathogen, such as but not limited to an antigen from a virus: an antigen from a biologically active molecule; an antigen from a molecule involved in an autoimmune process: an antigen against which is it desired to elicit an immune response to develop humor or cellular immunity; an antigen against which is it desired to elicit an immune response to develop humor or cellular immune responses to develop custom antibodies or custom cell mediated immune responses; an antigen from a cancer cell; and an antigen from SARS-CoV-2, HIV, PEDV, HIV etc.

In some embodiments, the polypeptide of interest includes an inflammatory molecules (e.g., TNF, INFg, ILs including 1, 2, 12, 18, GMCSF). In some embodiments, a polypeptide of interest includes a tumor antigen, such as a tumor neoantigen, e.g., antigens identified specific to a cancer cell by proteomics or sequencing. In some embodiments, a polypeptide of interest includes an immune checkpoint inhibitor, such as: CTLA4, PD-1, PD-L1(PD-1 ligand 1, or CD274), and PD-1 (also PDCD1 and CD279).

In some embodiments, the presently disclosed subject matter provides for the expression of an any antigen identified as being likely to be capable of eliciting a broadly neutralizing immune response, such as because it shows low or minimal diversity or evolution over many sequenced strains or is the binding target of broadly neutralizing monoclonal or polyclonal antibodies. In some embodiments, the presently disclosed subject matter provides for the expression of any antigen identified as being the binding site of a neutralizing monoclonal or polyclonal antibody, or antibody that is toxic for cancer cells, or affects a tissue, cell, or organ in a desired way. In embodiments, the presently disclosed subject matter provides for the expression of any antigen identified as being likely to be capable of eliciting a broadly neutralizing immune response because it shows low or minimal diversity or evolution over many sequenced strains. In some embodiments, the presently disclosed subject matter is applicable for all infections (including treatment and prophylaxis), all pathogens (including but not limited bacteria, viruses, veterinary pathogens, other agents consider not self and the like), cancer, targets for the treatment of autoimmune diseases, and commercial production of antibodies.

The presently disclosed subject matter provides in some embodiments expression on the surface of the bacteria of industrially useful molecules, such as enzymes, ligands, adhesins, and the like. Thus, in some embodiments, the presently disclosed subject matter provides for the expression of useful enzymes for industrial processes. In some embodiments, the polypeptide of interest thus includes include carbohydrases, proteases, and lipases. Amylases are in the carbohydrase group, plus cellulases, glucose isomerase, glucose oxidase, pectinases, xylanases, invertase, galactosidase. Proteases can include bromelain, subtilisin and others. Lipases can include any suitable lipase as would be apparent to one of ordinary skill in the art up on a review on the instant disclosure. Representative lipases include but are not limited to Candida antarctica lipase B (CALB), Candida rugosa lipase (CRL), PLA1s, PLA2s and PLBs extracted from A, oryzae and/or A. niger. See Chandra et al., Microbial Cell Factories, 19:169 (2020).

In some embodiments, a polypeptide of interest is a molecule of clinical or commercial importance. Exemplary clinically and/or commercially important molecules include, but are not limited to inflammatory molecules (such as but not limited to TNF, INFγ, interleukins (ILs) including but not limited to IL1, IL2, IL12, IL18, and GM-CSF), tumor- and/or cancer-associated antigens including but not limited to tumor neoantigens (e.g., antigens identified that are specific to a cancer cell by proteomics or sequencing), immune checkpoint inhibitors such as but not limited to CTLA4, PD-1, PD-L1 (also called CD274). PD-1 (also called PDCD1 and CD279); and lipases of industrial and/or commercial importance as those disclosed in Chandra et al. (2020) Microbial lipases and their industrial applications: a comprehensive review. Microbial Cell Factories 19: Article number 169. Exemplary such ligases include Candida antarctica lipase B (CALB), Candida rugosa lipase (CRL), and PLA1s, PLA2s and PLBs extracted from A, oryzae and A. niger.

Additional polypeptides of interest that can be employed in the compositions and methods of the presently disclosed subject matter include industrial enzymes such as but not limited to carbohydrases such as but not limited to amylases, cellulases, glucose isomerase, glucose oxidase, pectinases, xylanases, invertase, and galactosidase.

Proteases can also be employed as the polypeptides of interest in the compositions and methods of the presently disclosed subject matter, which non-limiting examples of such proteases including bromelain, subtilisin, and others.

In some embodiments, the presently disclosed subject matter provides for the expression of non-infectious antigens (in some embodiments, cancer antigens, including but not limited to VEGF, HER2, and EFGR): physiological important regulatory molecules (blood pressure renin angiotensin and clotting cascade and inflammatory cascade kallikrein-kinin); cancer neoantigens identified through the analysis of cancer cells, like proteomics, DNA sequencing, and RNA seq, and autoimmunity targets. In some embodiments, the presently disclosed subject matter provides for the expression of antigen for the prevention and treatment of infectious diseases (including but not limited viruses) in people and animals. These include antigens previously identified as being capable of eliciting protective immunity, and antigens identified because they are the targets of broadly neutralizing polyclonal and monoclonal antibodies, and antigens identified as being able to elicit broadly protective immunity because their genetic (RNA for RNA viruses or DNA for DNA viruses and other pathogens) sequences are stable over many sequenced isolates.

In some embodiments, the presently disclosed subject matter involves a genome reduced bacteria of any amount of genome reduction, including larger amounts. In some embodiments, the presently disclosed subject matter involves any viable bacteria, and particularly any bacteria with all genes encoding non-essential proteins and other substances on the surface of the bacteria deleted and any genome deletions that maintain viability of the bacteria.

In some embodiments, the presently disclosed subject matter employs 3 to 20 concatamers, including 3, 5, 10, or 20. In some embodiments, the presently disclosed subject matter provides bacteria expressing antigens as described here combined with different adjuvants.

In some embodiments, the polypeptide of interest is an antigen, optionally a viral antigen. In some embodiments, the viral antigen comprises a coronavirus antigen, optionally a severe acute respiratory syndrome-associated coronavirus (SARS-CoV) antigen, a severe acute respiratory syndrome-associated coronavirus 2 (SARS-CoV-2) antigen, and/or a porcine epidemic diarrhea virus (PEDV) antigen; or a human immunodeficiency virus (HIV) antigen.

In some embodiments, the viral antigen comprises (i) a coronavirus fusion peptide (FP) antigen, optionally a coronavirus FP antigen comprising an amino acid sequence selected from the group consisting of PSKPSKRSFIEDLLFNKVTLADAGF (SEQ ID NO: 2), PSKPSKRSFIEDLLENKKTLADAG (SEQ ID NO: 1), SFIEDLLF (SEQ ID NO: 4), SFIEDLLFNKVTLADAGF (SEQ ID NO: 3), GRVVQKRSFIEDLLFNKVVTNGLG (SEQ ID NO: 5), and immunogenic fragments thereof; or (ii) an HIV antigen comprising an amino acid sequence selected from the group consisting of AVGIGAVF (SEQ ID NO: 7), ALGIGAAF (SEQ ID NO: 8), AVGFGAAF (SEQ ID NO: 9), AAGFGAMF (SEQ ID NO: 10), and immunogenic fragments thereof; or (iii) any combination thereof.

In some embodiments, the expression vector is configured to express the polypeptide of interest in a genome reduced bacterium or derivative thereof, optionally on a surface of the genome reduced bacterium or derivative thereof. In some embodiments, one or more of the first, second, and third coding sequences comprises a codon optimized coding sequence.

In one aspect of the presently disclosed subject matter, there is provided an expression vector comprising the expression cassette described herein. In some embodiments, the expression cassette is operably linked to an inducible promoter or a constitutive promoter. In some embodiments, the expression vector, further comprising a pharmaceutically acceptable carrier, excipient, or diluent.

In one aspect of the presently disclosed subject matter, there is provided a host cell comprising the expression cassette and/or the expression vector as described herein. In some embodiments, the host cell comprises a bacterium. In some embodiments, the host cell comprises a Gram negative bacterium. In some embodiments, the host cell comprises an Escherichia coli. In some embodiments, the host cell comprises a Salmonella bacterium.

In some embodiments, the bacterium comprises a genome reduced bacterium. In some embodiments, the genome reduced bacterium is characterized by a reduced number of expressed genes comprises a reduction of expressed genes of at least about 1.0%, 2.0%, 3.0%, 4.0%, 5.0%, 6.0%, 7.0%, 8.0%, 9.0%, 10%, 11%, 12%, 13%, 14%, 15%, or greater than 15% of genes relative to a non-genome reduced bacterium upon which the genome reduced bacterium or derivative thereof is based.

In some embodiments, the reduced number of expressed genes comprises a reduction of expressed genes selected from the group consisting of at least about 2.4%, at least about 15.9%, and at least about 29.7% relative to a non-genome reduced bacterium upon which the genome reduced bacterium or derivative thereof is based. In some embodiments, the polypeptide of interest is expressed on a surface of the host cell.

### Representative Compositions and Methods

In one aspect of the presently disclosed subject matter, there is provided a vaccine composition comprising the expression cassette as disclosed herein, the expression vector as disclosed herein, the host cell as disclosed herein, and a pharmaceutically acceptable carrier, diluent, or excipient, one or more adjuvants, or any combination thereof. In some embodiments, the vaccine composition comprises a bacterium, optionally a genome reduced bacterium, and further wherein the bacterium is a live attenuated bacterium or a killed whole cell bacterium or an immunogenic fragment thereof that comprises the polypeptide of interest.

In some embodiments, the vaccine composition is formulated to be administered orally, rectally, vaginally, intra-nasally, parenterally, intradermally, subcutaneously, or intramuscularly. In some embodiments, the vaccine composition further comprises an adjuvant.

In some embodiments, the polypeptide of interest is an antigen, optionally a viral antigen. In some embodiments, the viral antigen comprises a coronavirus antigen, optionally a severe acute respiratory syndrome-associated coronavirus (SARS-CoV) antigen, a severe acute respiratory syndrome-associated coronavirus 2 (SARS-CoV-2) antigen, and/or a porcine epidemic diarrhea virus (PEDV) antigen; or a human immunodeficiency virus (HIV) antigen.

In some embodiments, the viral antigen comprises (i) a coronavirus fusion peptide (FP) antigen, optionally a coronavirus FP antigen comprising an amino acid sequence selected from the group consisting of PSKPSKRSFIEDLLFNKVTLADAGF (SEQ ID NO: 2), PSKPSKRSFIEDLLFNVTLADAG (SEQ ID NO: 1), SFIEDLLF (SEQ ID NO: 4), SFIEDLLFNKVTLADAGF (SEQ ID NO: 3), GRVVQKRSFIEDLLFNKVVINGLG (SEQ ID NO: 5), and immunogenic fragments thereof; or (ii) an HIV antigen comprising an amino acid sequence selected from the group consisting of AVGIGAVF (SEQ ID NO: 7), ALGIGAAF (SEQ ID NO: 8), AVGFGAAF (SEQ ID NO: 9), AAGFGAMF (SEQ ID NO: 10), and immunogenic fragments thereof; or (iii) any combination thereof.

In one aspect of the presently disclosed subject matter, there is provided a method for inducing an immune response in a subject. In some embodiments, the method comprises: administering to the subject a composition comprising, consisting essentially of, or consisting of the expression cassette as disclosed herein, the expression vector as disclosed herein, and/or the host cell as disclosed herein, in an amount and via a route sufficient to induce an immune response to the polypeptide of interest in the subject

In some embodiments, the immune response to the polypeptide of interest induced in the subject is enhanced relative to an immune response that would be induced in the subject when an expression cassette that is identical to the expression cassette as disclosed herein, an expression vector that is identical to the expression vector as disclosed herein, and/or a host cell as disclosed herein that is identical to the host cell as disclosed herein but that lacks the first coding sequence that encodes the expression enhancement peptide is administered to the subject in the same amount and via the same route.

In some embodiments, the composition is administered parenterally. In some embodiments, the composition is administered non-parenterally.

In one aspect of the presently disclosed subject matter, there is provided a method for enhancing expression of a polypeptide of interest in a host cell. In some embodiments, the method comprises modifying the polypeptide of interest to include an expression enhancement peptide moiety at or near its N-terminus, wherein the expression enhancement peptide comprises one or more copies of the amino acid sequence (X¹X²X³)ₙ, wherein each X¹ is independently selected from the group consisting of Asp (D), Asn (N), Glu (E), Gln (Q), His (H), Arg (R), and Lys (K); each X² is independently selected from the group consisting of Ala (A), Ser(S), Thr (T), Pro (P), Gly (G), Met (M), Leu (L), Ile (I), Val (V), and Cys (C); each X³ is absent in any given member of the one or more copies of the amino acid sequence (X¹X²X³)ₙ or, if present, is independently any amino acid; and n is at least 1.

In some embodiments, the expression enhancement peptide comprises an amino acid sequence selected from the group consisting of DA, DS, DT, DP, DG, NA, NS, NT, NP, NG, EA, ES, ET, EP, QA, QG, QA, QS, QT, QP, QG, HA, HS, HT, HP, HG, RA, RS, RT, RP, RG, LA, LS, LT, LP, LG, DAX⁴, DSX⁴, DTX⁴, DPX⁴, DGX⁴, NAX⁴, NSX⁴, NTX⁴, NPX⁴, NGX⁴, EAX⁴, ESX⁴, ETX⁴. EPX⁴, QAX⁴, QGX⁴, QAX⁴, QSX⁴, QTX⁴, QPX⁴, QGX⁴, HAX⁴, HSX⁴, HTX⁴, HPX⁴, HGX⁴, RAX⁴. RSX⁴, RTX⁴, RPX⁴, RGX⁴, LAX⁴, LSX⁴, LTX⁴, LPX⁴, LGX⁴, DADA (SEQ ID NO: 11), EAEA (SEQ ID NO: 12), DAEA (SEQ ID NO: 13), EADA (SEQ ID NO: 14), NANA (SEQ ID NO: 15), QAQA (SEQ ID NO: 16), DANA (SEQ ID NO: 17), EANA (SEQ ID NO: 18), NADA (SEQ ID NO: 19), NAQA (SEQ ID NO: 20), HAHA (SEQ ID NO: 21). RARA (SEQ ID NO: 22), LALA (SEQ ID NO: 23), HSHS (SEQ ID NO: 24), HTHT (SEQ ID NO: 25), LSLS (SEQ ID NO: 26), LTLT (SEQ ID NO: 27), RSRS (SEQ ID NO: 28), RTRT (SEQ ID NO: 29), HPHP (SEQ ID NO: 30), HGHG (SEQ ID NO: 31), LPLP (SEQ ID NO: 32), LGLG (SEQ ID NO: 33), DSDS (SEQ ID NO: 34), ESES (SEQ ID NO: 35), DSES (SEQ ID NO: 36), ESDS (SEQ ID NO: 37), NSNS (SEQ ID NO: 38), QSQS (SEQ ID NO: 39), DSNS (SEQ ID NO: 40), ESNS (SEQ ID NO: 41), NSDS (SEQ ID NO: 42), NSQS (SEQ ID NO: 43), DTDT (SEQ ID NO: 44), ETET (SEQ ID NO: 45), DTET (SEQ ID NO: 46), ETDT (SEQ ID NO: 47), NTNT (SEQ ID NO: 48), QTQT (SEQ ID NO: 49), DTNT (SEQ ID NO: 50), ETNT (SEQ ID NO: 51), NTDT (SEQ ID NO: 52), NTQT (SEQ ID NO: 53), DPDP (SEQ ID NO: 54), EPEP (SEQ ID NO: 55), DPEP (SEQ ID NO: 56), EPDP (SEQ ID NO: 57), NPNP (SEQ ID NO: 58), QPQP (SEQ ID NO: 59), DPNP (SEQ ID NO: 60), EPNP (SEQ ID NO: 61), NPDP (SEQ ID NO: 62), NPQP (SEQ ID NO: 63), DGDG (SEQ ID NO: 64), EGEG (SEQ ID NO: 65), DGEG (SEQ ID NO: 66), EGDG (SEQ ID NO: 67), NGNG (SEQ ID NO: 68), QGQG (SEQ ID NO: 69), DGNG (SEQ ID NO: 70), EGNG (SEQ ID NO: 71), NGDG (SEQ ID NO: 72), NGQG (SEQ ID NO: 73), HAHS (SEQ ID NO: 74), RARS (SEQ ID NO: 75), LALS (SEQ ID NO: 76), HSHT (SEQ ID NO: 77), HTHP (SEQ ID NO: 78), LSLT (SEQ ID NO: 79), LTLP (SEQ ID NO: 80), RSRT (SEQ ID NO: 81), RTRP (SEQ ID NO: 82), HPHG (SEQ ID NO: 83), HGHA (SEQ ID NO: 84), LPLA (SEQ ID NO: 85), LGLA (SEQ ID NO: 86), and combinations thereof, wherein X⁴ is any amino acid.

In one aspect of the presently disclosed subject matter, there is provided a method for preventing or treating a coronavirus infection in a subject in need thereof. In some embodiments, the method comprises: administering a vaccine composition as disclosed herein to the subject in an amount and via a route sufficient to induce an anti-coronavirus immune response in the subject.

In one aspect of the presently disclosed subject matter, there is provided a method for treating a viral infection, optionally a coronavirus infection or an HIV infection, further optionally a SARS-CoV infection, a SARS-CoV-2 infection, and/or a porcine epidemic diarrhea virus (PEDV) infection, in a subject in need thereof. In some embodiments, the method comprises administering to the subject a vaccine composition as disclosed herein in an amount and via a route sufficient to induce an anti-viral immune response in the subject.

In some embodiments, the vaccine composition is administered orally, rectally, vaginally, intra-nasally, parenterally, intradermally, subcutaneously, or intramuscularly. In some embodiments, the vaccine composition further comprises a pharmaceutically acceptable carrier, excipient, and/or diluent, optionally wherein the pharmaceutically acceptable carrier, excipient, and/or diluent is pharmaceutically acceptable for use in a human.

In some embodiments, a vaccine composition comprises and/or encodes (i) a coronavirus fusion peptide (FP) antigen, optionally a coronavirus FP antigen comprising an amino acid sequence selected from the group consisting of PSKPSKRSFIEDLLFNKVTLADAGF (SEQ ID NO: 2), PSKPSKRSFIEDLLFNKVTLADAG (SEQ ID NO: 1), SFIEDLLF (SEQ ID NO: 4), SFIEDLLFNKVTLADAGF (SEQ ID NO: 3), GRVVQKRSFIEDLLFNKVVTNGLG (SEQ ID NO: 5), and immunogenic fragments thereof; or (ii) an HIV antigen comprising an amino acid sequence selected from the group consisting of AVGIGAVF (SEQ ID NO: 7), ALGIGAAF (SEQ ID NO: 8), AVGFGAAF (SEQ ID NO: 9), AAGFGAMF (SEQ ID NO: 10), and immunogenic fragments thereof; or (iii) any combination thereof.

In one aspect of the presently disclosed subject matter, there is provided a composition comprising a modified bacterium or derivative thereof that expresses one or more tandem copies of a polypeptide of interest fused to a bacterial autotransporter β-barrel polypeptide, wherein (i) at least one copy of the one or more tandem copies of the polypeptide of interest is modified at or near its N-terminus to comprise an expression peptide comprising, consisting essentially of, or consisting of one or more copies of the amino acid sequence (X¹X²X³)ₙ, wherein each X¹ is independently selected from the group consisting of Asp (D), Asn (N), Glu (E), Gln (Q), His (H), Arg (R), and Lys (K); each X² is independently selected from the group consisting of Ala (A), Ser(S), Thr (T), Pro (P), Gly (G), Met (M), Leu (L), Ile (I), Val (V), and Cys (C); each X³ is absent in any given member of the one or more copies of the amino acid sequence (X¹X²X³)ₙ or, if present, is independently any amino acid; and n is at least 1; and (ii) the polypeptide of interest is expressed on a surface of the modified bacterium or the derivative thereof.

In one aspect of the presently disclosed subject matter, there is provided a composition for use in treating a patient infected with a virus, optionally a coronavirus, further optionally a SARS-CoV, SARS-CoV-2, and/or porcine epidemic diarrhea virus (PEDV), wherein the composition comprises a bacterium or derivative thereof, optionally a genome reduced derivative thereof, that expresses the expression cassette as disclosed herein on its surface, wherein polypeptide of interest in the expression cassette comprises a viral antigen modified to comprise an expression enhancement peptide. In some embodiments of the composition for use, the viral antigen is a coronavirus antigen or an HIV antigen, optionally a SARS-CoV, SARS-CoV-2, and/or porcine epidemic diarrhea virus (PEDV) antigen, further optionally wherein the viral antigen comprises (i) a coronavirus fusion peptide (FP) antigen, optionally a coronavirus FP antigen comprising an amino acid sequence selected from the group consisting of PSKPSKRSFIEDLLFNKVTLADAGF (SEQ ID NO: 2). PSKPSKRSFIEDLLFNKVTLADAG (SEQ ID NO: 1), SFIEDLLF (SEQ ID NO: 4). SFIEDLLFNKVTLADAGF (SEQ ID NO: 3), GRVVQKRSFIEDLLFNKVVTNGLG (SEQ ID NO: 5), and immunogenic fragments thereof; or (ii) an HIV antigen comprising an amino acid sequence selected from the group consisting of AVGIGAVF (SEQ ID NO: 7), ALGIGAAF (SEQ ID NO: 8). AVGFGAAF (SEQ ID NO: 9), AAGFGAMF (SEQ ID NO: 10), and immunogenic fragments thereof; or (iii) any combination thereof.

In one aspect of the presently disclosed subject matter, there is provided a composition for use in treating a subject infected with a virus, optionally a coronavirus or an HIV virus, further optionally a SARS-CoV virus, a SARS-CoV-2 virus, and/or porcine epidemic diarrhea virus (PEDV), wherein the composition comprises a bacterium or a genome reduced derivative thereof that expresses a protein comprising a viral antigen on its surface, wherein the protein comprises one or more tandem copies of the viral antigen at least one of which has been modified to comprise an expression enhancement peptide at or near its N-terminus. In some embodiments of the composition of use, the expression enhancement peptide comprises, consists essentially of, or consists of one or more copies of the amino acid sequence (X¹X²X³)ₙ, wherein each X¹ is independently selected from the group consisting of Asp (D), Asn (N), Glu (E), Gln (Q), His (H), Arg (R), and Lys (K); each X² is independently selected from the group consisting of Ala (A), Ser(S), Thr (T), Pro (P), Gly (G), Met (M), Leu (L), Ile (I), Val (V), and Cys (C); each X³ is absent in any given member of the one or more copies of the amino acid sequence (X¹X²X³)ₙ, or, if present, is independently any amino acid; and n is at least 1.

In some embodiments of the composition for use: the expression enhancement peptide comprises an amino acid sequence selected from the group consisting of DA, DS, DT, DP, DG, NA, NS, NT, NP, NG, EA, ES, ET, EP, QA, QG, QA, QS, QT, QP, QG, HA, HS, HT, HP, HG, RA, RS, RT, RP, RG, LA, LS, LT, LP, LG, DAX⁴, DSX⁴, DTX⁴, DPX⁴, DGX⁴, NAX⁴, NSX⁴, NTX⁴, NPX⁴, NGX⁴, EAX⁴, ESX⁴, ETX⁴, EPX⁴, QAX⁴, QGX⁴, QAX⁴, QSX⁴, QTX⁴, QPX⁴, QGX⁴, HAX⁴, HSX⁴, HTX⁴, HPX⁴, HGX⁴, RAX⁴, RSX⁴, RTX⁴, RPX⁴, RGX⁴, LAX⁴, LSX⁴, LTX⁴, LPX⁴, LGX⁴, DADA (SEQ ID NO: 11), EAEA (SEQ ID NO: 12), DAEA (SEQ ID NO: 13), EADA (SEQ ID NO: 14), NANA (SEQ ID NO: 15), QAQA (SEQ ID NO: 16), DANA (SEQ ID NO: 17), EANA (SEQ ID NO: 18), NADA (SEQ ID NO: 19), NAQA (SEQ ID NO: 20), HAHA (SEQ ID NO: 21). RARA (SEQ ID NO: 22), LALA (SEQ ID NO: 23), HSHS (SEQ ID NO: 24), HTHT (SEQ ID NO: 25), LSLS (SEQ ID NO: 26), LTLT (SEQ ID NO: 27), RSRS (SEQ ID NO: 28), RTRT (SEQ ID NO: 29), HPHP (SEQ ID NO: 30), HGHG (SEQ ID NO: 31), LPLP (SEQ ID NO: 32), LGLG (SEQ ID NO: 33), DSDS (SEQ ID NO: 34), ESES (SEQ ID NO: 35), DSES (SEQ ID NO: 36), ESDS (SEQ ID NO: 37), NSNS (SEQ ID NO: 38), QSQS (SEQ ID NO: 39), DSNS (SEQ ID NO: 40), ESNS (SEQ ID NO: 41), NSDS (SEQ ID NO: 42), NSQS (SEQ ID NO: 43), DTDT (SEQ ID NO: 44), ETET (SEQ ID NO: 45), DTET (SEQ ID NO: 46), ETDT (SEQ ID NO: 47), NTNT (SEQ ID NO: 48), QTQT (SEQ ID NO: 49), DTNT (SEQ ID NO: 50), ETNT (SEQ ID NO: 51), NTDT (SEQ ID NO: 52), NTQT (SEQ ID NO: 53), DPDP (SEQ ID NO: 54), EPEP (SEQ ID NO: 55), DPEP (SEQ ID NO: 56), EPDP (SEQ ID NO: 57), NPNP (SEQ ID NO: 58), QPQP (SEQ ID NO: 59), DPNP (SEQ ID NO: 60), EPNP (SEQ ID NO: 61). NPDP (SEQ ID NO: 62), NPQP (SEQ ID NO: 63), DGDG (SEQ ID NO: 64), EGEG (SEQ ID NO: 65), DGEG (SEQ ID NO: 66), EGDG (SEQ ID NO: 67), NGNG (SEQ ID NO: 68), QGQG (SEQ ID NO: 69), DGNG (SEQ ID NO: 70), EGNG (SEQ ID NO: 71), NGDG (SEQ ID NO: 72), NGQG (SEQ ID NO: 73), HAHS (SEQ ID NO: 74), RARS (SEQ ID NO: 75), LALS (SEQ ID NO: 76). HSHT (SEQ ID NO: 77), HTHP (SEQ ID NO: 78), LSLT (SEQ ID NO: 79), LTLP (SEQ ID NO: 80), RSRT (SEQ ID NO: 81), RTRP (SEQ ID NO: 82), HPHG (SEQ ID NO: 83), HGHA (SEQ ID NO: 84), LPLA (SEQ ID NO: 85), LGLA (SEQ ID NO: 86), and combinations thereof, wherein X⁴ is any amino acid.

In some embodiments of the composition for use, the viral antigen comprises a coronavirus antigen, optionally wherein the viral antigen comprises a SARS-CoV antigen, a SARS-CoV-2 antigen, or a porcine epidemic diarrhea virus (PEDV) antigen; or an HIV virus. In some embodiments of the composition of use, the viral antigen comprises (i) a coronavirus fusion peptide (FP) antigen, optionally a coronavirus FP antigen comprising an amino acid sequence selected from group the consisting of PSKPSKRSFIEDLLFNKVTLADAGF (SEQ ID NO: 2). PSKPSKRSFIEDLLFNKVTLADAG (SEQ ID NO: 1), SFIEDLLF (SEQ ID NO: 4). SFIEDLLFNKVTLADAGF (SEQ ID NO: 3), GRVVQKRSFIEDLLFNKVVINGLG (SEQ ID NO: 5), and immunogenic fragments thereof; or (ii) an HIV antigen comprising an amino acid sequence selected from the group consisting of AVGIGAVF (SEQ ID NO: 7), ALGIGAAF (SEQ ID NO: 8), AVGFGAAF (SEQ ID NO: 9), AAGFGAMF (SEQ ID NO: 10), and immunogenic fragments thereof; or (iii) any combination thereof.

In some embodiments of the composition for use, the genome reduced derivative thereof is a genome reduced derivative of a Gram negative bacterium. In some embodiments of the composition of use, the genome reduced derivative is an Escherichia coli. In some embodiments of the composition of use, the genome reduced derivative is a Salmonella bacterium. In some embodiments of the composition of use, the genome reduced derivative is the genome reduced derivative is a genome reduced E. coli.

In some embodiments of the composition for use, the genome reduced derivative is characterized by a reduced number of expressed genes of at least about 1.0%, 2.0%, 3.0%, 4.0%, 5.0%, 6.0%, 7.0%, 8.0%, 9.0%, 10%, 11%, 12%, 13%, 14%, 15%, or greater than 15% of genes relative to a non-genome reduced bacterium upon which the genome reduced bacterium or derivative thereof is based. In some embodiments of the composition of use, the reduced number of expressed genes comprises a reduction of expressed genes selected from the group consisting of at least about 2.4%, at least about 15.9%, and at least about 29.7% relative to a non-genome reduced bacterium upon which the genome reduced derivative thereof is based. In some embodiments of the composition of use the composition for use further comprises a pharmaceutically acceptable carrier, excipient, and/or diluent, optionally wherein the pharmaceutically acceptable carrier, excipient, and/or diluent is pharmaceutically acceptable for use in a human.

In one aspect of the presently disclosed subject matter, there is provided use of a composition comprising a bacterium or a genome reduced derivative thereof that expresses one or more tandem copies of a polypeptide of interest fused to a bacterial autotransporter β-barrel polypeptide for treating a subject infected with a virus, optionally a coronavirus, further optionally a SARS-CoV virus, a SARS-CoV-2 virus, and/or a porcine epidemic diarrhea virus (PEDV), wherein (i) at least one copy of the one or more tandem copies of the polypeptide of interest is modified at or near its N-terminus, to comprise an expression peptide comprising, consisting essentially of, or consisting of one or more copies of the amino acid sequence (X¹X²X³)ₙ, wherein each X¹ is independently selected from the group consisting of Asp (D), Asn (N), Glu (E), Gln (Q), His (H), Arg (R), and Lys (K); each X² is independently selected from the group consisting of Ala (A), Ser(S), Thr (T), Pro (P), Gly (G), Met (M), Leu (L), Ile (I), Val (V), and Cys (C); each X³ is absent in any given member of the one or more copies of the amino acid sequence (X¹X²X³)ₙ or, if present, is independently any amino acid; and n is at least 1; and (ii) the polypeptide of interest is expressed on a surface of the modified bacterium or the derivative thereof.

In some embodiments, a pharmaceutical composition comprising one or more components of the presently disclosed subject matter is administered orally. In one aspect, it is administered intra-nasally, rectally, vaginally, parenterally, employing intradermal, subcutaneously, or intramuscularly. In some embodiments, the pharmaceutical composition is a vaccine.

The system can also be used to express other viral proteins on the surface of bacteria to be used for immunization or treatment directed against the other viral proteins.

The presently disclosed subject matter provides a series of proteins or peptides and systems to produce or express those peptides in the context of cell structures, such as a lipid bilayer and other membrane structures found to have immunogenic activity that can be used singly or in combination to elicit an immunogenic response and are useful for preventing and treating viral infections (such as infections with coronaviruses, including but not limited to SARS-CoV and/or SARS-CoV-2 and/or PEDV. e.g. SARS-CoV-2 FP and/or PEDV FP). The presently disclosed subject matter could also be used to produce immunizing antigens targeting the conserved regions of other coronavirus virion envelope proteins for, for example, a universal coronavirus vaccine.

In some embodiments, the presently disclosed subject matter provides a modified bacterium expressing a set of peptides that can be used together as a cocktail or individually as a component of a vaccine (immunogen) to prevent or to treat any condition, disease, and/or disorder as described herein. When administered, the bacterium comprising the cocktail or combination of peptides elicits an immunogenic response. The presently disclosed subject matter further encompasses the use of biologically active homologues of the peptides and wells as biologically active fragments of the peptides. The homologues can, for example, comprise one of more conservative amino acid substitutions, additions, or deletions. The bacterium can be a wild type or genome reduced bacterium.

In some embodiments, the presently disclosed subject matter provides an immunogenic vaccine composition for use in treating and preventing infections, such as but not limited to coronavirus infections including but not limited to SARS-CoV and/or SARS-CoV-2 infections and/or PEDV infections. In some embodiments, the composition comprises at least one isolated peptide selected from the group of peptides disclosed herein, or biologically active fragments or homologs thereof. In some embodiments, the immunogenic vaccine composition is a system comprising a viral peptide provided by a bacterium in accordance with the presently disclosed subject matter. The vaccine composition can also include an adjuvant or a pharmaceutically acceptable carrier. In one aspect, at least two peptides are included in the composition. Any combination of the peptides can be used.

In some embodiments, an immunogenic fragment or homolog of a peptide of the presently disclosed subject matter is used. In some embodiments, the biologically active fragments or homologs of the peptide share at least about 50% sequence identity with the peptide. In some embodiments, they share at least about 75% sequence identity with the peptide. In yet other aspects, they share at least about 95% sequence identity with the peptide. Exemplary peptides that can be employed include peptides that can be modified and still give rise to an anti-coronavirus (e.g., anti-SARS-CoV and/or anti-SARS-CoV-2 and/or PEDV) immune response, and such sequences are also encompassed within the presently disclosed subject matter. Exemplary peptides that can be employed include peptides that comprise, consist essentially of, or consist of and/or are encoded by any of the sequences of the EXAMPLES. It is noted that the sequences represented by the EXAMPLES can be modified and still give rise to an anti-coronavirus (e.g., anti-SARS-CoV and/or anti-SARS-CoV-2) immune response, and such sequences are also encompassed within the presently disclosed subject matter.

In some embodiments, at least one of the active fragments or homologs being used comprises at least one conservative amino acid substitution. The presently disclosed subject matter encompasses the use of amino acid substitutions at any of the positions, as long as the resulting peptide maintains the desired biologic activity of being immunogenic. The presently disclosed subject matter further includes the peptides where amino acids have been deleted or inserted, as long as the resulting peptide maintains the desired biologic activity of being immunogenic.

In some embodiments, the methods of the presently disclosed subject matter provide for administering the vaccine composition to a subject at least about 2 times to about 50 times. In some embodiments, the method comprises administering the vaccine composition to a subject at least about 5 times to about 30 times. In some embodiments, the methods of the presently disclosed subject matter provide for administering the vaccine composition to a subject at least about 10 times to about 20 times. The method also provides for administering the composition daily, or weekly, or monthly. One of ordinary skill in the art can design a regimen based on the needs of a subject, taking into account the age, sex, and health of the subject.

As described herein, the peptides provided by the modified bacterium are immunogenic, so a useful composition comprising one or more of the peptides of the presently disclosed subject matter, even when using active fragments or homologs, or additionally short peptides, elicits an immunogenic response.

In some embodiments, a homolog of a peptide of the presently disclosed subject matter is one with one or more amino acid substitutions, deletions, or additions, and with the sequence identities described herein. In some embodiments, the substitution, deletion, or addition is conservative. In some embodiments, a serine or an alanine is substituted for a cysteine residue in a peptide of the presently disclosed subject matter.

In some embodiments, the subject is a mammal. In another embodiment, the mammal is a human.

The presently disclosed subject matter encompasses the use of purified isolated, recombinant, and synthetic peptides.

The presently disclosed subject matter further provides methods for producing peptides which are not easily soluble in an aqueous solution, by immediately expressing the peptides on the surface of the bacteria.

The methods and compositions of the presently disclosed subject matter encompass multiple regimens and dosages for administering the peptides of the presently disclosed subject matter for use in preventing and treating diseases and disorders caused by infectious agents. For example, a subject can be administered a combination of peptides, such as a combination of peptides provided by a bacterium, or a combination of bacteria expressing different peptides, of the presently disclosed subject matter once or more than once. The frequency and number of doses can vary based on many parameters, including the age, sex, and health of the subject. In some embodiments, up to 50 doses are administered. In some embodiments, up to 40 doses are administered, and in another up to 30 doses are administered. In some embodiments, up to 20 doses are administered, and in another up to 10 doses are administered. In some embodiments. 5-10 doses are administered. In some embodiments, 5, 6, 7, 8, 9, or 10 doses can be administered.

In some embodiments, bacteria expressing a peptide or bacteria expressing two or more peptides are administered more than once daily, in another daily, in another on alternating days, in another weekly, and in another, monthly. Treatment periods may be for a few days, or about a week, or about several weeks, or for several months. Follow-up administration or boosters can be used as well and the timing of that can be varied.

The amount of bacteria expressing a peptide or derivative of the bacteria administered per dose can vary as well. For example, in some embodiments, the compositions and methods of the presently disclosed subject matter include a range of peptide amounts (for example as provided by bacteria expressing a peptide) between about 1 nanogram of each peptide per dose to about 10 milligrams of immunogen per dose. In some embodiments, the number of micrograms is the same for each peptide. In some embodiments, the number of micrograms is not the same for each peptide. In some embodiments, the range of amounts of each immunogen administered per dose is from about 1 nanogram to about 10 milligrams.

Subjects can be monitored before and after bacteria administration for antibody levels against the immunogens being administered (for example as provided by bacteria expressing a peptide) and by monitoring T cell responses, including CD4⁺ and CD8⁺. Methods for these tests are routinely used in the art and are either described herein or, for example, in publications cited herein.

Although a vaccine composition construct, bacteria, mixture of bacteria, derivatives thereof, or cocktail of peptides or a combination therefor is described herein, when more than one bacterial construct or peptide is administered, each different bacterial construct or peptide can be administered separately. When a vaccine composition is administered more than once to a subject, the dose of each bacterial construct or peptide may vary per administration.

To increase the immunological response, various adjuvants may be used depending on the host species, including but not limited to cholera toxin B subunit. Freund's (complete and incomplete), mineral gels such as aluminum hydroxide, surface active substances such as lysolecithin, pluronic polyols, polyanions, peptides, oil emulsions, keyhole limpet hemocyanin, dinitrophenol, and potentially useful human adjuvants such as cholera toxin B subunit, alum, saponins, nucleic acids. LPS, BCG (bacille Calmette-Guerin) and Corynebacterium parvum.

If peptides are to be placed on the bacteria following exogenous production and not by protein synthesis by the bacteria themselves, those peptides for use in the presently disclosed subject matter may be readily prepared by standard, well-established techniques, such as solid-phase peptide synthesis (SPPS) as described by Stewart et al., 1984 and as described by Bodanszky & Bodanszky, 1984. At the outset, a suitably protected amino acid residue is attached through its carboxyl group to a derivatized, insoluble polymeric support, such as cross-linked polystyrene or polyamide resin. “Suitably protected” refers to the presence of protecting groups on both the α-amino group of the amino acid, and on any side chain functional groups. Side chain protecting groups are generally stable to the solvents, reagents and reaction conditions used throughout the synthesis, and are removable under conditions which will not affect the final peptide product. Stepwise synthesis of the oligopeptide is carried out by the removal of the N-protecting group from the initial amino acid, and couple thereto of the carboxyl end of the next amino acid in the sequence of the desired peptide. This amino acid is also suitably protected. The carboxyl of the incoming amino acid can be activated to react with the N-terminus of the support-bound amino acid by formation into a reactive group such as formation into a carbodiimide, a symmetric acid anhydride or an “active ester” group such as hydroxy benzotriazole or pentafluorophenyl esters.

Examples of solid phase peptide synthesis methods include the BOC method which utilized tert-butyloxicarbonyl as the α-amino protecting group, and the FMOC method which utilizes 9-fluorenylmethyloxycarbonyl to protect the α-amino of the amino acid residues, both methods of which are well known by those of skill in the art.

Incorporation of N- and/or C-blocking groups can also be achieved using protocols conventional to solid phase peptide synthesis methods. For incorporation of C-terminal blocking groups, for example, synthesis of the desired peptide is typically performed using, as solid phase, a supporting resin that has been chemically modified so that cleavage from the resin results in a peptide having the desired C-terminal blocking group. To provide peptides in which the C-terminus bears a primary amino blocking group, for instance, synthesis is performed using a p-methylbenzhydrylamine (MBHA) resin so that, when peptide synthesis is completed, treatment with hydrofluoric acid releases the desired C-terminally amidated peptide. Similarly, incorporation of an N-methylamine blocking group at the C-terminus is achieved using N-methylaminoethyl-derivatized DVB, resin, which upon HF treatment releases a peptide bearing an N-methylamidated C-terminus. Blockage of the C-terminus by esterification can also be achieved using conventional procedures. This entails use of resin/blocking group combination that permits release of side-chain peptide from the resin, to allow for subsequent reaction with the desired alcohol, to form the ester function. FMOC protecting group, in combination with DVB resin derivatized with methoxyalkoxybenzyl alcohol or equivalent linker, can be used for this purpose, with cleavage from the support being effected by TFA in dichloromethane. Esterification of the suitably activated carboxyl function e.g., with DCC, can then proceed by addition of the desired alcohol, followed by deprotection and isolation of the esterified peptide product.

Incorporation of N-terminal blocking groups can be achieved while the synthesized peptide is still attached to the resin, for instance by treatment with a suitable anhydride and nitrile. To incorporate an acetyl-blocking group at the N-terminus, for instance, the resin-coupled peptide can be treated with 20% acetic anhydride in acetonitrile. The N-blocked peptide product can then be cleaved from the resin, deprotected and subsequently isolated.

To ensure that the peptide obtained from either chemical or biological synthetic techniques is the desired peptide, analysis of the peptide composition should be conducted. Such amino acid composition analysis may be conducted using high-resolution mass spectrometry to determine the molecular weight of the peptide. Alternatively, or additionally, the amino acid content of the peptide can be confirmed by hydrolyzing the peptide in aqueous acid, and separating, identifying, and quantifying the components of the mixture using HPLC, or an amino acid analyzer. Protein sequenators, which sequentially degrade the peptide and identify the amino acids in order, may also be used to determine definitely the sequence of the peptide. Prior to its use, the peptide is purified to remove contaminants. In this regard, it will be appreciated that the peptide will be purified so as to meet the standards set out by the appropriate regulatory agencies. Any one of a number of a conventional purification procedures may be used to attain the required level of purity including, for example, reversed-phase high-pressure liquid chromatography (HPLC) using an alkylated silica column such as C₄₋, C₈₋, or C₁₈₋ silica. A gradient mobile phase of increasing organic content is generally used to achieve purification, for example, acetonitrile in an aqueous buffer, usually containing a small amount of trifluoroacetic acid. Ion-exchange chromatography can be also used to separate peptides based on their charge.

It will be appreciated, of course, that the peptides or antibodies, derivatives, or fragments thereof may incorporate amino acid residues which are modified without affecting activity. For example, the termini may be derivatized to include blocking groups, i.e., chemical substituents suitable to protect and/or stabilize the N- and C-termini from “undesirable degradation”, a term meant to encompass any type of enzymatic, chemical, or biochemical breakdown of the compound at its termini which is likely to affect the function of the compound. i.e. sequential degradation of the compound at a terminal end thereof.

Blocking groups include protecting groups conventionally used in the art of peptide chemistry which will not adversely affect the in vivo activities of the peptide. For example, suitable N-terminal blocking groups can be introduced by alkylation or acylation of the N-terminus. Examples of suitable N-terminal blocking groups include C₁-C₅ branched or unbranched alkyl groups, acyl groups such as formyl and acetyl groups, as well as substituted forms thereof, such as the acetamidomethyl (Acm) group. Desamino analogs of amino acids are also useful N-terminal blocking groups, and can either be coupled to the N-terminus of the peptide or used in place of the N-terminal reside. Suitable C-terminal blocking groups, in which the carboxyl group of the C-terminus is either incorporated or not, include esters, ketones or amides. Ester or ketone-forming alkyl groups, particularly lower alkyl groups such as methyl, ethyl and propyl, and amide-forming amino groups such as primary amines (—NH₂), and mono- and di-alkylamino groups such as methylamino, ethylamino, dimethylamino, diethylamino, methylethylamino and the like are examples of C-terminal blocking groups. Decarboxylated amino acid analogues such as agmatine are also useful C-terminal blocking groups and can be either coupled to the peptide's C-terminal residue or used in place of it. Further, it will be appreciated that the free amino and carboxyl groups at the termini can be removed altogether from the peptide to yield desamino and decarboxylated forms thereof without affect on peptide activity.

Other modifications can also be incorporated without adversely affecting the activity and these include, but are not limited to, substitution of one or more of the amino acids in the natural L-isomeric form with amino acids in the D-isomeric form. Thus, the peptide may include one or more D-amino acid resides, or may comprise amino acids which are all in the D-form. Retro-inverso forms of peptides in accordance with the presently disclosed subject matter are also contemplated, for example, inverted peptides in which all amino acids are substituted with D-amino acid forms.

Acid addition salts of the presently disclosed subject matter are also contemplated as functional equivalents. Thus, a peptide in accordance with the presently disclosed subject matter treated with an inorganic acid such as hydrochloric, hydrobromic, sulfuric, nitric, phosphoric, and the like, or an organic acid such as an acetic, propionic, glycolic, pyruvic, oxalic, malic, malonic, succinic, maleic, fumaric, tartaric, citric, benzoic, cinnamic, mandelic, methanesulfonic, ethanesulfonic, p-toluenesulfonic, salicyclic and the like, to provide a water soluble salt of the peptide is suitable for use in the presently disclosed subject matter, for example a GR bacteria with attached additional immunogens.

The presently disclosed subject matter also provides for homologs of proteins and peptides for use in accordance with the presently disclosed subject matter. Homologs can differ from naturally occurring proteins or peptides by conservative amino acid sequence differences or by modifications which do not affect sequence, or by both.

For example, conservative amino acid changes may be made, which although they alter the primary sequence of the protein or peptide, do not normally alter its function. To that end. 10 or more conservative amino acid changes typically have no effect on protein function.

Modifications (which do not normally alter primary sequence) include in vivo, or in vitro chemical derivatization of polypeptides, e.g., acetylation, or carboxylation. Also included are modifications of glycosylation, e.g., those made by modifying the glycosylation patterns of a polypeptide during its synthesis and processing or in further processing steps: e.g., by exposing the polypeptide to enzymes which affect glycosylation, e.g., mammalian glycosylating or deglycosylating enzymes. Also embraced are sequences which have phosphorylated amino acid residues, e.g., phosphotyrosine, phosphoserine, or phosphothreonine.

Also included are polypeptides or antibody fragments which have been modified using ordinary molecular biological techniques so as to improve their resistance to proteolytic degradation or to optimize solubility properties or to render them more suitable as a therapeutic agent. Homologs of such polypeptides include those containing residues other than naturally occurring L-amino acids, e.g., D-amino acids or non-naturally occurring synthetic amino acids. The peptides of the presently disclosed subject matter are not limited to products of any of the specific exemplary processes listed herein.

Substantially pure protein or peptide obtained as described herein may be purified by following known procedures for protein purification, wherein an immunological, enzymatic, or other assay is used to monitor purification at each stage in the procedure. Protein purification methods are well known in the art, and are described, for example in Deutscher et al., 1990.

One of ordinary skill in the art will appreciate that when more than one peptide is used (for example as provided by a bacterium expressing two or more peptides or by different bacteria expressing different peptides or derivative of the bacterium) that they do not necessarily have to be administered in the same pharmaceutical composition at the same time, and that multiple administrations can also be used. When multiple injections are used, they can be administered, for example, in a short sequence such as one right after the other or they can be spaced out over predetermined periods of time, such as every 5 minutes, every 10 minutes, every 30 minutes, etc. Of course, administration can also be performed by administering a pharmaceutical comprising all components to be administered, such as a cocktail comprising bacteria expressing a peptide or derivative of the bacteria of the presently disclosed subject matter. It can also be appreciated that a treatment regimen may include more than one round of injections, spaced over time such as weeks or months, and can be altered according to the effectiveness of the treatment on the particular subject being treated.

The presently disclosed subject matter provides multiple methods of using specifically prepared bacteria expressing a peptide or derivative of the bacteria, for example, in fresh or lyophilized liposome, proper routes of administration of the bacteria or derivative thereof, proper doses of the bacteria or derivative thereof, and specific combinations of heterologous immunization including priming in one administration route followed by liposome-mediated antigen boost in a different route to tailor the immune responses in respects of enhancing cell mediated immune response, cytokine secretion, humoral immune response, especially skewing T helper responses to be Th1 or a balanced Th1 and Th2 type. For more detail, see U.S. patent application Ser. No. 11/572,453 (published as U.S. Patent Application Publication No. 2008/0193469, which is now U.S. Pat. No. 8,012,932 and each of which is incorporated herein by reference in its entirety), which claims priority to PCT International Patent Application Serial No. PCT/US2005/026102 (published as PCT International Patent Application Publication No. WO 2006/012539 and incorporated herein by reference in its entirety).

A homolog herein is understood to comprise an immunogenic peptide having in some embodiments at least 70%, in some embodiments at least 80%, in some embodiments at least 90%, in some embodiments at least 95%, in some embodiments at least 98%, and in some embodiments at least 99% amino acid sequence identity with the peptides mentioned above and is still capable of eliciting at least the immune response obtainable thereby. A homolog or analog may herein comprise substitutions, insertions, deletions, additional N- or C-terminal amino acids, and/or additional chemical moieties, such as carbohydrates, to increase stability, solubility, and immunogenicity.

In one embodiment of the presently disclosed subject matter, the present immunogenic polypeptides as defined herein, are glycosylated. Without wishing to be bound by any particular theory, it is hypothesized herein that by glycosylation of these polypeptides the immunogenicity thereof may be increased. Therefore, in one embodiment, the aforementioned immunogenic polypeptide as defined herein before, is glycosylated, having a carbohydrate content varying from 10-80 wt %, based on the total weight of the glycoprotein or glycosylated polypeptide. Said carbohydrate content ranges can be from 15-70 wt %, or from 20-60 wt %. In another embodiment, said glycosylated immunogenic polypeptide comprises a glycosylation pattern that is similar to that of the peptides of the human that is treated. It is hypothesized that this even further increases the immunogenicity of said polypeptide. Thus, in one embodiment, the immunogenic polypeptide comprises a glycosylation pattern that is similar to that of the corresponding glycoprotein. In one embodiment, the source of a peptide comprises an effective amount of at least one immunogenic peptide selected from the peptides described herein, and immunologically active homologs thereof and fragments thereof, or a nucleic acid sequence encoding said immunogenic peptide.

In one embodiment, the present method of immunization comprises the administration of a source of immunogenically active peptide fragments, said peptide fragments being selected from the peptide fragments and/or homologs thereof as defined herein before.

Peptides may advantageously be chemically synthesized and may optionally be (partially) overlapping and/or may also be ligated to other molecules, peptides, or proteins. Peptides may also be fused to form synthetic proteins, as in Welters et al., 2004. It may also be advantageous to add to the amino- or carboxy-terminus of the peptide chemical moieties or additional (modified or D-) amino acids in order to increase the stability and/or decrease the biodegradability of the peptide. To improve immunogenicity, immuno-stimulating moieties may be attached, e.g., by lipidation or glycosylation. To enhance the solubility of the peptide, addition of charged or polar amino acids may be used, in order to enhance solubility and increase stability in vivo.

For immunization purposes, the aforementioned immunogenic peptides for use with the presently disclosed subject matter may also be fused with proteins, such as, but not limited to, tetanus toxin/toxoid, diphtheria toxin/toxoid or other carrier molecules. The polypeptides according to the presently disclosed subject matter may also be advantageously fused to heat shock proteins, such as recombinant endogenous (murine) gp96 (GRP94) as a carrier for immunodominant peptides as described in (see e.g., Rapp & Kaufmann. 2004; Zugel, 2001), or fusion proteins with Hsp70 (PCT International Patent Application Publication No. WO 1999/54464).

The individual amino acid residues of the present immunogenic (poly) peptides for use with the presently disclosed subject matter can be incorporated in the peptide by a peptide bond or peptide bond mimetic. A peptide bond mimetic of the presently disclosed subject matter includes peptide backbone modifications well known to those skilled in the art. Such modifications include modifications of the amide nitrogen, the alpha carbon, amide carbonyl, complete replacement of the amide bond, extensions, deletions, or backbone cross-links. See generally, Spatola, 1983. Several peptide backbone modifications are known and can be used in the practice of the presently disclosed subject matter.

Amino acid mimetics may also be incorporated in the polypeptides. An “amino acid mimetic” as used here is a moiety other than a naturally occurring amino acid that conformationally and functionally serves as a substitute for an amino acid in a polypeptide of the presently disclosed subject matter. Such a moiety serves as a substitute for an amino acid residue if it does not interfere with the ability of the peptide to elicit an immune response, Amino acid mimetics may include non-protein amino acids. A number of suitable amino acid mimetics are known to the skilled artisan, they include cyclohexylalanine, 3-cyclohexylpropionic acid. L-adamantyl alanine, adamantylacetyl acid and the like. Peptide mimetics suitable for peptides of the presently disclosed subject matter are discussed by Morgan & Gainor, 1989.

In some embodiments, the present method comprises the administration of a composition (e.g., bacteria or derivative thereof) comprising one or more of the present immunogenic peptides as defined herein above, and at least one excipient. Excipients are well known in the art of pharmacy and may for instance be found in textbooks such as Remington's Pharmaceutical Sciences, 18th ed. (1990).

The present method for immunization may further comprise the administration, and in one aspect, the co-administration of at least one adjuvant. Adjuvants may comprise any adjuvant known in the art of vaccination or composition for eliciting an immune response and may be selected using textbooks like Colligan et al., 1994-2004.

Adjuvants are herein intended to include any substance or compound that, when used, in combination with an antigen, to immunize a human or an animal, stimulates the immune system, thereby provoking, enhancing, or facilitating the immune response against the antigen, preferably without generating a specific immune response to the adjuvant itself. In one aspect, adjuvants can enhance the immune response against a given antigen by at least a factor of 1.5, 2, 2.5, 5, 10, or 20, as compared to the immune response generated against the antigen under the same conditions but in the absence of the adjuvant. Tests for determining the statistical average enhancement of the immune response against a given antigen as produced by an adjuvant in a group of animals or humans over a corresponding control group are available in the art. The adjuvant preferably is capable of enhancing the immune response against at least two different antigens. The adjuvant of the presently disclosed subject matter will usually be a compound that is foreign to a human, thereby excluding immunostimulatory compounds that are endogenous to humans, such as e.g., interleukins, interferons, and other hormones.

A number of adjuvants are well known to one of ordinary skill in the art. Suitable adjuvants include, e.g., incomplete Freund's adjuvant, alum, aluminum phosphate, aluminum hydroxide. N-acetyl-muramyl-L-threonyl-D-isoglutamine (thr-MDP). N-acetyl-nor-muramyl-L-alanyl-D-isoglutamine (CGP 11637, referred to as nor-MDP). N-acetylmuramyl-L-alanyl-D-isoglutaminyl-L-alanine-2-(1′-2′-dipalmitoyl-sn-glycero-3-hydroxy-phosphoryloxy)-ethylamine (CGP 19835A, referred to as MTP-PE), DDA (2 dimethyldioctadecylammonium bromide), poly IC, Poly-A-poly-U, RIBI™, GERBU™, PAM3™, CARBOPOL™, SPECOL™, TITERMAX™, tetanus toxoid, diphtheria toxoid, meningococcal outer membrane proteins, cholera toxin B subunit, diphtheria protein CRM₁₉₇. Preferred adjuvants comprise a ligand that is recognized by a Toll-like-receptor (TLR) present on antigen presenting cells. Various ligands recognized by TLR's are known in the art and include e.g., lipopeptides (see e.g., PCT International Patent Application Publication No. WO 2004/110486), lipopolysaccharides, peptidoglycans, lipoteichoic acids, lipoarabinomannans, lipoproteins (from mycoplasma or spirochetes), double-stranded RNA (poly I:C), unmethylated DNA, flagellin. CpG-containing DNA, and imidazoquinolines, as well derivatives of these ligands having chemical modifications.

In some embodiments of the present methods, one or more bacteria expressing a peptide or derivative of the bacteria are typically administered at a dosage of about 1 μg/kg patient body weight or more at least once. Often dosages are greater than 10 μg/kg. According to the presently disclosed subject matter, the dosages range in some embodiments from 1 μg/kg to 1 mg/kg.

In some embodiments typical dosage regimens comprise administering a dosage of in some embodiments 1-1000 μg/kg, in some embodiments 10-500 μg/kg, in some embodiments 10-150 μg/kg, once, twice, or three times a week for a period of one, two, three, four or five weeks. According to some embodiments. 10-100 μg/kg is administered once a week for a period of one or two weeks.

The presently disclosed methods, in some embodiments, comprise administration of bacteria expressing a peptide or derivative of the bacteria and compositions comprising them via the injection, transdermal, intranasal, or oral route. In some embodiments of the presently disclosed subject matter, the present method comprises vaginal or rectal administration of the present bacteria expressing a peptide or derivative of the bacteria and compositions comprising them.

Another aspect of the presently disclosed subject matter relates to a pharmaceutical preparation comprising as the active ingredient the present source of a polypeptide as defined herein before. More particularly pharmaceutical preparation comprises as the active ingredient one or more of the aforementioned immunogenic peptides, homologs thereof and fragments of said peptides and homologs thereof, as provided by a bacteria expressing a peptide or derivative of the bacteria as defined herein above.

The presently disclosed subject matter further provides a pharmaceutical preparation comprising one or more bacteria expressing a peptide or derivative of the bacteria of the presently disclosed subject matter. The concentration of said peptides in the pharmaceutical composition can vary widely, i.e., from less than about 0.1% by weight, usually being at least about 1% by weight to as much as 20% by weight or more.

The composition may comprise a pharmaceutically acceptable carrier in addition to the active ingredient. The pharmaceutical carrier can be any compatible, non-toxic substance suitable to deliver the immunogenic peptide or bacteria expressing a peptide or derivative of the bacteria to the patient. For polypeptides, sterile water, alcohol, fats, waxes, and inert solids may be used as the carrier. Pharmaceutically acceptable adjuvants, buffering agents, dispersing agents, and the like, may also be incorporated into the pharmaceutical compositions.

In some embodiments, the present bacteria expressing a peptide or derivative of the bacteria are administered by injection. The parenteral route for administration is in accordance with known methods, e.g., injection or infusion by intravenous, intraperitoneal, intramuscular, intra-arterial, subcutaneous, rectal, vaginal, or intralesional routes. The bacteria expressing a peptide or derivative of the bacteria may be administered continuously by infusion or by bolus injection. In some embodiments, a composition for intravenous infusion could be made up to contain 10 to 50 ml of sterile 0.9% NaCl or 5% glucose optionally supplemented with a 20% albumin solution and in some embodiments between 10 μg and 50 mg, in some embodiments between 50 μg and 10 mg, of the bacteria expressing a peptide or derivative of the bacteria. A typical pharmaceutical composition for intramuscular injection would be made up to contain, for example 1-10 ml of sterile buffered water and in some embodiments between 10 μg and 50 mg, in some embodiments between 50 μg and 10 mg, of the bacteria expressing a peptide or derivative of the bacteria of the presently disclosed subject matter. Methods for preparing parenterally administrable compositions are well known in the art and described in more detail in various sources, including, for example Remington's Pharmaceutical Sciences, 18th ed . . . 1990, incorporated by reference in its entirety for all purposes).

For convenience, immune responses are often described in the presently disclosed subject matter as being either “primary” or “secondary” immune responses. A primary immune response, which is also described as a “protective” immune response, refers to an immune response produced in an individual as a result of some initial exposure (e.g., the initial “immunization”) to a particular antigen. Such an immunization can occur, for example, as the result of some natural exposure to the antigen (for example, from initial infection by some pathogen that exhibits or presents the antigen). Alternatively, the immunization can occur because of vaccinating the individual with a vaccine containing the antigen. For example, the vaccine can be a vaccine comprising one or more antigenic epitopes or fragments of the peptides of the presently disclosed subject matter.

In certain embodiments, the disclosed methods and compositions may involve preparing peptides with one or more substituted amino acid residues. In various embodiments, the structural, physical and/or therapeutic characteristics of peptide sequences may be optimized by replacing one or more amino acid residues.

In some embodiments, the presently disclosed subject matter encompasses the substitution of a serine or an alanine residue for a cysteine residue in a peptide of the presently disclosed subject matter. Support for this includes what is known in the art. For example, see the following citation for justification of such a serine or alanine substitution: Kittlesen et al., 1998.

Other modifications can also be incorporated without adversely affecting the activity and these include, but are not limited to, substitution of one or more of the amino acids in the natural L-isomeric form with amino acids in the D-isomeric form. Thus, the peptide may include one or more D-amino acid resides, or may comprise amino acids which are all in the D-form. Retro-inverso forms of peptides in accordance with the presently disclosed subject matter are also contemplated, for example, inverted peptides in which all amino acids are substituted with D-amino acid forms.

The skilled artisan will be aware that, in general, amino acid substitutions in a peptide typically involve the replacement of an amino acid with another amino acid of relatively similar properties (i.e., conservative amino acid substitutions). The properties of the various amino acids and effect of amino acid substitution on protein structure and function have been the subject of extensive study and knowledge in the art. For example, one can make the following isosteric and/or conservative amino acid changes in the parent polypeptide sequence with the expectation that the resulting polypeptides would have a similar or improved profile of the properties described above:

Substitution of alkyl-substituted hydrophobic amino acids: including alanine, leucine, isoleucine, valine, norleucine. S-2-aminobutyric acid. S-cyclohexylalanine or other simple alpha-amino acids substituted by an aliphatic side chain from C₁-C₁₀ carbons including branched, cyclic, and straight chain alkyl, alkenyl or alkynyl substitutions.

Substitution of aromatic-substituted hydrophobic amino acids: including phenylalanine, tryptophan, tyrosine, biphenylalanine, 1-naphthylalanine, 2-naphthylalanine, 2-benzothienylalanine, 3-benzothienylalanine, histidine, amino, alkylamino, dialkylamino, aza, halogenated (fluoro, chloro, bromo, or iodo) or alkoxy-substituted forms of the previous listed aromatic amino acids, illustrative examples of which are: 2-, 3- or 4-aminophenylalanine, 2-, 3- or 4-chlorophenylalanine, 2-, 3- or 4-methylphenylalanine, 2-, 3- or 4-methoxyphenylalanine, 5-amino-, 5-chloro-, 5-methyl- or 5-methoxytryptophan. 2′-, 3′-, or 4′-amino-, 2′-, 3′-, or 4′-chloro-, 2,3, or 4-biphenylalanine, 2′, −3′, —or 4′-methyl-2, 3 or 4-biphenylalanine, and 2- or 3-pyridylalanine.

Substitution of amino acids containing basic functions: including arginine, lysine, histidine, ornithine, 2,3-diaminopropionic acid, homoarginine, alkyl, alkenyl, or aryl-substituted (from C₁-C₁₀ branched, linear, or cyclic) derivatives of the previous amino acids, whether the substituent is on the heteroatoms (such as the alpha nitrogen, or the distal nitrogen or nitrogens, or on the alpha carbon, in the pro-R position for example. Compounds that serve as illustrative examples include: N-epsilon-isopropyl-lysine, 3-(4-tetrahydropyridyl)-glycine, 3-(4-tetrahydropyridyl)-alanine, N,N-gamma, gamma-diethyl-homoarginine. Included also are compounds such as alpha methyl arginine, alpha methyl 2,3-diaminopropionic acid, alpha methyl histidine, alpha methyl ornithine where alkyl group occupies the pro-R position of the alpha carbon. Also included are the amides formed from alkyl, aromatic, heteroaromatic (where the heteroaromatic group has one or more nitrogens, oxygens, or sulfur atoms singly or in combination) carboxylic acids or any of the many well-known activated derivatives such as acid chlorides, active esters, active azolides and related derivatives) and lysine, ornithine, or 2,3-diaminopropionic acid.

Substitution of acidic amino acids: including aspartic acid, glutamic acid, homoglutamic acid, tyrosine, alkyl, aryl, arylalkyl, and heteroaryl sulfonamides of 2,4-diaminopropionic acid, ornithine or lysine and tetrazole-substituted alkyl amino acids.

Substitution of side chain amide residues: including asparagine, glutamine, and alkyl or aromatic substituted derivatives of asparagine or glutamine.

Substitution of hydroxyl containing amino acids: including serine, threonine, homoserine. 2,3-diaminopropionic acid, and alkyl or aromatic substituted derivatives of serine or threonine. It is also understood that the amino acids within each of the categories listed above can be substituted for another of the same group.

For example, the hydropathic index of amino acids may be considered (Kyte & Doolittle. 1982). The relative hydropathic character of the amino acid contributes to the secondary structure of the resultant protein, which in turn defines the interaction of the protein with other molecules. Each amino acid has been assigned a hydropathic index on the basis of its hydrophobicity and charge characteristics (Kyte & Doolittle. 1982), these are: isoleucine (+4.5); valine (+4.2); leucine (+3.8); phenylalanine (+2.8); cysteine/cystine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5). In making conservative substitutions, the use of amino acids whose hydropathic indices are within +/−2 is preferred, within +/−1 are more preferred, and within +/−0.5 are even more preferred.

Amino acid substitution may also take into account the hydrophilicity of the amino acid residue (e.g., U.S. Pat. No. 4,554,101). Hydrophilicity values have been assigned to amino acid residues: arginine (+3.0); lysine (+3.0); aspartate (+3.0); glutamate (+3.0); serine (+0.3); asparagine (+0.2); glutamine (+0.2); glycine (0)); threonine (−0.4); proline (−0.5.+−0.1); alanine (−0.5); histidine (−0.5); cysteine (−1.0); methionine (−1.3); valine (−1.5); leucine (−1.8); isoleucine (−1.8); tyrosine (−2.3); phenylalanine (−2.5); tryptophan (−3.4). Replacement of amino acids with others of similar hydrophilicity is preferred.

Other considerations include the size of the amino acid side chain. For example, it would generally not be preferred to replace an amino acid with a compact side chain, such as glycine or serine, with an amino acid with a bulky side chain, e.g., tryptophan or tyrosine. The effect of various amino acid residues on protein secondary structure is also a consideration. Through empirical study, the effect of different amino acid residues on the tendency of protein domains to adopt an alpha-helical, beta-sheet or reverse turn secondary structure has been determined and is known in the art (see e.g., Chou & Fasman, 1974; Chou & Fasman. 1978; Chou & Fasman, 1979).

Based on such considerations and extensive empirical study, tables of conservative amino acid substitutions have been constructed and are known in the art. For example: arginine and lysine; glutamate and aspartate; serine and threonine; glutamine and asparagine; and valine, leucine and isoleucine. Alternatively: Ala (A) Leu, Ile, Val; Arg (R) Gln, Asn, Lys; Asn (N) His, Asp, Lys, Arg, Gln; Asp (D) Asn, Glu; Cys (C) Ala, Ser; Gln (Q) Glu, Asn; Glu (E) Gln, Asp; Gly (G) Ala; His (H) Asn, Gln, Lys, Arg; Ile (I) Val, Met, Ala, Phe, Leu; Leu (L) Val, Met, Ala, Phe, Ile; Lys (K) Gln, Asn, Arg; Met (M) Phe, Ile, Leu; Phe (F) Leu, Val, Ile, Ala, Tyr; Pro (P) Ala; Ser(S), Thr; Thr (T) Ser; Trp (W) Phe, Tyr; Tyr (Y) Trp, Phe, Thr, Ser; Val (V) Ile, Leu, Met, Phe, Ala.

Other considerations for amino acid substitutions include whether or not the residue is located in the interior of a protein or is solvent exposed. For interior residues, conservative substitutions would include: Asp and Asn; Ser and Thr; Ser and Ala; Thr and Ala; Ala and Gly; Ile and Val; Val and Leu; Leu and Ile; Leu and Met; Phe and Tyr; Tyr and Trp (see e.g., the PROWL Rockefeller University website). For solvent exposed residues, conservative substitutions would include: Asp and Asn; Asp and Glu; Glu and Gln; Glu and Ala; Gly and Asn; Ala and Pro; Ala and Gly; Ala and Ser; Ala and Lys; Ser and Thr; Lys and Arg; Val and Leu; Leu and Ile; Ile and Val; Phe and Tyr (see e.g., the PROWL Rockefeller University website). Various matrices have been constructed to assist in selection of amino acid substitutions, such as the PAM250 scoring matrix. Dayhoff matrix, Grantham matrix. McLachlan matrix. Doolittle matrix. Henikoff matrix. Miyata matrix. Fitch matrix. Jones matrix. Rao matrix. Levin matrix and Risler matrix (see e.g., the PROWL Rockefeller University website).

In determining amino acid substitutions, one may also consider the existence of intermolecular or intramolecular bonds, such as formation of ionic bonds (salt bridges) between positively charged residues (e.g., His. Arg. Lys) and negatively charged residues (e.g., Asp. Glu) or disulfide bonds between nearby cysteine residues.

Methods of substituting any amino acid for any other amino acid in an encoded peptide sequence are well known and a matter of routine experimentation for the skilled artisan, for example by the technique of site-directed mutagenesis or by synthesis and assembly of oligonucleotides encoding an amino acid substitution and splicing into an expression vector construct.

The presently disclosed subject matter is also directed to methods of administering the compounds of the presently disclosed subject matter to a subject.

Pharmaceutical compositions comprising the present compositions are administered to an individual in need thereof by any number of routes including, but not limited to, topical, oral, rectally, vaginally, intravenous, intramuscular, intra-arterial, intramedullary, intrathecal, intraventricular, transdermal, subcutaneous, intraperitoneal, intranasal, enteral, topical, sublingual, or rectal means.

The presently disclosed subject matter is also directed to pharmaceutical compositions comprising the bacteria of the presently disclosed subject matter. More particularly, such compounds can be formulated as pharmaceutical compositions using standard pharmaceutically acceptable carriers, fillers, solubilizing agents and stabilizers known to those skilled in the art.

The presently disclosed subject matter also encompasses the use pharmaceutical compositions of an appropriate compound, homolog, fragment, analog, or derivative thereof to practice the methods of the presently disclosed subject matter, the composition comprising at least one appropriate compound, homolog, fragment, analog, or derivative thereof and a pharmaceutically acceptable carrier.

The pharmaceutical compositions useful for practicing the presently disclosed subject matter may be administered to deliver a dose of between 1 ng/kg/day and 100 mg/kg/day. Pharmaceutical compositions that are useful in the methods of the presently disclosed subject matter may be administered systemically in oral solid formulations, ophthalmic, suppository, aerosol, topical or other similar formulations. In addition to the appropriate compound, such pharmaceutical compositions may contain pharmaceutically acceptable carriers and other ingredients known to enhance and facilitate drug administration. Other possible formulations, such as nanoparticles, liposomes, resealed erythrocytes, and immunologically based systems may also be used to administer an appropriate compound according to the methods of the presently disclosed subject matter.

As used herein, the term “physiologically acceptable” ester or salt means an ester or salt form of the active ingredient which is compatible with any other ingredients of the pharmaceutical composition, which is not deleterious to the subject to which the composition is to be administered.

The formulations of the pharmaceutical compositions described herein may be prepared by any method known or hereafter developed in the art of pharmacology. In general, such preparatory methods include the step of bringing the active ingredient into association with a carrier or one or more other accessory ingredients, and then, if necessary or desirable, shaping or packaging the product into a desired single- or multi-dose unit.

Although the descriptions of pharmaceutical compositions provided herein are principally directed to pharmaceutical compositions which are suitable for ethical administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and perform such modification with merely ordinary, if any, experimentation.

Subjects to which administration of the pharmaceutical compositions of the presently disclosed subject matter is contemplated include, but are not limited to, humans and other primates, mammals including commercially relevant mammals such as cattle, pigs, horses, sheep, cats, and dogs, birds including commercially relevant birds such as chickens, ducks, geese, and turkeys.

Pharmaceutical compositions that are useful in the methods of the presently disclosed subject matter may be prepared, packaged, or sold in formulations suitable for oral, rectal, vaginal, parenteral, topical, pulmonary, intranasal, buccal, ophthalmic, intrathecal or another route of administration. Other contemplated formulations include projected nanoparticles, liposomal preparations, resealed erythrocytes containing the active ingredient, and immunologically based formulations.

A pharmaceutical composition of the presently disclosed subject matter may be prepared, packaged, or sold in bulk, as a single unit dose, or as a plurality of single unit doses. As used herein, a “unit dose” is discrete amount of the pharmaceutical composition comprising a predetermined amount of the active ingredient. The amount of the active ingredient is generally equal to the dosage of the active ingredient which would be administered to a subject or a convenient fraction of such a dosage such as, for example, one-half or one-third of such a dosage.

The relative amounts of the active ingredient, the pharmaceutically acceptable carrier, and any additional ingredients in a pharmaceutical composition of the presently disclosed subject matter will vary, depending upon the identity, size, and condition of the subject treated and further depending upon the route by which the composition is to be administered. By way of example, the composition may comprise between 0.1% and 100% (w/w) active ingredient.

In addition to the active ingredient, a pharmaceutical composition of the presently disclosed subject matter may further comprise one or more additional pharmaceutically active agents. Particularly contemplated additional agents include anti-emetics and scavengers such as cyanide and cyanate scavengers.

Controlled- or sustained-release formulations of a pharmaceutical composition of the presently disclosed subject matter may be made using conventional technology. A formulation of a pharmaceutical composition of the presently disclosed subject matter suitable for oral administration may be prepared, packaged, or sold in the form of a discrete solid dose unit including, but not limited to, a tablet, a hard or soft capsule, a cachet, a troche, or a lozenge, each containing a predetermined amount of the active ingredient. Other formulations suitable for oral administration include, but are not limited to, a powdered or granular formulation, an aqueous or oily suspension, an aqueous or oily solution, or an emulsion.

As used herein, an “oily” liquid is one which comprises a carbon-containing liquid molecule and which exhibits a less polar character than water.

Liquid formulations of a pharmaceutical composition of the presently disclosed subject matter which are suitable for oral administration may be prepared, packaged, and sold either in liquid form or in the form of a dry product intended for reconstitution with water or another suitable vehicle prior to use.

Liquid suspensions may be prepared using conventional methods to achieve suspension of the active ingredient in an aqueous or oily vehicle. Aqueous vehicles include, for example, water, and isotonic saline. Oily vehicles include, for example, almond oil, oily esters, ethyl alcohol, vegetable oils such as arachis, olive, sesame, or coconut oil, fractionated vegetable oils, and mineral oils such as liquid paraffin. Liquid suspensions may further comprise one or more additional ingredients including, but not limited to, suspending agents, dispersing or wetting agents, emulsifying agents, demulcents, preservatives, buffers, salts, flavorings, coloring agents, and sweetening agents. Oily suspensions may further comprise a thickening agent. Known suspending agents include, but are not limited to, sorbitol syrup, hydrogenated edible fats, sodium alginate, polyvinylpyrrolidone, gum tragacanth, gum acacia, and cellulose derivatives such as sodium carboxymethylcellulose, methylcellulose, hydroxypropylmethylcellulose.

Known dispersing or wetting agents include, but are not limited to, naturally occurring phosphatides such as lecithin, condensation products of an alkylene oxide with a fatty acid, with a long chain aliphatic alcohol, with a partial ester derived from a fatty acid and a hexitol, or with a partial ester derived from a fatty acid and a hexitol anhydride (e.g. polyoxyethylene stearate, heptadecaethyleneoxycetanol, polyoxyethylene sorbitol monooleate, and polyoxyethylene sorbitan monooleate, respectively).

Known emulsifying agents include, but are not limited to, lecithin and acacia. Known preservatives include, but are not limited to, methyl, ethyl, or n-propyl para hydroxy benzoates, ascorbic acid, and sorbic acid. Known sweetening agents include, for example, glycerol, propylene glycol, sorbitol, sucrose, and saccharin. Known thickening agents for oily suspensions include, for example, beeswax, hard paraffin, and cetyl alcohol.

Liquid solutions of the active ingredient in aqueous or oily solvents may be prepared in substantially the same manner as liquid suspensions, the primary difference being that the active ingredient is dissolved, rather than suspended in the solvent. Liquid solutions of the pharmaceutical composition of the presently disclosed subject matter may comprise each of the components described with regard to liquid suspensions, it being understood that suspending agents will not necessarily aid dissolution of the active ingredient in the solvent. Aqueous solvents include, for example, water and isotonic saline. Oily solvents include, for example, almond oil, oily esters, ethyl alcohol, vegetable oils such as arachis, olive, sesame, or coconut oil, fractionated vegetable oils, and mineral oils such as liquid paraffin.

Powdered and granular formulations of a pharmaceutical preparation of the presently disclosed subject matter may be prepared using known methods. Such formulations may be administered directly to a subject, used, for example, to form tablets, to fill capsules, or to prepare an aqueous or oily suspension or solution by addition of an aqueous or oily vehicle thereto. Each of these formulations may further comprise one or more of dispersing or wetting agent, a suspending agent, and a preservative. Additional excipients, such as fillers and sweetening, flavoring, or coloring agents, may also be included in these formulations.

A pharmaceutical composition of the presently disclosed subject matter may also be prepared, packaged, or sold in the form of oil in water emulsion or a water-in-oil emulsion. The oily phase may be a vegetable oil such as olive or arachis oil, a mineral oil such as liquid paraffin, or a combination of these. Such compositions may further comprise one or more emulsifying agents such as naturally occurring gums such as gum acacia or gum tragacanth, naturally occurring phosphatides such as soy bean or lecithin phosphatide, esters or partial esters derived from combinations of fatty acids and hexitol anhydrides such as sorbitan monooleate, and condensation products of such partial esters with ethylene oxide such as polyoxyethylene sorbitan monooleate. These emulsions may also contain additional ingredients including, for example, sweetening or flavoring agents.

A pharmaceutical composition of the presently disclosed subject matter may also be prepared, packaged, or sold in a formulation suitable for rectal administration, vaginal administration, parenteral administration

The pharmaceutical compositions may be prepared, packaged, or sold in the form of a sterile injectable aqueous or oily suspension or solution. This suspension or solution may be formulated according to the known art, and may comprise, in addition to the active ingredient, additional ingredients such as the dispersing agents, wetting agents, or suspending agents described herein. Such sterile injectable formulations may be prepared using a non-toxic parenterally acceptable diluent or solvent, such as water or 1,3 butane diol, for example.

Other acceptable diluents and solvents include, but are not limited to. Ringer's solution, isotonic sodium chloride solution, and fixed oils such as synthetic mono or di-glycerides. Other parentally administrable formulations which are useful include those which comprise the active ingredient in microcrystalline form, in a liposomal preparation, or as a component of a biodegradable polymer systems. Compositions for sustained release or implantation may comprise pharmaceutically acceptable polymeric or hydrophobic materials such as an emulsion, an ion exchange resin, a sparingly soluble polymer, or a sparingly soluble salt.

Formulations suitable for topical administration include, but are not limited to, liquid or semi liquid preparations such as liniments, lotions, oil in water or water in oil emulsions such as creams, ointments or pastes, and solutions or suspensions. Topically administrable formulations may, for example, comprise from about 1% to about 10% (w/w) active ingredient, although the concentration of the active ingredient may be as high as the solubility limit of the active ingredient in the solvent. Formulations for topical administration may further comprise one or more of the additional ingredients described herein.

A pharmaceutical composition of the presently disclosed subject matter may be prepared, packaged, or sold in a formulation suitable for pulmonary administration via the buccal cavity. Such a formulation may comprise dry particles which comprise the active ingredient and which have a diameter in the range from about 0.5 to about 7 nanometers, and preferably from about 1 to about 6 nanometers. Such compositions are conveniently in the form of dry powders for administration using a device comprising a dry powder reservoir to which a stream of propellant may be directed to disperse the powder or using a self-propelling solvent/powder dispensing container such as a device comprising the active ingredient dissolved or suspended in a low-boiling propellant in a sealed container.

In some embodiments, such powders comprise particles wherein at least 98% of the particles by weight have a diameter greater than 0.5 nanometers and at least 95% of the particles by number have a diameter less than 7 nanometers. In some embodiments, at least 95% of the particles by weight have a diameter greater than 1 nanometer and at least 90% of the particles by number have a diameter less than 6 nanometers. Dry powder compositions preferably include a solid fine powder diluent such as sugar and are conveniently provided in a unit dose form.

Low boiling propellants generally include liquid propellants having a boiling point of below 65° F., at atmospheric pressure. Generally, the propellant may constitute 50 to 99.9% (w/w) of the composition, and the active ingredient may constitute 0.1 to 20% (w/w) of the composition. The propellant may further comprise additional ingredients such as a liquid non-ionic or solid anionic surfactant or a solid diluent (preferably having a particle size of the same order as particles comprising the active ingredient).

Pharmaceutical compositions of the presently disclosed subject matter formulated for pulmonary delivery may also provide the active ingredient in the form of droplets of a solution or suspension. Such formulations may be prepared, packaged, or sold as aqueous or dilute alcoholic solutions or suspensions, optionally sterile, comprising the active ingredient, and may conveniently be administered using any nebulization or atomization device. Such formulations may further comprise one or more additional ingredients including, but not limited to, a flavoring agent such as saccharin sodium, a volatile oil, a buffering agent, a surface active agent, or a preservative such as methylhydroxybenzoate. The droplets provided by this route of administration preferably have an average diameter in the range from about 0.1 to about 200 nanometers.

The formulations described herein as being useful for pulmonary delivery are also useful for intranasal delivery of a pharmaceutical composition of the presently disclosed subject matter.

Another formulation suitable for intranasal administration is a coarse powder comprising the active ingredient and having an average particle from about 0.2 to 500 micrometers. Such a formulation is administered in the manner in which snuff is taken i.e., by rapid inhalation through the nasal passage from a container of the powder held close to the nares.

Formulations suitable for nasal administration may, for example, comprise from about as little as 0.1% (w/w) and as much as 100% (w/w) of the active ingredient, and may further comprise one or more of the additional ingredients described herein.

A pharmaceutical composition of the presently disclosed subject matter may be prepared, packaged, or sold in a formulation suitable for buccal administration. Such formulations may, for example, be in the form of tablets or lozenges made using conventional methods, and may, for example 0.1 to 20% (w/w) active ingredient, the balance comprising an orally dissolvable or degradable composition and, optionally, one or more of the additional ingredients described herein. Alternately, formulations suitable for buccal administration may comprise a powder or an aerosolized or atomized solution or suspension comprising the active ingredient. Such powdered, aerosolized, or aerosolized formulations, when dispersed, preferably have an average particle or droplet size in the range from about 0.1 to about 200 nanometers, and may further comprise one or more of the additional ingredients described herein.

As used herein, “additional ingredients” include, but are not limited to, one or more of the following: excipients; surface active agents; dispersing agents; inert diluents; granulating and disintegrating agents; binding agents; lubricating agents; sweetening agents; flavoring agents; coloring agents; preservatives; physiologically degradable compositions such as gelatin; aqueous vehicles and solvents; oily vehicles and solvents; suspending agents; dispersing or wetting agents; emulsifying agents, demulcents; buffers; salts; thickening agents; fillers; emulsifying agents; antioxidants; antibiotics; antifungal agents; stabilizing agents; and pharmaceutically acceptable polymeric or hydrophobic materials. Other “additional ingredients” which may be included in the pharmaceutical compositions of the presently disclosed subject matter are known in the art and described, for example in Remington's Pharmaceutical Sciences, 18th ed . . . 1990, which is incorporated herein by reference.

Typically, dosages of the composition of the presently disclosed subject matter which may be administered to an animal, preferably a human, range in amount from 1 μg to about 100 g per kilogram of body weight of the subject. While the precise dosage administered will vary depending upon any number of factors, including but not limited to, the type of animal and type of disease state being treated, the age of the animal and the route of administration. In one embodiment, the dosage of the compound will vary from about 10 □g to about 10 g per kilogram of body weight of the animal. In another embodiment, the dosage will vary from about 10 mg to about 1 g per kilogram of body weight of the subject.

The composition may be administered to a subject as frequently as several times daily, or it may be administered less frequently, such as once a day, once a week, once every two weeks, once a month, or even less frequently, such as once every several months or even once a year or less. The frequency of the dose will be readily apparent to the skilled artisan and will depend upon any number of factors, such as, but not limited to, the type and severity of the disease being treated, the sex and age of the subject, etc.

The presently disclosed subject matter further provides kits comprising bacteria expressing a peptide or derivative of the bacteria of the presently disclosed subject matter useful for eliciting an immunogenic response, and further includes an applicator and an instructional material for the use thereof.

### Other Embodiments

In some embodiments, the presently disclosed subject matter also provides other systems by which antigens and/or immunogens of interest can be expressed in, on the surface of, or otherwise by bacteria. Thus, it is understood that the autotransporter expression system described herein is not the only way to express antigens. There are many other ways to express antigens and to specifically place them on the surfaces of the bacteria, or even inside the bacteria.

Similarly, the presently disclosed subject matter also provides modified bacteria other than modified E. coli. By way of example and not limitation, other Gram-negative bacteria can also be employed, and other genome reduced strains of other bacteria could also be used. Examples of other bacteria that could be employed include wild type or genome reduced Salmonella or Vibrio. As such, one of ordinary skill in the art could employ the present disclosure as a guide to construct genome reduced versions of other bacterial species for use to express vaccine antigens.

In some embodiments of the presently disclosed subject matter, the modified bacteria can be inactivated. Various methods and approaches for inactivating bacteria for use in immunizations are known to those of skill in the art, and include without limitation use of formalin and/or glutaraldehyde.

Additionally, after review of the instant disclosure one of ordinary skill in the art would also recognize that the purpose of the modified bacterium is primarily to provide a structure in which to provide the immunogen of interest to the immune system of the subject to be immunized. As such, the modified bacterium need not be a fully functional bacterium capable of living, reproducing, etc. As such, in addition to modifications that reduce the genomes of the bacteria, other bacterial derivatives can also be employed. Such derivatives include, but are not limited to minicells, ghost cells, bacterial fragments of cells, including but not limited to isolated outer membrane fragments, blebs, etc.

Furthermore, whereas in some embodiments the presently disclosed subject matter relates to the rapid production of antibodies, the presently disclosed subject matter also relates in some embodiments to the production of prophylactic vaccines for infectious diseases and/or therapeutic vaccines for infectious diseases (such as but not limited to chronic infectious diseases like HIV, other chronic viral diseases. TB, and/or parasitic diseases), therapeutic vaccines for cancer (e.g., off the shelf vaccines directed at know cancer antigens) and custom vaccines designed based on the analysis of the cancer neoantigens for a given patient's cancer (i.e., a personalized anti-cancer vaccine), and therapeutic vaccines for other diseases, particularly diseases involving inflammatory processes, like autoimmune diseases, fibrosis, atherosclerosis, etc.

The antigen can be any desired antigen as would be apparent to one of ordinary skill in the art upon a review of the instant disclosure. In some embodiments, the antigen is derived from a microbe. In some embodiments, the antigen is derived from a cancer. In some embodiments the antigen is derived from a host protein that mediates other diseases or undesirable phenotypes, including in some embodiments autoimmune or inflammatory diseases, or diseases in which the expression of a particular host protein mediates a disease process. In some embodiments, the antigen is derived from a cancer, or a target of an inappropriate or undesirable immune response, or a component of the host immune system, such as (but not exclusively), a host immune system component that, when targeted for destruction or inactivation or activation, alter an undesirable immune response.

In some embodiments, a method for producing an antibody or a desired cell-mediated immune response in a subject is disclosed. In some embodiments, the method comprises providing a modified bacterium in accordance with the presently disclosed subject matter and administering the modified bacterium to a subject in an amount and via a route sufficient to produce an antibody or a desired cell-mediated immune response in the subject against the antigen expressed by the modified bacterium or against cells expressing the antigen. Optionally, the production of the antibody or cell mediated immune response is enhanced in the subject as compared to an immune response produced in a subject by a bacterium of the same strain that has a full complement of expressed genes and that expresses the antigen on its surface. In some embodiments, the administering of the modified bacterium to the subject is intranasally, transmucosally, including but not limited to orally, rectally, and vaginally; subcutaneously, intradermally, intramuscularly, other parenteral routes, or any combination thereof.

In some embodiments, a method for vaccinating a subject in need thereof is provided. In some embodiments, the method comprises providing a vaccine composition of the presently disclosed subject matter and administering the vaccine composition to the subject. In some embodiments, a method for treating a cancer or inappropriate immune responses or expression or production of a deleterious material in a subject in need thereof is provided, the method comprising providing a vaccine composition according to the presently disclosed subject matter and administering the vaccine to the subject. In some embodiments, a method for treating a cancer in a subject in need thereof is provided. In some embodiments, the inappropriate immune response or expression or production of a deleterious material is an autoimmune process, a method for altering the production or expressing of a pathogenic protein, and/or modifying or attacking or killing cells mediating disease. In some embodiments, the vaccine composition is administered orally, rectally, vaginally, intra-nasally, parenterally, intradermally, subcutaneously, or intramuscularly.

In some embodiments, the presently disclosed subject matter provides cancer antigens to immunize the endogenous immune system, i.e., “vaccinating” the subject against their own cancer. In some embodiments, the cancer is a drug resistant cancer or drug sensitive cancer. In some embodiments, the cancer is a cancer characterized by the presence of or as a solid tumor or liquid tumor, or is a cancer of hematologic origin. Thus, in some embodiments, the cancer is selected from the group comprising, but not limited to, pancreatic cancer, breast cancer, prostate cancer, lung cancer, head and neck cancer, non-Hodgkin's lymphoma, acute myelogenous leukemia, acute lymphoblastic leukemia, neuroblastoma, and glioblastoma.

In some embodiments of the presently disclosed subject matter, the modified bacteria can be inactivated. Various methods and approaches for inactivating bacteria for use in immunizations are known to those of skill in the art, and include without limitation use of formalin and/or glutaraldehyde.

Additionally, after review of the instant disclosure one of ordinary skill in the art would also recognize that the purpose of the modified bacterium is primarily to provide a structure in which to provide the immunogen of interest to the immune system of the subject to be immunized. As such, the modified bacterium need not be a fully functional bacterium capable of living, reproducing, etc. As such, alternatively or in addition to modifications that reduce the genomes of the bacteria, other bacterial derivatives can also be employed. Such derivatives include, but are not limited to minicells, ghost cells, bacterial fragments of cells, including but not limited to isolated outer membrane fragments, blebs, etc.

### EXAMPLES

The following Examples provide illustrative embodiments. In light of the present disclosure and the general level of skill in the art, those of skill will appreciate that the following Examples are intended to be exemplary only and that numerous changes, modifications, and alterations can be employed without departing from the scope of the presently disclosed subject matter.

Without further description, it is believed that one of ordinary skill in the art can, using the preceding description and the following illustrative EXAMPLES, make and utilize the compounds of the presently disclosed subject matter and practice the methods of the presently disclosed subject matter. The following EXAMPLES therefore particularly point out embodiments of the presently disclosed subject matter and are not to be construed as limiting in any way the remainder of the disclosure.

### I. Construction of Genome Reduced Bacteria

Representative Approach: Immunization with killed whole cell (KWC) GR E. coli presented intranasally via exp-inc immunization schedules will dramatically decrease the time needed to produced effective custom antibodies. This EXAMPLE relates at least in part preparing genome reduced bacteria.

Gram-negative autotransporters. Gram-Autotransporter (AT) (also termed Autodisplay or Type 5 Secretion System) proteins are a protein family that mediates protein placement into Gram-bacterial outer membranes, with one region anchored in the membrane lipid bilayer and another exposed to the extracellular environment (Henderson et al., 2000; Jose & Meyer, 2007; van Bloois et al., 2011; Nicolay et al., 2015). AT proteins have 3 key domains: An N-terminal signal sequence that directs protein across the inner membrane via a secA mechanism, a C-terminal β-barrel that inserts into the Gram-OM, yielding a pore-like structure, and a central passenger protein domain that transits through the β-barrel pore to be exposed extracellularly, attached to the β-barrel, which remains anchored in the OM. Native passenger protein coding sequence can be replaced with sequence encoding another protein, yielding a recombinant AT protein. The AT thus ‘displays’ recombinant passenger protein to the extracellular environment, anchored in and closely adjacent to the OM lipid bilayer. About 2×10⁵ recombinant proteins can be placed on each cell's surface (Jose & Meyer. 2007). This non-limiting representative approach can be employed in some embodiments of the presently disclosed subject matter.

Genome reduced E. coli. To better understand how the number of gene products on the surface of the E. coli strains changed in the TMUG GR strains, the names of the genes in each deletion (LD5510. LD5119, and LD5125) from the National BioResource Project E. coli Strain website created by the National Institute of Genetics. Japan (https://shigen.nig.ac.jp/ecoli/strain/resource/longDeletion/lddTableInfo) were compiled, with additional gene information from EcoCyc (https://ecocyc.org/), and UniProt (https://www.uniprot.org/), including gene name, protein name, location, function, gene ontology, and other notes about the gene. The information was organized into tables and sorted by deletion. A number of bacterial gene products with an imputed location on the exterior of the cell were eliminated in the GR E. coli. A large number of surface gene products are eliminated in the GR E. coli strains.

An exemplary list of genes that can be deleted from E. coli is presented in Table 2.

TABLE 2
Genes That Were Deleted In The Three Genome Reduced E. Coli Strains*

| | Deleted | Deleted | Deleted | | | | | |
|---|---|---|---|---|---|---|---|---|
| | in | in | in | | | | | |
| Gene | LD5510 | LD5119 | LD5125 | Outer | Integral to | | | Cell |
| Name | (2.4%) | (15.9%) | (29.7%) | Memb¹ | Memb¹ | Memb¹ | Pil² | Surf³ |
| acrZ | 0 | 0 | 1 | 1 | 0 | 0 | 0 | 0 |
| ActP | 0 | 1 | 1 | 0 | 1 | 0 | 0 | 0 |
| AdiC | 0 | 1 | 1 | 0 | 1 | 0 | 0 | 0 |
| aer | 0 | 1 | 1 | 0 | 1 | 0 | 0 | 0 |
| AlsC | 0 | 1 | 1 | 0 | 1 | 0 | 0 | 0 |
| alx | 0 | 1 | 1 | 0 | 1 | 1 | 0 | 0 |
| amiD | 0 | 0 | 1 | 1 | 0 | 0 | 0 | 0 |
| arsB | 0 | 1 | 1 | 0 | 1 | 0 | 0 | 0 |
| artI | 0 | 0 | 1 | 0 | 0 | 1 | 0 | 0 |
| artJ | 0 | 0 | 1 | 0 | 0 | 1 | 0 | 0 |
| basS | 0 | 1 | 1 | 0 | 1 | 0 | 0 | 0 |
| bcsB | 0 | 1 | 1 | 0 | 1 | 1 | 0 | 0 |
| bcsC | 0 | 1 | 1 | 1 | 0 | 0 | 0 | 0 |
| cadB | 0 | 1 | 1 | 0 | 1 | 0 | 0 | 0 |
| clsC | 0 | 1 | 1 | 0 | 0 | 1 | 0 | 0 |
| copA | 0 | 1 | 1 | 0 | 1 | 0 | 0 | 0 |
| csgA | 0 | 1 | 1 | 0 | 0 | 0 | 1 | 0 |
| csgB | 0 | 1 | 1 | 1 | 0 | 0 | 1 | 0 |
| csgE | 0 | 1 | 1 | 1 | 0 | 0 | 0 | 0 |
| csgF | 0 | 1 | 1 | 1 | 0 | 0 | 0 | 0 |
| csgG | 0 | 1 | 1 | 1 | 0 | 0 | 0 | 0 |
| dctA | 0 | 1 | 1 | 0 | 1 | 0 | 0 | 0 |
| dcuB | 0 | 1 | 1 | 0 | 1 | 0 | 0 | 0 |
| dcuS | 0 | 1 | 1 | 0 | 1 | 1 | 0 | 0 |
| dinQ | 0 | 1 | 1 | 0 | 1 | 1 | 0 | 0 |
| dppB | 0 | 1 | 1 | 0 | 1 | 0 | 0 | 0 |
| dppC | 0 | 1 | 1 | 0 | 1 | 0 | 0 | 0 |
| dtpB | 0 | 1 | 1 | 0 | 1 | 0 | 0 | 0 |
| ecpA | 0 | 0 | 1 | 0 | 0 | 0 | 1 | 0 |
| ecpC | 0 | 0 | 1 | 1 | 0 | 0 | 0 | 0 |
| ecpD | 0 | 0 | 1 | 0 | 0 | 0 | 1 | 0 |
| eptA | 0 | 1 | 1 | 0 | 1 | 1 | 0 | 0 |
| eptB | 0 | 1 | 1 | 0 | 1 | 1 | 0 | 0 |
| exuT | 0 | 1 | 1 | 0 | 1 | 1 | 0 | 0 |
| fdnG | 0 | 0 | 1 | 0 | 0 | 1 | 0 | 0 |
| fdrA | 0 | 1 | 1 | 0 | 1 | 0 | 0 | 0 |
| fecA | 1 | 1 | 1 | 0 | 0 | 1 | 0 | 0 |
| fecE | 1 | 1 | 1 | 0 | 0 | 1 | 0 | 0 |
| fimA | 1 | 1 | 1 | 1 | 0 | 0 | 0 | 0 |
| fimD | 1 | 1 | 1 | 1 | 0 | 0 | 0 | 0 |
| fimF | 1 | 1 | 1 | 1 | 0 | 0 | 0 | 0 |
| fimG | 1 | 1 | 1 | 1 | 0 | 0 | 0 | 0 |
| fimH | 1 | 1 | 1 | 1 | 0 | 0 | 0 | 0 |
| fimI | 1 | 1 | 1 | 0 | 0 | 0 | 1 | 0 |
| fiu | 0 | 0 | 1 | 1 | 0 | 0 | 0 | 0 |
| Fsr | 0 | 1 | 1 | 0 | 1 | 0 | 0 | 0 |
| gadB | 0 | 0 | 1 | 0 | 0 | 1 | 0 | 0 |
| gfcD | 0 | 1 | 1 | 1 | 0 | 0 | 0 | 0 |
| gfcE | 0 | 1 | 1 | 1 | 0 | 0 | 0 | 0 |
| glnH | 0 | 0 | 1 | 0 | 0 | 1 | 0 | 0 |
| gltP | 0 | 1 | 1 | 0 | 1 | 0 | 0 | 0 |
| gltP | 0 | 1 | 1 | 0 | 1 | 0 | 0 | 0 |
| gntP | 1 | 1 | 1 | 0 | 1 | 0 | 0 | 0 |
| gutQ | 0 | 0 | 1 | 0 | 0 | 1 | 0 | 0 |
| hdeD | 0 | 1 | 1 | 0 | 1 | 1 | 0 | 0 |
| hslJ | 0 | 0 | 1 | 0 | 0 | 1 | 0 | 0 |
| insH1 | 0 | 1 | 1 | 1 | 0 | 0 | 0 | 0 |
| mdtF | 0 | 1 | 1 | 1 | 0 | 0 | 0 | 0 |
| mdtM | 1 | 1 | 1 | 0 | 1 | 0 | 0 | 0 |
| mdtN | 0 | 1 | 1 | 0 | 1 | 0 | 0 | 0 |
| mdtN | 0 | 1 | 1 | 0 | 1 | 0 | 0 | 0 |
| mdtO | 0 | 1 | 1 | 0 | 1 | 1 | 0 | 0 |
| mdtO | 0 | 1 | 1 | 0 | 1 | 1 | 0 | 0 |
| mdtP | 0 | 1 | 1 | 1 | 0 | 0 | 0 | 0 |
| mdtQ | 0 | 1 | 1 | 1 | 0 | 0 | 0 | 0 |
| melB | 0 | 1 | 1 | 0 | 1 | 0 | 0 | 0 |
| mltB | 0 | 0 | 1 | 1 | 0 | 0 | 0 | 0 |
| nanC | 1 | 1 | 1 | 1 | 1 | 0 | 0 | 0 |
| nrfD | 0 | 1 | 1 | 0 | 1 | 0 | 0 | 0 |
| nrfE | 0 | 1 | 1 | 0 | 1 | 0 | 0 | 0 |
| nrfE | 0 | 1 | 1 | 0 | 1 | 0 | 0 | 0 |
| ompC | 0 | 0 | 1 | 1 | 0 | 0 | 0 | 0 |
| ompG | 0 | 0 | 1 | 1 | 0 | 0 | 0 | 0 |
| ompN | 0 | 0 | 1 | 1 | 0 | 0 | 0 | 0 |
| ompX | 0 | 0 | 1 | 1 | 0 | 0 | 0 | 0 |
| opgB | 1 | 1 | 1 | 0 | 0 | 1 | 0 | 0 |
| pgaA | 0 | 1 | 1 | 1 | 0 | 0 | 0 | 0 |
| pgaB | 0 | 1 | 1 | 1 | 0 | 0 | 0 | 0 |
| phnC | 0 | 1 | 1 | 0 | 0 | 1 | 0 | 0 |
| phnE | 0 | 1 | 1 | 0 | 1 | 1 | 0 | 0 |
| pitA | 0 | 1 | 1 | 0 | 1 | 0 | 0 | 0 |
| prop | 0 | 1 | 1 | 0 | 1 | 0 | 0 | 0 |
| qmcA | 0 | 1 | 1 | 0 | 1 | 1 | 0 | 0 |
| rhsA | 0 | 1 | 1 | 0 | 1 | 1 | 0 | 0 |
| rhsB | 0 | 1 | 1 | 0 | 1 | 1 | 0 | 0 |
| rhsD | 0 | 1 | 1 | 0 | 1 | 1 | 0 | 0 |
| rzoR | 0 | 0 | 1 | 1 | 0 | 0 | 0 | 0 |
| slp | 0 | 1 | 1 | 1 | 0 | 0 | 0 | 0 |
| sstT | 0 | 1 | 1 | 0 | 1 | 1 | 0 | 0 |
| tdcC | 0 | 1 | 1 | 0 | 1 | 0 | 0 | 0 |
| Tsr | 1 | 1 | 1 | 0 | 0 | 1 | 0 | 0 |
| uspB | 0 | 1 | 1 | 0 | 1 | 0 | 0 | 0 |
| wecH | 0 | 1 | 1 | 0 | 1 | 1 | 0 | 0 |
| wza | 0 | 1 | 1 | 1 | 0 | 0 | 0 | 0 |
| xylH | 0 | 1 | 1 | 0 | 1 | 0 | 0 | 0 |
| yaiO | 0 | 0 | 1 | 1 | 0 | 0 | 0 | 0 |
| ybaL | 0 | 1 | 1 | 0 | 1 | 0 | 0 | 0 |
| ybaT | 0 | 1 | 1 | 0 | 1 | 0 | 0 | 0 |
| ybbD | 0 | 1 | 1 | 0 | 1 | 1 | 0 | 0 |
| ybbJ | 0 | 1 | 1 | 0 | 1 | 0 | 0 | 0 |
| ybbM | 0 | 1 | 1 | 0 | 1 | 0 | 0 | 0 |
| ybbP | 0 | 1 | 1 | 0 | 1 | 1 | 0 | 0 |
| ybbW | 0 | 1 | 1 | 0 | 1 | 0 | 0 | 0 |
| ybbY | 0 | 1 | 1 | 0 | 1 | 1 | 0 | 0 |
| ybhC | 0 | 0 | 1 | 1 | 0 | 0 | 0 | 0 |
| ybjC | 0 | 0 | 1 | 0 | 0 | 1 | 0 | 0 |
| yceK | 0 | 1 | 1 | 1 | 0 | 0 | 0 | 0 |
| yddB | 0 | 0 | 1 | 1 | 0 | 0 | 0 | 0 |
| yddL | 0 | 0 | 1 | 1 | 0 | 0 | 0 | 0 |
| ydeQ | 0 | 0 | 1 | 0 | 0 | 0 | 1 | 0 |
| ydeR | 0 | 0 | 1 | 0 | 0 | 0 | 1 | 0 |
| ydeS | 0 | 0 | 1 | 0 | 0 | 0 | 1 | 0 |
| ydeT | 0 | 0 | 1 | 1 | 0 | 0 | 0 | 0 |
| yehA | 0 | 1 | 1 | 0 | 0 | 0 | 1 | 0 |
| yehB | 0 | 1 | 1 | 1 | 0 | 0 | 0 | 0 |
| yehD | 0 | 1 | 1 | 0 | 0 | 0 | 1 | 0 |
| yehR | 0 | 1 | 1 | 0 | 0 | 1 | 0 | 0 |
| yehU | 0 | 1 | 1 | 0 | 1 | 1 | 0 | 0 |
| yehW | 0 | 1 | 1 | 0 | 1 | 0 | 0 | 0 |
| yehY | 0 | 1 | 1 | 0 | 1 | 0 | 0 | 0 |
| yejO | 0 | 0 | 1 | 1 | 0 | 0 | 0 | 0 |
| yfaZ | 0 | 0 | 1 | 1 | 0 | 0 | 0 | 0 |
| yfjV | 0 | 1 | 1 | 0 | 1 | 0 | 0 | 0 |
| ygjI | 0 | 1 | 1 | 0 | 1 | 1 | 0 | 0 |
| ygjQ | 0 | 1 | 1 | 0 | 1 | 1 | 0 | 0 |
| ygjR | 1 | 1 | 1 | 0 | 0 | 0 | 0 | 1 |
| ygjV | 0 | 1 | 1 | 0 | 1 | 1 | 0 | 0 |
| yhaH | 0 | 1 | 1 | 0 | 1 | 1 | 0 | 0 |
| yhaI | 0 | 1 | 1 | 0 | 1 | 1 | 0 | 0 |
| yhaO | 0 | 1 | 1 | 0 | 1 | 0 | 0 | 0 |
| yhhJ | 0 | 1 | 1 | 0 | 1 | 0 | 0 | 0 |
| yhiD | 0 | 1 | 1 | 0 | 1 | 1 | 0 | 0 |
| yhiM | 0 | 1 | 1 | 0 | 1 | 1 | 0 | 0 |
| yhjD | 0 | 1 | 1 | 0 | 1 | 1 | 0 | 0 |
| yhjE | 0 | 1 | 1 | 0 | 1 | 0 | 0 | 0 |
| yhjG | 0 | 1 | 1 | 0 | 1 | 1 | 0 | 0 |
| yhjK | 0 | 1 | 1 | 0 | 0 | 1 | 0 | 0 |
| yhjV | 0 | 1 | 1 | 0 | 1 | 0 | 0 | 0 |
| yhjX | 0 | 1 | 1 | 0 | 1 | 0 | 0 | 0 |
| yhjY | 0 | 1 | 1 | 0 | 0 | 1 | 0 | 0 |
| yiaA | 0 | 1 | 1 | 0 | 1 | 1 | 0 | 0 |
| yiaB | 0 | 1 | 1 | 0 | 1 | 1 | 0 | 0 |
| yiaD | 0 | 1 | 1 | 0 | 1 | 0 | 0 | 0 |
| yiaM | 0 | 1 | 1 | 0 | 1 | 1 | 0 | 0 |
| yiaN | 0 | 1 | 1 | 0 | 1 | 0 | 0 | 0 |
| yiaT | 0 | 1 | 1 | 1 | 0 | 0 | 0 | 0 |
| yiaV | 0 | 1 | 1 | 0 | 1 | 0 | 0 | 0 |
| yiaW | 0 | 1 | 1 | 0 | 1 | 0 | 0 | 0 |
| yibH | 0 | 1 | 1 | 0 | 1 | 0 | 0 | 0 |
| yibI | 0 | 1 | 1 | 0 | 0 | 1 | 0 | 0 |
| yjcE | 0 | 1 | 1 | 0 | 1 | 0 | 0 | 0 |
| yjcH | 0 | 1 | 1 | 0 | 1 | 1 | 0 | 0 |
| yjdF | 0 | 1 | 1 | 0 | 1 | 1 | 0 | 0 |
| yjdL | 0 | 1 | 1 | 0 | 1 | 0 | 0 | 0 |
| yjdO | 0 | 1 | 1 | 0 | 1 | 1 | 0 | 0 |
| yjhE | 1 | 1 | 1 | 0 | 0 | 1 | 0 | 0 |
| yjhP | 1 | 1 | 1 | 0 | 1 | 0 | 0 | 0 |
| yjiG | 1 | 1 | 1 | 0 | 1 | 0 | 0 | 0 |
| yjiH | 1 | 1 | 1 | 0 | 1 | 0 | 0 | 0 |
| yjiJ | 1 | 1 | 1 | 0 | 1 | 0 | 0 | 0 |
| yjiK | 1 | 1 | 1 | 1 | 1 | 0 | 0 | 0 |
| yjiN | 1 | 1 | 1 | 0 | 1 | 0 | 0 | 0 |
| yjiN | 1 | 1 | 1 | 0 | 0 | 1 | 0 | 0 |
| yjiY | 1 | 1 | 1 | 0 | 0 | 1 | 0 | 0 |
| yjjL | 1 | 1 | 1 | 0 | 0 | 1 | 0 | 0 |
| yliI | 0 | 0 | 1 | 1 | 0 | 0 | 0 | 0 |
| ynbC | 0 | 0 | 1 | 0 | 0 | 1 | 0 | 0 |
| ynbC | 0 | 0 | 1 | 0 | 0 | 1 | 0 | 0 |
| ynbC | 0 | 0 | 1 | 0 | 0 | 1 | 0 | 0 |
| yncD | 0 | 0 | 1 | 1 | 0 | 0 | 0 | 0 |
| yohC | 0 | 1 | 1 | 0 | 1 | 0 | 0 | 0 |
| yohO | 0 | 1 | 1 | 0 | 1 | 1 | 0 | 0 |
| ypjA | 0 | 1 | 1 | 1 | 0 | 0 | 0 | 0 |
| yqjA | 0 | 1 | 1 | 0 | 1 | 0 | 0 | 0 |
| yqjE | 0 | 1 | 1 | 0 | 1 | 1 | 0 | 0 |
| yqjF | 0 | 1 | 1 | 0 | 1 | 1 | 0 | 0 |
| yqjI | 0 | 1 | 1 | 0 | 0 | 0 | 1 | 0 |
| ytcA | 0 | 1 | 1 | 0 | 1 | 1 | 0 | 0 |
| ytcA | 0 | 1 | 1 | 0 | 1 | 1 | 0 | 0 |
| ythA | 1 | 1 | 1 | 0 | 0 | 1 | 0 | 0 |

*Genes are listed by locus name, with the presence (=1) or absence (=0) for each deleted mutant strain and for each location in the bacteria.
¹membrane;
²pilus;
³cell surface

Production of Gram-AT recombinant expression systems for rapid Ab production immunizations. In preliminary experiments, plasmid pRIAIDA, which has a rhamnose inducible AIDA-I Gram-AT expression cassette for expression optimization, with a cloning site, flanked by a trypsin site to evaluate surface expression was constructed. In initial experiments, a nucleic acid sequence encoding a widely used influenza virus HA tag (YPYDVPDYA; SEQ ID NO: 91) was inserted into the surface expression cassette to make pRIAIDA-HA. A trypsination experiment confirmed that that HA immunotag resided on the exterior of the bacteria when expressed in an AIDA-I expression cassette that includes a trypsin site in the coding sequence.

Intranasal immunization. Intranasal immunization has a number of significant advantages (Davis, 2001; Jabbal-Gill, 2010; Zaman et al., 2013; Riese et al., 2014; Nizard et al., 2017; Yusuf & Kett, 2017). Intranasal immunization leads to the direct exposure in the nasal mucosa of M cells and dendritic cells to the immunogen. In addition, there are abundant nasopharyngeal lymphoid tissues with large numbers of other antigen presenting cells, like macrophages, and many T cells and B cells. Intranasal immunization can induce potent tissue-resident effector and effector memory CD8+ T cell immunity (Morabito et al., 2017). Intranasal immunization may also be particularly effective in producing antibodies in other body compartments if, for example, secretory mucosal antibodies may be useful (Russell. 2002). In addition, since most pathogens enter the host across mucosal surfaces, if the hypothesis that the best way to rapidly elicit potent antibody response is to mimic a significant pathogen threat, mucosal immunization can elicit better immune responses than a more traditional parenteral route.

Evaluation of antibody binding to the GR E. coli expressing a test immunogen on their surfaces and the ability of the GR E. coli expressing a test immunogen on their surfaces to elicit immune responses. As an initial step for a new method employing a combination of strategies to enhance immunogenicity for the production of custom Abs, pRIAIDA-HA was transformed into wt parental E. coli and three GR E. coli from the TMUG collection. Binding of a commercial anti-HA mAb to the bacteria was confirmed via flow cytometry. It was determined that the ability of the bacteria to bind the anti-HA mAb increased significantly as the fraction of the genome deleted increased. The ability of the GR E. coli to elicit an immune response was tested. Mice were immunized intranasally with 10⁸ formalin fixed bacteria. After 2 weeks, blood was collected from the immunized mice and the sera tested using an ELISA, with commercial anti-HA mAb as a standard. It was found that intranasal immunization with GR E. coli expressing the test immunogen on their surfaces could elicit the production of large amounts of Abs in only 2 weeks, and that the ability of the bacteria expressing the test antigen to elicit production of the Ab increased substantially in bacteria with more genes deleted. Preliminary data using immuno-dot blots indicated that the same amount of HA was produced per cell regardless of extent of the genome reduction, which supported the hypothesis that the increased binding and immunogenicity was due to either increased antigen accessibility in the GR strains, or an absence of immunoinhibitory surface structures or both.

Exponentially increasing (exp-inc) immunization schedules. Recent studies comparing alternative immunization schedules have shown that repeated immunizations with exp-inc amounts of immunogens can yield a dramatically improved immune response, with a >10-fold increase in Ab concentrations (Tam et al., 2016). The hypothesized reason for the enhanced response is that the exp-inc antigen dosing schedule creates signals to the host immune system similar to those initiated by serious infections that threaten the host, with the enhanced response promoted by prolonged, greater amounts of antigen present in lymph nodes acting to improve antibody maturation. Exp-inc immunization has also been observed to induce more Tfh cells and germinal center B-cells. This non-limiting representative approach can be employed in some embodiments of the presently disclosed subject matter.

Scientific Premise. The immune system responds to threatening pathogens by rapidly producing a potent humoral immune response. The presently disclosed subject matter provides new methods that enable the rapid production of useful polyclonal Abs that can be employed in wide range of biomedical research projects. It has been demonstrated that a substantial antibody response against a test antigen can be produced in only two weeks. This EXAMPLE is aimed at extending and optimizing the work, and demonstrating that these new procedures can produce a rapid, useful polyclonal Ab response in rabbits, the typical species employed in producing useful amounts of polyclonal Abs for biomedical research. Table 3 summarizes the innovations that are employed in the work, along with the different rationales for their use.

The preliminary data showed that the production of antigen via gene synthesis in a Gram-AT expression vector in GR E. coli with intranasal immunization can elicit a strong Ab response in 2 weeks.

TABLE 3
Summary Of Features Employed And Proposed In The New Rapid Ab Production Method

| Feature | Comments |
|---|---|
| Antigen production using synthetic | Rapid immunogen production; Increased immunogenicity |
| biology | through production of tandem multimeric antigens; |
| | Increased immunogenicity through amino acid |
| | sequence modifications, Inherent fusion to carrier |
| | protein |
| Surface expression via autotransporter | Direct interaction with B-Cell receptors plus |
| (Type 5 secretion system) | processing/presentation by APCs; Auto-adjuvanting |
| expression cassette | through presence of multiple PAMPs on bacteria |
| Genome reduced E. coli | Fewer bacterial proteins interfering with vaccine antigen- |
| | immune system interactions; enhanced exposure of the |
| | immunizing antigen; loss of components masking |
| | bacterial surface antigens from the immune system |
| Intranasal immunization | Direct access to mucosal compartment rich in dendritic |
| | cells and APCs; Needle-free immunization: Induction |
| | of mucosal immunity |
| Exponential increasing immunogen dose | Mimics signals to the immune system that the immunogen |
| | comes from a serious pathogen threat |

### Research Methods

Overview. The presently disclosed procedure rapidly produces custom polyclonal Abs. It also indicates the key areas of additional optimization that are pursued to further enhance the ability of the new procedure to rapidly produce custom polyclonal Abs. Please also note that even though preliminary work strongly supports that intranasal immunization with GR E. coli expresses an antigen-of-interest on its surfaces using a Gram-AT expression cassette, additional research can be performed to enable effective, rapid production of custom Abs. This work includes defining the kinetics of the Ab response beyond 2 weeks, a careful analysis of the Ab subtypes to maximize the utility of the custom Abs produced using the new procedure, and work to determine whether induction of the Ab response could be further enhanced. To make the new procedure broadly useful for biomedical research, that the procedure can elicit production of custom Abs in a species large enough to make useful quantities of custom Abs, such as rabbit, is also demonstrated.

### 1. Optimize Genome Reduced Bacterial Immunization for Rapid Antibody Production in Mice.

A series of methods are tested to further optimize rapid production of Abs in mice, then test the most effective of those methods in rabbits, confirming the utility of the new technology to rapidly produce custom Abs useful for research.

Design of expression cassettes, surface expression and bacterial toxicity testing. An E. coli codon-optimized version of the HA immunotag coding sequence was cloned into pRIAIDA, transforming the derivative, into wt non-deleted and GR (2.4%, 15.9%, 29.7%) E. coli (Hashimoto et al., 2005; Kato & Hashimoto, 2007), verifying expression by flow cytometry and surface expression by immunoblot with and without trypsin treatment. Since HA is short, highly immunogenic immunotag, a second, larger immunotag is also used to verify that the proposed new Ab production procedure is effective. The ability of the procedure to elicit production of Abs against another model immunogen is also tested. For this second immunogen GFP (MW 27 kDa) is chosen, which is useful for confirming surface expression on the bacteria with IF and flow cytometry. GFP has also been used as a test immunogen in many studies, including studies that showed that E. coli-derived outer membrane vesicles engineered to contain GFP elicited better immune responses than GFP alone (Chen et al., 2010). In addition, excellent, well-tested reagents, both recombinant protein and mAbs, are commercially available for GFP (e.g. Abcam, SigmaAldrich, ThermoFisher). The AIDA-I AT has been shown to transport GFP and GFP fusions to bacterial surfaces (Li et al., 2008). For the GFP (and any other alternate immunogen constructs), optimum rhamnose induction of expression is determined, following bacterial growth by OD₆₀₀ to determine the maximum expression possible without compromising bacterial replication. The number of immunogen molecules on the surface of each bacteria is determined using immunoblots on bacterial extracts and immunogen protein standards. A goal is 2×10⁵ molecules/bacteria, which has been achieved with other immunogens, and which has been achieved with other antigens, including HA. GFP was also chosen as a widely used marker in biological research that has no clinical use.

Production of immunogens—whole GR E. coli. The methods will be those that yielded the preliminary data. Bacteria are grown in LB broth with optimized rhamnose induction, monitoring growth by OD₆₀₀. Bacteria are collected by centrifugation, washed in HBSS, without Ca²⁺/Mg²⁺, 10% formalin is added to a final 0.2% concentration, and incubated at 37° C., for 1 hour with shaking. Aliquots are stored at −80° C., in PBS/10% glycerol, confirming immunogen expression by flow cytometry on thawed stocks. Surface expression was confirmed using a trypsinization procedure either with immunoblots, or flow cytometry (e.g., for GFP).

Evaluating immunogenicity—humoral immunity. A sandwich ELISA was constructed by binding commercial anti-HA mAb (Invitrogen) to blocked, streptavidin-coated strips (Pierce), followed by incubation with commercially produced HA peptide, followed by commercial anti-HA mAb (Invitrogen) with HRP-conjugated goat anti-mouse secondary antibody, assayed using the Tropix CSPD luminescence system. In those experiments, it was found that anti-HA Abs could be detected to the 1 ng/ml level, below the physiologic level of antibodies against vaccine antigens following vaccination (Huang et al., 2012).

For the ELISAs on the sera from the immunized animals, a sandwich ELISA is employed. To develop assays, commercially available HA and GFP proteins were employed as standards. IgG subclasses (IgG1. IgG2a. IgG (Total)). IgA, and IgM are quantitated with HRP-conjugated anti-mouse class and subclass mAbs, with the 1-Step slow TMB-ELISA substrate, or as an alternative, the Tropix CSPD luminescence system, although it is not believed that the added sensitivity of the chemiluminescent assay will be required.

Evaluating immunogenicity—cell-mediated immunity. While the deliverable for this project is an improved method to rapidly produce custom Abs, it may be helpful in evaluating, comparing, and optimizing the different procedures to have information on the cell mediated immune responses elicited by the different immunization procedures. To characterize the cell-mediated immune responses. ELISpot assays are performed according to the kit manufacturer's instructions (Mabtech). Spleen cells (5×10⁶ cells/mL) obtained at the conclusion of the experiment will be plated and stimulated in the presence (or not) of HA (or GFP peptide mix) and incubated for 24 hours. Plates are washed and incubated with biotinylated detection antibody, then incubated with streptavidin-ALP, followed by substrate solution (BCIP/NBT-plus).

In addition to ELIspot assays, assessment of antigen specific T-cell frequency, proliferative capacity, cell surface immunophenotyping, and intracellular cytokine production profiles are determined by flow cytometry to characterize T cell responses. Cells (Maecker et al., 2005) challenged with immunogens are evaluated with a polychromatic (12 color) flow cytometric panel to determine frequencies of HA positive cells, and characterize T_{EM} or T_{CM} phenotypes, since T_{CM} cells VS. T_{EM} cells (Seder & Ahmed, 2003; Klebanoff et al., 2005; Klebanoff et al., 2006), which might imply the induction of longer-term immunity, might be helpful for additional boosting and distinguishing between different immunization strategies. For defining T_{EM} and T_{CM} cells, various markers recommended by the Human Immunology Project (Maecker et al., 2012) are employed including CD3, CD4, CD8, CCR7, CD45RA. To assess functionality of the response, intracellular cytokine production and proliferative capacity are measured, and intracellular cytokine staining for IL2. IFN-γ, and TNF-α is performed. Proliferation is evaluated by intracellular staining for Ki-67, which is expressed in cells in S. G₂, and M phases, but not G₀ or G₁ (Gerdes et al., 1984). Fixable amine reactive viability dye is used to eliminate evaluation of dead cells. Non-T cells are identified with a cocktail including anti-CD19, CD14, CD16, CD56, CD11c and CD11b Abs. At least 100,000 events are acquired on a BD 4 laser 17 color FORTESSA™ flow cytometer. Data are analyzed using FlowJo10 (Treestar) software with this gating strategy: 1) Gate for single cells using a FSC area vs. FSC height plot. 2) Gate for live T cells using a CD3 vs. Viability/dump channel (lineage cocktail) plot. 3) Gate for antigen specific CD8+ T cells using CD8 vs. Pentamer plot. 4) Phenotype T_{EM} and T_{CM} using CCR7 vs. CD45RA plot, and 5) Evaluate intracellular cytokine profiles and Ki-67 positivity within the T_{EM} and TOM cell populations.

Immunizations—immunization methods. Immediately before immunization. KWC preparations are thawed on ice and wash in PBS. For the single (non exp-inc) intranasal immunizations. 6 week old mice (CB6F1/J. Jackson) mice are immunized intranasally with 10⁸ cells in 50 ul PBS, boosting at 2 weeks.

Later, immunizations done at a single time with 10⁸ cells are compared with an exponential dose escalation, using doses of 10⁸, 3×10⁸, and 10⁹ cells on alternating days.

For the experiments with the GR E. coli, since the aim of the project is to develop an accelerated immunization schedule, a rapid schedule with an initial prime and a boost after 2 weeks is employed. Sera is collected at baseline, before immunization, 2 weeks after immunization, before the boost, and 2 weeks after the boost, at which time the mice are euthanized and larger blood volumes are collected by terminal bleed, along with spleens for the isolation of spleen mononuclear cells for the assays for cell mediated immunity (see below).

As controls (see below for more details on the head-to-head comparisons), mice are immunized SC with the same doses of bacteria used in the intranasal experiments, and with commercially purchased recombinant immunogen protein (HA. GFP), using an initial immunization with protein immunogen emulsified in CFA, followed by boosts in IFA, a standard immunization schedule, to elicit anti-protein humoral immune responses (Greenfield. 2013). The schedule will be based on a typical schedule for the production of custom polyclonal Abs against a protein immunogen.

Mice are observed daily, recording water and food consumption, abnormal clinical observations, mortality, and weekly weights. Blood is sampled and serum stored at baseline, then before boost and 2 weeks after boost, with terminal bleed via cardiac puncture. Serum is stored and spleen mononuclear cells are harvested and cryopreserved for the ELIspot and intracellular cytokine staining procedures.

Evaluating immunogenicity—analytic considerations and experiment planning. For each immunization strategy, groups of 6 animals are employed. The power analysis, dictating 6 animals/group, is based on a two-sided, two-sample t-test with hypothesized relative effect sizes. |μ1-μ2|/s, yielding at least 90% power to detect a relative effect size. |μ1-μ2|/s, of 2.7 between any two groups with a familywise type I error of 0.05, which accounts for multiple comparisons although, given the preliminary data, the observed effects are expected to be far in excess of this threshold. Using groups of 6 animals will therefore provide better than adequate power to identify the better immunization strategy in each pair.

For each of these comparisons, the primary evaluation to advance a particular immunization procedure to the next stage comparison will be the production of IgG (Total) against the immunogen, but the ability of the new procedures to elicit production of specific IgG subtypes. IgM, and IgA, and CMI is also taken into account.

The preliminary experiments presented above are also repeated in an expanded form, with additional controls. The immune responses of mice immunized with wt E. coli (ME5000, the TMUG parental strain, as well as a commonly used E. coli strain. MG1655; Hashimoto et al., 2005; Kato & Hashimoto, 2007), transformed with the immunogen expressing plasmids and bacteria not transformed with plasmids as negative controls, are compared. GR E. coli TMUG strains, with genome reductions of 2.4%, 15.9%, and 29.7%, are compared employing the more extensive immune assays described above.

As an initial comparator for traditional immunization procedures. SC immunizations with the bacteria expressing the immunogens is employed, and SC immunizations with commercial recombinant HA and GFP. For the protein antigens the CFA prime. IFA for boosts (Greenfield. 2013) are employed. In addition, for these comparison experiments, to maximize the ability of a standard immunization procedure to elicit a good humoral immune response, the HA and GFP test immunogens are conjugated to KLH using a widely available kit (e.g., AAT Bio ReadiLink KLH Conjugation Kit or Novus/Techne Imm-Link for carboxyl conjugation since the HA immunotag does not include S or M).

Evaluating immunogenicity—pairwise comparisons of immunization procedure enhancements. The preliminary data indicated, in a type of “dose-response” study, that immunization with bacteria expressing recombinant immunogen on the surface of the most highly GR E. coli strain (29.7% deleted) elicited much better immune responses than the wt or less deleted bacteria. Assuming the repeat experiments recapitulate the preliminary results, responses elicited by the largest deletion. 29.7%. GR E. coli expressing the immunogens and administered intranasally, pairwise, are compared with: a) SC immunization with the protein immunogen using the CFA prime/IFA boost regimen, b) SC immunization with wt E. coli expressing the immunogen, and c) SC immunization with the 29.7% GR E. coli expressing the immunogens. Given the preliminary results, and the known benefits of intranasal immunization described above, it is believed that the intranasal immunization with the GR E. coli vector will yield the best immune responses.

The product of the experiments above are a vaccination regimen that is used in the experiments below to show that the new procedures can rapidly yield the production of usable quantities of custom Abs.

### 2. Demonstrate Rapid Production of Polyclonal Antibodies in Rabbits Using Optimized Immunization Procedures

Overview. Since an aspect of this EXAMPLE pertains to procedures that enable the rapid production of custom Abs broadly useful for biomedical research, the following experiments are provided to demonstrate the utility of the new procedures by showing that we can rapidly produce custom Abs in the species most commonly used to produce new custom polyclonal Abs, the rabbit. Having optimized and refined the new immunization procedure in the mouse experiments above, it is then shown that the optimized procedure is effective in the rabbit. The two immunization procedures that yielded the highest mean Ab concentration elicited 14 days after the boost immunization are determined and those procedures are used to immunize the rabbits, comparing the two best procedures with each other and with SC immunizations with killed whole bacteria vaccines, and with SC immunization using purified immunogen proteins.

In these experiments, a focus is on the intranasal immunization of rabbits, in comparison with the typical CFA/IFA SC protein prime/boost schedules, given our preliminary results. If immunization via another route with the GR E. coli proves to be more effective, then that route is used. In any case, given the preliminary results, with the evidence for greatly enhanced accessibility of the surface expressed immunogen on the GR E. coli, it is believed that expression of protein immunogens on the surfaces of GR E. coli will almost certainly elicit enhanced immune responses compared to non-GR bacteria or native protein.

New Zealand White rabbits are used, since this breed is the most commonly used breed to produce custom antibodies and is of medium-large size, enabling reasonable blood volume sampling.

Rabbit immunizations. Groups of 6 rabbits are immunized with the statistical considerations described above. For the rabbit studies, due to the larger size of the animals. 10⁹ cells are used for single immunizations. If the exp-inc immunizations in the mice yield significantly better responses, for the rabbits 10⁹, 3×10⁹, and 10¹⁰ cells per dose every other day are used.

If it is determined in the initial mouse studies that intranasal immunization yields the best immune responses, which is expected given the preliminary data, this route is also used for the rabbit confirmatory studies. Intranasal immunization has been commonly used in rabbit studies, typically in a volume of 0.5 ml, administered by dripping by pipette into the nares of the rabbit held in an inverted position (Shoemaker et al., 2005; Oliveira et al., 2007).

For the comparison, rabbit SC immunizations are used, employing CFA/IFA prime-boost methods widely described in detail in standard reference works (Greenfield. 2013), which procedures are used in conducting the SC immunizations as reference for comparing the new GR E. coli methods, using the HA and GFP (conjugated, see above) test immunogens. For the comparison conventional protein immunizations. 200 μg for the prime and 100 μg for each boost are used. Boosts are used every 2 weeks for a total of 5 times, with both the conventional procedure and new GR E. coli-based immunizations.

Rabbit blood sampling. Blood samples are obtained via marginal car vein venipuncture. Assuming a ˜5 Kg size for the typical New Zealand White, with a blood draw volume limit of 1% of body weight every 2 weeks, it is sampled ˜5 ml at baseline, then after 2 weeks, before boosting, and 2 weeks after each boost at 2 week intervals. After the final boost it is continued to sample at 2 wk intervals for a total 6 months, to evaluate any additional maturation in the immune responses and to establish that the blood draws from the rabbits immunized according to the accelerated procedure can produce commercially and biomedically useful quantities of sera over a long time. At the end of the experiment, a terminal bleed is conducted following euthanasia to confirm that a useful maximum amount of sera from the rabbit immunized can be produced using the procedures.

Testing rabbit sera elicited by the new procedures. A comparison between the standard procedure and the new procedure is Ab concentrations elicited against the HA and GFP immunogens, comparing the kinetics of Ab development in the conventional and new GR E. coli procedures. Humoral immunity is evaluated using the sandwich ELISA methods described above, using the appropriate HRP-conjugated anti-rabbit IgG (Total). IgG1. IgG2a, and IgA and IgM antibodies (Abcam. ThermoFisher. Sigma) in place of the anti-mouse used above. A comparison between the new and conventional procedures is made at the 2-week and 4-week times after the first prime immunization, and an evaluation is made of the comparison between the sera elicited from the rabbits using the new procedure at 2 and 4 weeks with the sera elicited from the rabbits using the conventional procedure at end of the experiment. 16 weeks after the prime immunization.

Testing rabbit cellular immune responses elicited by the new procedures. While a goal of this EXAMPLE is the rapid production of biotechnologically effective antisera, the ability of the GR E. coli to stimulate cell-mediated immunity is also compared. The same techniques as described above for the study of mouse cell-mediated immunity are used, modifying the procedures to use the appropriate antibodies and reagents directed against the equivalent rabbit markers. For the rabbit experiments, given that one can obtain much larger blood volumes, peripheral lymphocytes are used, and the ELIspot assays are conducted on blood from each draw, not just the terminally obtained spleen cells.

Functional evaluation of sera elicited in rabbits using the new procedures. Since a goal of this EXAMPLE is to develop a procedure to rapidly elicit highly effective custom polyclonal antibodies for use in a wide variety of biological research projects, it is confirmed that the sera elicited against the test immunogens in the rabbit are functional for key techniques in which custom antisera are used. These include immunoblotting, flow cytometry, and immunofluorescence microscopy. For each of these applications the polyclonal Abs produced in the new procedure are compared with commercially available rabbit polyclonal Abs (e.g. Abcam). For the immunoblotting evaluation, it is confirmed that the elicited rabbit sera can detect the HA and GFP antigens produced in the bacteria, run in parallel with commercially produced GFP and HA-immunotagged protein standards (e.g., Abcam. ThermoFisher), using commercial HRP-conjugated anti-rabbit secondary Ab. For the flow cytometry experiments the ability of the elicited rabbit antisera to bind the bacteria is compared, but since many Abs in the polyclonal sera will likely be directed against bacterial antigens, an important test will be for the ability of the sera to stain mammalian cells expressing the test antigens. For the flow cytometry and IF experiments commercially available stable cell lines expressing GFP and HA-fusion proteins (e.g. GenTarget, R & D/Biotechne. ATCC) are employed, using standard protocols recommended the by vendor of the commercial polyclonal rabbit Abs (e.g. https://www.abcam.com/protocols/indirect-flow-cytometry-protocol: https://www.abcam.com/protocols/immunocytochemistry-immunofluorescence-protocol) with commercially available anti-rabbit (e.g. ThermoFisher Invitrogen Goat anti-rabbit-Alexa 594). In the case of the flow cytometry studies using the GFP cell lines, it can be compared directly signal from GFP gating with signal gating on the fluor labeling the anti-rabbit secondary Ab.

In summary, an aspect of this project is that surface expression of immunogen on the GR E. coli are particularly immunogenic. It is believed that the preliminary data indicate it is possible to rapidly elicit effective polyclonal Abs. An additional approach is taken to enhancing immune response: exp-inc immunization schedule. There are additional methods to increase immunogenicity, which can be tested, if needed. Additionally, the bacteria can be administered together with additional adjuvant, such as cholera toxin B subunit, AS03, AS04, and/or MF59, although care should be taken in using lipophilic adjuvants that they do not excessively damage the bacterial outer membrane that holds the immunogen.

In summary, the first experiments are devoted to optimizing the immunization protocols in mice. The immunogen-expressing plasmids are constructed and tested, and a series of head-to-head comparisons of routes, immunogens, and schedules (single dose vs, exp-inc), comparing the induction primarily of humoral immune responses, are performed. Thereafter, how the best, optimized immunization methods developed in the mouse models work to rapidly produce custom polyclonal Abs in the rabbit, the principle source for custom polyclonal Abs for research purposes, are determined.

An immediate future downstream biotechnological application is the accelerated production of mouse mAbs. Antigen production, purification, conjugation, immunization, and boosting for the production of mouse mAbs also occupy considerable time prior to fusion and hybridoma production. Shortening this time from months to weeks would significantly accelerate production of mouse mAbs to a similar extent that will occur with the production of custom polyclonal Abs.

A very rapid synthetic biology recombinant bacterial vaccine platform provides utility for many clinical purposes, from the rapid development of prophylactic vaccines for infectious diseases to custom tumor antigen-directed cancer immunotherapy. From a basic biological research perspective, understanding why the GR E. coli are such highly effective immunogens could yield insights helpful in many areas, including whether the enhanced immunogenicity is the result of increased immunogen accessibility, or whether some of the gene products removed from the surfaces of the GR E. coli blunt the host response against the immunogens expressed on the bacterial surface, and/or whether if some of the gene products do blunt the host immune response, what are the responsible mechanisms. While it is not desired to be bound by any particular theory of operation, understanding such mechanisms offers insights into the processes governing the assembly and maintenance of a wide range of host microbial communities.

### II. Maximal Expression and Surface Exposure of a Test Ag, the SARS-CoV-2 FP, in a KWC grEc Vaccine Platform

Modifying the surface-expressed test Ag adding hydrophilic amino acids to the N-terminal, by making a multimeric version, and by adding hydrophilic amino acids in between multimers of the Ag will enhances Ag exposure and immunogenicity.

This is done by employing a canonical bioengineering DBTL approach to iteratively test, via gene synthesis, with cloning into the expression vector, the modifications to enhance the production and exposure of a test antigen, the SARS-CoV-2 FP, on the surfaces of Genome-reduced E. coli (grEc).

### III. Compare the Immunogenicity and Protection Against Experimental Infection of an Optimally Expressed and Exposed SARS-CoV-2 FP Test Ag with the Prototypical 1-Mer FP Test Ag

Vaccines made from grEc expressing the SARS-CoV-2 FP optimized for maximal expression and exposure are more immunogenic than prototypical FP monomer KWC grEc vaccine. Vaccination with the improved Ag elicits a more potent and protective immune response.

This is achieved by vaccinating mice with the grEc KWC original 1-mer FP (Maeda et al. 2021) and 3 synthetic biologically enhanced vaccines (mod 1, mod 2, mod 3) with the best Ab binding, via intramuscular (IM). Oral (PO), and Intranasal (IN) routes, and evaluate immune responses by anti-FP antibody ELISA, and cellular immune responses by ELIspot and CD4 and CD8 T cell responses by intracellular cytokine staining. Testing of SARS-CoV-2 neutralization titers in a Vero cell plaque reduction assay and comparing the ability of the KWC and minicell grEc vaccines to protect against challenge infection is assessed in the K18 ACE2 mouse model challenge experiment.

FP vaccines are made using modifications that enhance Ag exposure. Modified 5-mer versions of the vaccine are more immunogenic than the original monomeric version, eliciting higher antibody levels per ELISA, better neutralizing titers, better cell mediated immunity per ELIspot and intracellular cytokine staining, and improve protection in a live virus mouse model challenge experiment, grEc vaccines surface expressing modified and multimeric FPs inform further production of grEc vaccines for many Ags for infectious and other diseases, advancing development of a synthetic biology-based, inexpensive, vaccine approach suitable for global application and pandemic/biothreat rapid response, targeted cancer immunotherapy, and Ab production.

### IV. Research Strategy

Vaccines for global use and pandemic response, targeted cancer immunotherapy, and Ab production should be: 1) Easily modifiable to elicit immune responses against the targeted Ag, including new biothreats: 2) Very inexpensive: 3) Made using inexpensive, available feedstocks; and 4) Capable of being produced in existing factories around the world (Broadbent et al. 2011; Barret et al. 2009; De Groot et al. 2013). We developed such a platform: Killed Whole Cell (KWC) genome-reduced E. coli (grEc) that express vaccine antigens on their surfaces by means of a Gram-negative autotransporter (AT) (Maeda et al. 2021).

Gram-negative bacterial autotransporters (ATs). ATs, a protein family that enables bacteria to place proteins into the outer membrane (Jose & Meyer, 2007); van Bloois et al. (2011); Nicolay et al. (2015); Henderson et al. (2000), have 3 domains: an N-terminal signal sequence directing transport across the inner membrane that is subsequently cleaved: a C-terminal β-barrel that inserts into the outer membrane, yielding a pore-like structure; and a central passenger protein domain that transits through the pore. ‘displaying’ the passenger protein to the environment. Sequence encoding a protein of interest can replace native passenger protein sequence, yielding recombinant ATs that display ˜2×10⁵ proteins on each cell (reviewed in Jose et al. (2007); Nicolay et al. (2015)). ATs can express recombinant proteins up to ˜100 kDa (Jose et al. (2007); van Bloois et al. (2011)), making them useful for many potential vaccine applications. No high energy phosphate bonds are used in the AT-mediated pathway. Passenger protein export depends on the change in free energy in the extracellular presence of passenger protein. Hence, it should be possible to modify passenger protein export by altering the passenger protein amino acid residues (AAs).

A recombinant Gram-negative surface-expressed antigen killed whole cell (KWC) bacterial vaccine platform. KWC bacterial vaccines have a long, successful history, with currently licensed vaccines against diseases caused by the bacteria inactivated in the vaccine, e.g., cholera (Bi et al. (2017). We leveraged this long experience to create a new synthetic biology-based KWC bacterial vaccine platform that uses ATs to display new, recombinant, engineered vaccine Ags on the surfaces of genome-reduced E. coli (grEc). Using grEc vs. wt bacteria dramatically enhances the visibility of surface-expressed antigens (Maeda et al. 2021), grEc-platform KWC vaccines would be given PO or IN, inexpensive and scalable. For example 6 M doses of a WHO-prequalified oral cholera vaccine were produced in 1 y using a single 100 L fermenter at <$1/dose (Odevall et al. (2018)). 1500 L industrial fermenters are in use globally, including in low/middle income countries (LMICs), producing approved KWC vaccines, like cholera and pertussis vaccines. KWC E. coli vaccines have been extensively studied and are currently approved for agricultural use. (e.g., J-VAC) for bovine mastitis Holmgren & Bergquist, 2004; Hays et al., 2016; Evans et al., 1988; Savarino et al., 2009; Selim et al., 1995: Deluyker et al., 2005.

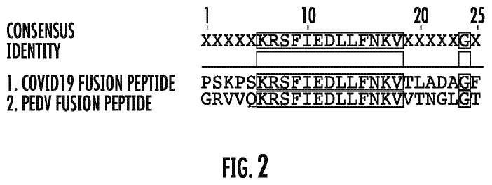

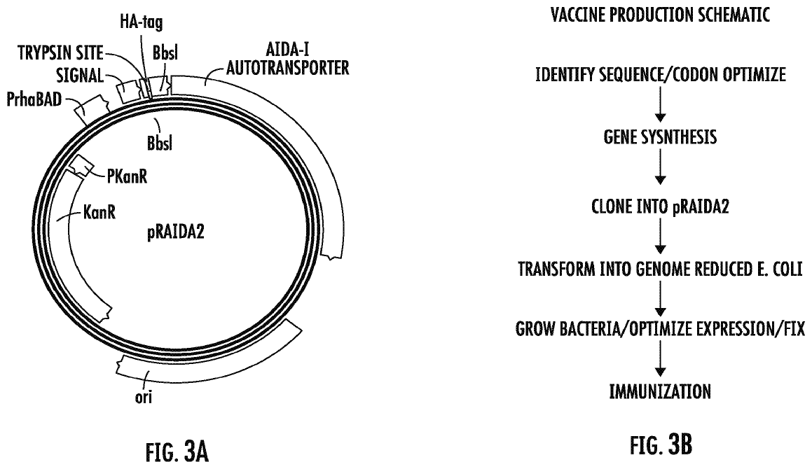

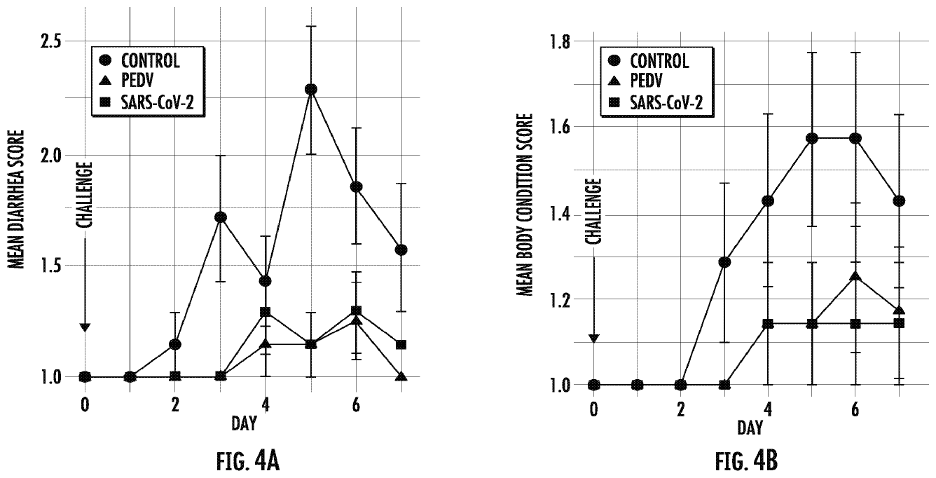

We used the platform to produce vaccines targeting the Fusion Peptide (FP) regions of the spike proteins of two different coronaviruses. SARS-CoV-2 and Porcine Epidemic Diarrhea Virus (FIG. 2). We made vaccines by cloning DNA encoding the FPs into our expression plasmid, pRAIDA2 (FIGS. 3A and 3B), transforming the construct into grEc (Kato & Hashimoto, 2007; Hashimoto et al., 2005), pRAIDA2 includes a Gram-autotransporter surface expression cassette that can place up to 2×10⁵ recombinant proteins on the surface of each bacterial cell, anchored into the bacteria's outer membrane by the beta-barrel domain of the autotransporter (Jose et al. (2007); van Ulsen et al. (2018); Meuskens et al. (2019)). We verified surface expression of FPs on grEc by flow cytometry and immunoblot-trypsinization experiments. We vaccinated pigs and challenged them with live PEDV (Maeda et al. 2021). We found that the vaccines effectively protected pigs against disease (FIGS. 4A and 4B) and that SARS-CoV-2 FP and PEDV FP vaccines were equally effective in preventing clinical disease, suggesting that a vaccine targeting the FP might represent a route to a universal coronavirus vaccine³. Recently, 3 groups described human anti-SARS-CoV-2 FP neutralizing monoclonal antibodies (Sun et al. (2022); Low et al. (2022); Dacon et al. (2022b)), validating FP as a vaccine target.

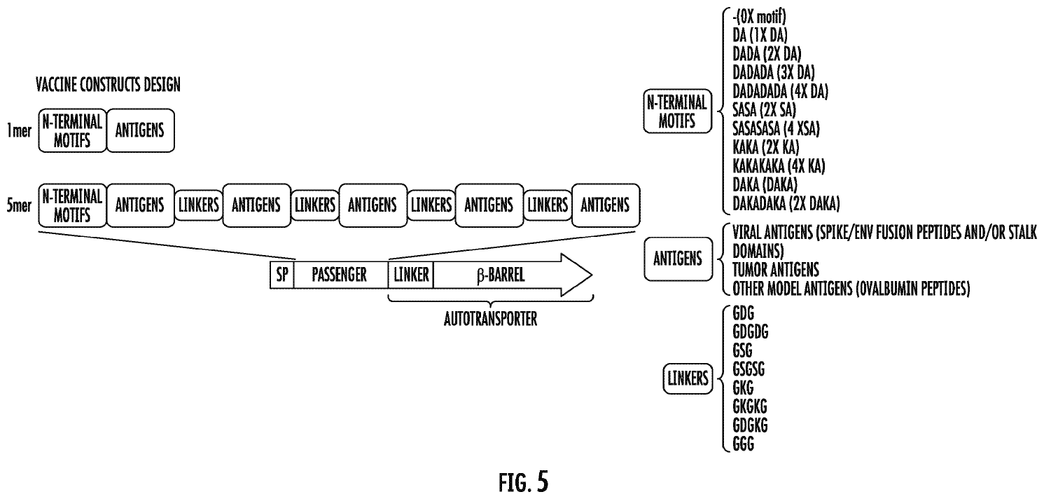

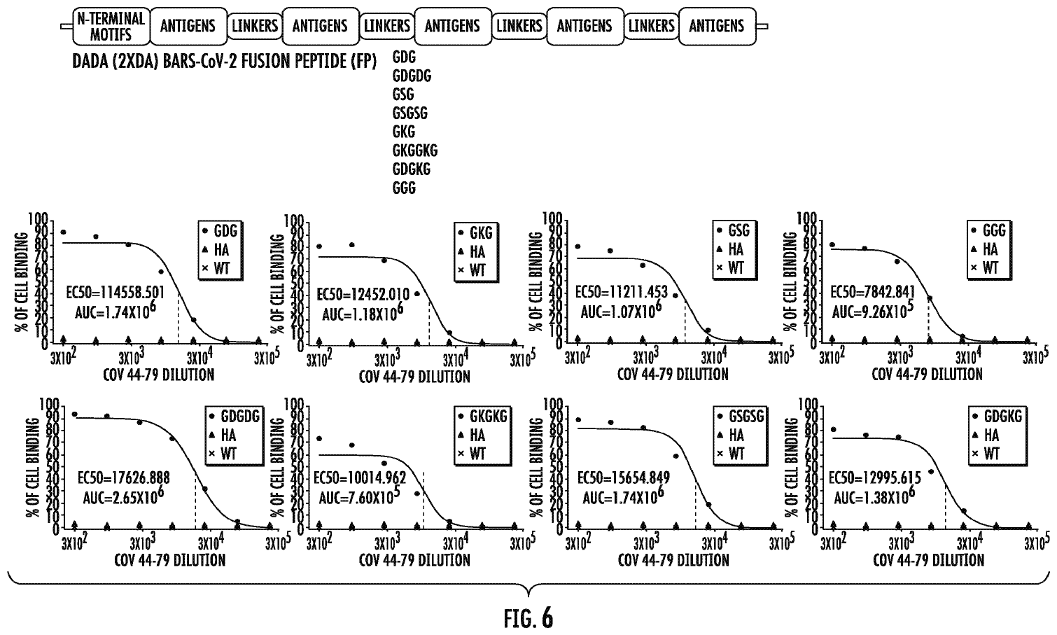

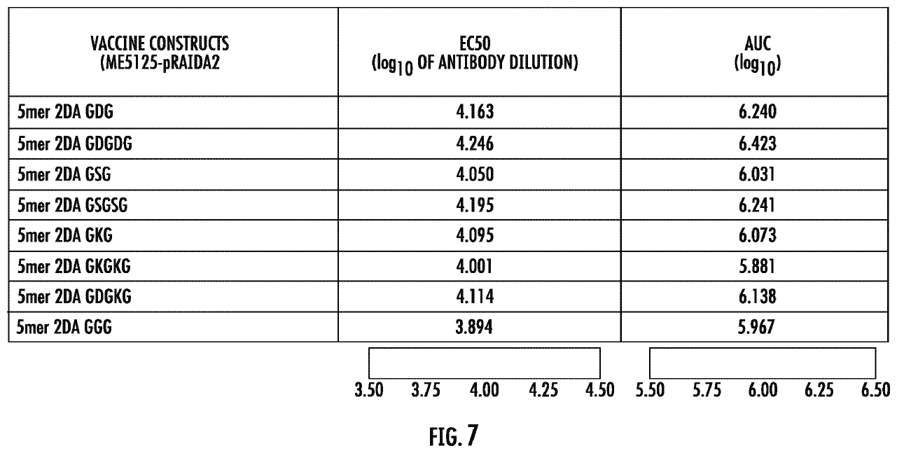

Beginning antigen optimization experiments. Without the resources from the grant, we could not maximally advance the project. Nevertheless, we began to design, construct, and test an initial series of modified 5-mer FP vaccines (FIG. 5), using COV 44-79, a human anti-FP BN MAb kindly provided via MTA by Josh Tan. NIAID (Dacon et al., 2022a). Results of testing a set of constructs, all with an added N-terminal asp-ala-asp-ala (DADA) (which we determined enhances expression and binding of AT surface-expressed Ags, not shown) and different linkers between the FP segments are shown in FIG. 6. The results are summarized in FIG. 7.

These results were obtained using a human anti-FP Broadly Neutralizing (BN) Monoclonal Antibody (MAb) kindly provided by Josh Tan. NIAID. The fact that our candidate FP vaccines effectively bind a BN MAb provides further evidence that they will likely elicit the desired neutralizing Ab response. The SARS-CoV-2 FP BN MAbs are highly analogous to HIV FP BN MAbs and suggests that our proposed vaccines will also elicit desired anti-FP Abs. The data further show that we can effectively evaluate and compare different constructs to select the ones that show the best binding to the MAb, which we will do with the HIV FP candidate vaccines. We will next compare the immunogenicity of the constructs with GDGDG (SEQ ID NO: 101). GSGSG (SEQ ID NO: 102), and GDGKG (SEQ ID NO: 103) to determine the best linker for vaccine use and use that knowledge to design our HIV FP constructs. In some embodiments, the fusion peptides of coronavirus and HIV are examples of proteins targeted by BN MAbs.

The fact that our candidate FP vaccines effectively bind a human BN MAb provides further evidence that they will likely elicit a desired Ab response. The data further show that we can effectively engineer, evaluate, and compare constructs to select the ones that show the best MAb binding. With the complete DBTL cycles we will be able to determine optimum configurations of Ags. N-terminal, and additional sequences.

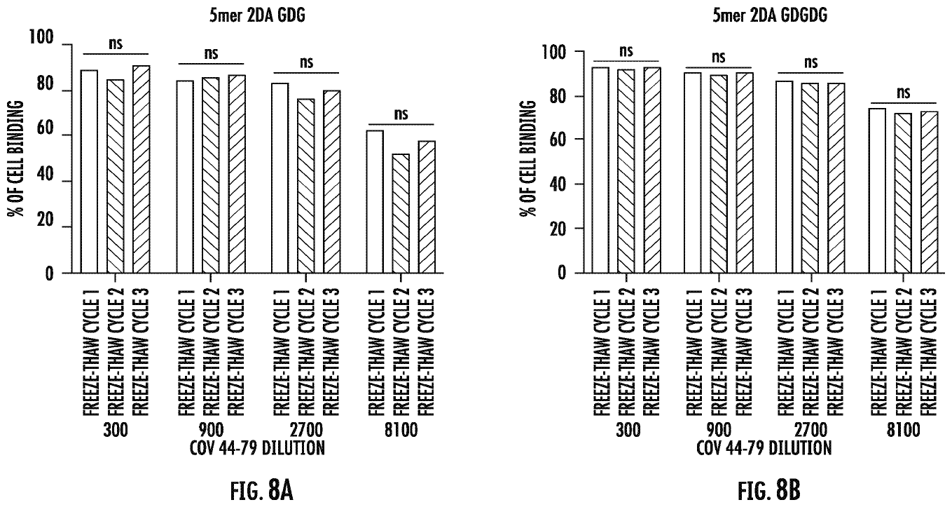

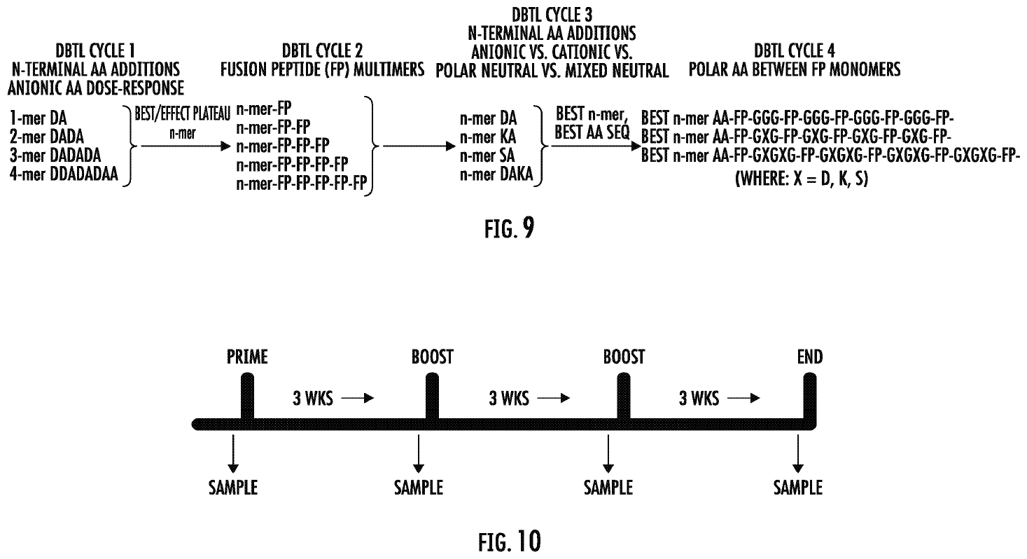

Because our vaccines are intended for global use, stability is an important design goal. We started stability testing and found that binding ability of the vaccines for COV 44-79 (Dacon et al., 2022a) is stable over 3 freeze-thaw cycles (FIGS. 8A and 8B), a desirable characteristic for the intended use case.

Overall, our additional preliminary data supports the proposition that we will be able to rapidly produce, test, and optimize KWC grEc vaccines, and that the vaccines are likely to have the desired stability characteristics, making it likely that we will be able to accomplish the goals of the proposed project. In some embodiments, the preliminary data supports the proposition that we will be able to rapidly produce, test, and optimize HIV Killed Whole Cell Genome-reduced E. coli vaccines for global use, and that the vaccines are likely to have the appropriate and desired stability characteristics, making it even more likely that we will be able to accomplish the goals of the proposed project.

Efforts have been directed at engineering enhanced surface expression of many recombinant passenger proteins, using discrete manipulations, like different signal sequences, but the focus has not been on enhancing production and/or display of vaccine Ags, particularly hydrophobic subunit Ags. Optimizations have generally not used a DBTL approach that leverage advances in synthetic biology. Hydrophobic Ags represent the most difficult expression case, since they may interact with the lipid bilayer of the bacterial outer membrane or aggregate with each other via hydrophobic interactions, which cause production of aggregates (Gorbenko G et al. (2011); Cromwell et al. (2006); Bolognesi B et al. (2013)). Approaches to enhance hydrophobic Ag expression and immunogenicity should also function for hydrophilic Ags.

Our preliminary data also illustrate the great strength of the platform in its ability to rapidly and inexpensively evaluate multiple different Ag modifications. This R21 application requests funds to conduct experiments to further optimize expression and assess the immunogenicity of Ags expressed using an AT in a KWC grEc platform. The data will significantly inform further development of the platform.

### Innovation

Our KWC grEc vaccine platform represents a new, promising solution for a globally appropriate, inexpensive, rapidly modifiable vaccine that can be readily produced in factories around the world. Our platform also has potential for cancer immunotherapy. It is particularly useful for expressing very hydrophobic antigens, but is also useful for other Ags. We use the SARS-CoV-2 FP because we have experience and baseline data in this system, the reagents and systems to assess immunogenicity, and the ability to elicit protective immunity in an animal model, constituting a useful test case. However, the technology we will develop in this project will be applicable to any protein antigen, and in that context, the project can be viewed as a general approach to producing better immune responses against any antigen made using the platform.

### V. DBTL Cycle 1. Enhancing Antigen Export to the Extracellular Space with Added Polar N-Terminal Amino Acids

Our preliminary experiments (not shown) demonstrated that adding DADA to the N-terminal of an FP 5-mer enhanced the amount of FP antigen produced. While this established the principle that we could enhance FP Ag production by adding N-terminal DAs, it did not establish an optimum number of added N-terminal hydrophilic AAs. In Cycle 1, we will conduct “dose-response” studies, comparing FP Ag production with 1-DA. 2-DA. 3-DA, and 4-DA. (4-DA is the limit for N-terminal DA additions, due to problems with repetitive sequences in Gibson-like assembly processes used by Twist). In cycle 1, we will determine optimal numbers of DAs for Ag expression. If we observe a plateau of effect, we will select the number of DAs at the plateau level.

### VI. DBTL Cycle 2. Enhancing Ag Dose by Expressing Ag Multimers

After we determine the optimal number of DA residues, we will compare expression and exposure of different FP multimers. In our preliminary experiments, we found that DADA-5-mer FP Ag was expressed better than the construct with no DAs. Because it may be that with more DAs. FP exposure will be greater, it will be important to rigorously test this. For example, it may be that 4- or 5-N-terminal DAs will be able to extend a 2- or 3-mer FP multimer away from the bacterial surface, but not a 5-mer FP. Therefore, in Cycle 2, we will compare expression by quantitative scanned immunoblot and exposure by flow cytometry of 1-, 2-, 3-, 4-, and 5-mer FPs with the optimum number of DAs determined in cycle 1.

### VII. DBTL Cycle 3. Comparing Anionic, Cationic, and Non-Charged N-Terminal Additions

Our preliminary data support the hypothesis that, for some constructs, adding hydrophilic DA amino acid residues to the passenger protein N-terminal will enhance expression. However, a thorough set of experiments is needed to determine what AAs will work best. We initially chose D because it is polar and because it is negatively charged. The bacterial surface has a net negative charge, so we hypothesized that additional Ds would also help push the Ag away from the bacterial surface by electrostatic repulsion. However, having a net negative N-terminal charge may not be favorable for subsequent Ag interactions with immune cells, like dendritic cells, since positively charged particles bearing vaccine antigens have enhanced dendritic cell interactions. Testing antigens with added positive and negatively engineered charged AAs, and with AAs with no added charge will be important³⁷. We may need to balance optimal Ag extension out away from the bacterial surface with engineering an Ag that optimally interacts with immune cells. Therefore, in Cycle 3, we will compare expression and exposure of the best FP multimer Ag determined in Cycle 2, with alternative N-terminal AAs: anionic DA, cationic KA, polar but non-charged SA, and alternating DAKA for a net neutral charge. If different N-terminal AAs yield similar expression we will select the neutral or positively charged version since those would likely better interact with the B-cell precursors.

### VIII. DBTL Cycle 4. Enhancing Ag Exposure in Ag Multimers by Adding Hydrophilic Amino Acid Residues Between Ag Monomers

We initially emphasize adding polar AA residues to the Ag N-terminal. However, directing modifications only toward the Ag N-terminal may not optimally extend multimeric FPs away from the cell surface. Hence, in cycle 4, we will take constructs identified as having the best expression and exposure in Cycles 1-3, and synthesize new versions that place GXG or GXGXG linkers (where X is D. K, or S) between the individual FP Ags in the multimer FPs. Our preliminary data (FIGS. 6 and 7) suggests that linkers with GDGDG (SEQ ID NO: 101). GSGSG (SEQ ID NO: 102), and GDGKG (SEQ ID NO: 103) will likely be best, but we will need to test these linkers combined with the other versions of the constructs, including different N-terminal modifications, to firmly establish the findings.

### IX. Producing, Comparing, and Conducting Quality Control (QC) on KWC Modified/Multimerized FP grEc Vaccines

We will produce sufficient quantities of hypothesized enhanced immunogenicity vaccines for mod 1, mod 2, and mod 3, and the prototype unmodified 1-mer and positive (HA immunotag) and negative (bacteria not transformed with an expression plasmid) controls using quantitative immunoblots and antibody binding in flow cytometry to QC each vaccine lot³. We will measure endotoxin levels in vaccine lots (ThermoFisher), although endotoxin-related toxicity was not a problem in our previous study (Maeda et al. (2021)). Endotoxin should not be an issue for non-parenteral routes. For the IM route, if the mice exhibit significant endotoxin-related toxicity in pilot injections we will deplete endotoxin using endotoxin bead adsorption. Licensed, safe and effective, albeit sometimes reactogenic KWC pertussis IM vaccines have ˜5×10⁵ IU/mg protein (Dias et al. (2013)), which will be our benchmark.

### X. Comparing Immunogenicity of KWC Modified/Multimerized FP grEc Vaccines

To test the ability of the modified vaccines to elicit effective immune responses, we will prepare vaccines and vaccinate mice (C₅₇BL [6 w old]-background strain for K18-hACE2 used in the protection studies). After acclimation, we will obtain a pre-bleed, then vaccinate with 10⁸ or 10⁹ cells. Vaccination routes tested will include IM. IN, and PO (by gavage, with NaHCO₃ to buffer gastric acidity and decrease gastric transit time) with mod 1, mod 2, and mod 3, since different versions may exhibit different immunogenicity effects depending on administration route.

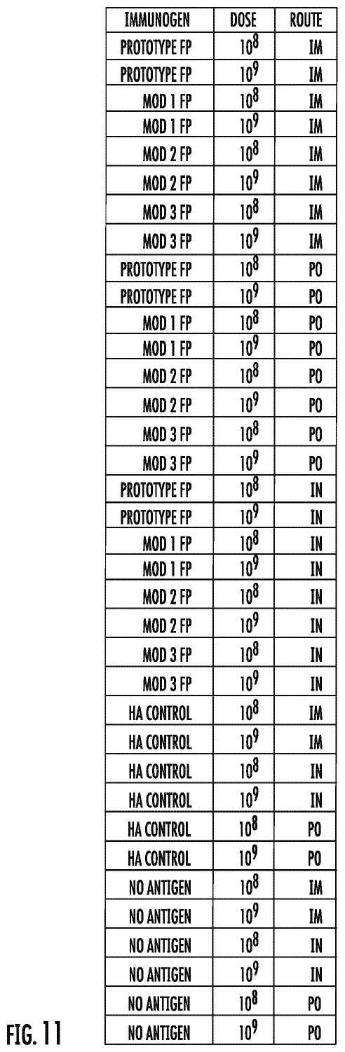

We will boost at 3 weeks and 6 weeks after prime, collecting sera 5d before boosts, then end the study 3 weeks after final boost, with collection of terminal bleed samples and spleen cells for the ELIspot and CD4 and CD8 T cell response assays (below). See FIGS. 10 and 11. We chose this schedule because it agrees with well-established schedules and because in our previous study we observed good anamnestic responses after a prime plus 1 boost, suggesting that a prime plus 2 boost schedule will likely improve responses. A 50 μl PBS vaginal wash and a nasopharyngeal wash will be obtained with each blood collection to enable assessment of mucosal humoral immunity. Vaginal washes have been used as a minimally invasive way of evaluating mucosal immune responses from a variety of vaccination routes (Lebre F et al. (2016); Neutra et al. (2006); Pavot et al. (2012); Holmgren et al. (2005)).

ELISAs to detect vaccine antigen-specific antibody response, We will determine anti-SARS-CoV-2-FP antibodies in sera/washes by ELISA. The ELISA will be a minor modification of previously published procedures (He et al. (2004)). We will coat 96-well plates with synthetic fusion peptide [SFIEDLLFNKVTLADAGF: SEQ ID NO: 3]. We will measure bound antibodies with HRP-conjugated anti-human antibodies (Invitrogen), developing plates with TMB. Positive controls will include anti-FP rabbit antisera and the COV 44-79 human BN anti-FP MAb (Dacon et al., 2022a).

ELISpot assays. We will perform ELISpot assays (Slota et al. (2011)) per manufacturer's instructions (Mabtech). We will plate spleen cells (5×10⁶ cells/mL), stimulated in the presence or not of synthetic FP peptide [SFIEDLLFNKVTLADAGF: SEQ ID NO: 3] that is expressed in the vaccines, using a biotinylated detection antibody. Positive spots will be identified with Streptavidin-ALP followed by substrate solution (BCIP/NBT-plus).

CD4 and CD8 T cell response, We will assay for FP-specific T cell responses by intracellular cytokine staining. We will stimulate 1×10⁶ splenocytes seeded in 96-well plates with Cell Stimulation Cocktail (ThermoFisher), mouse IL-2 and FP peptide, in PHA (positive control), or medium only (mock) parallel assays. After stimulation, we will stain cells for surface markers APC-H7, rat anti-mouse CD4 and PE Rat anti-mouse CD8a (BD), staining intracellularly with Alexa Fluor 488 Rat anti-mouse IFN-γ (BD). PerCp/Cy5.5 Rat anti-mouse IL-2, BV421 Rat anti-mouse IL-4 BD, BV605 Rat anti-mouse TNF-α and APC Rat anti-mouse CD107a, and cytokine bead array (Weber et al. (2013a), Weber et al. (2013b)).

We will examine cells by flow cytometry using an LSRFortessa, and analyze with FlowJo V software. We will examine frequencies of polyfunctional CD4+ T cells (IFN-γ and IL-2 [Th1]) and poly-functional CD8+ T cells producing both IFN-γ and TNF-α among stimulated CD3+ T cells. Evaluation of sera wash neutralization activity. We will compare the ability of the vaccines to elicit immune responses against FP by comparing the ELISA titers, neutralizing titers using a modified plaque reduction assay (Baer (2014)), all for sera and wash samples from each sampling point during the study and at the end of the study, and by ELIspot assays using cells obtained at the end of the experiment. For the neutralization assays will grow SARS-CoV-2 strain WA-1 (BEI Resources) on Vero E6 cells in DMEM 10% FBS. We will titer virus in triplicate using Vero E6 indicator cells in a 1.5% Avicel (methyl cellulose) growth medium, and store at −80° C., at a titer of 10⁶ pfu/ml for use within 90 days. For the assay, we will incubate virus at 30 pfu with serial 5-fold dilutions of sera or wash, incubate at 37° C. for 30′, and place in triplicate confluent Vero E6 cells in a 12-well plate; positive neutralization (Acros SAD-35 anti-RBD antibody) and negative controls consist of 30 PFU diluted in growth medium. Plates are incubated at 37° C. in 5% CO₂. Avicel is added and cells are incubated for 72 h, followed by formalin fixation and staining with crystal violet, followed by plaque counting. The primary selection criteria for the vaccine to be used in the challenge experiments will be the ability to elicit neutralizing Ab, the secondary criteria will be the ability to elicit anti-FP Ab per the ELISA results: tertiary criteria will be the ability to elicit CMI using the ELIspot results.

### XI. Comparison of Modified KWC grEc Vaccines to Protect Against Challenge Infection

While testing immune responses elicited by the modified KWC grEc FP vaccines will provide an initial evaluation, the ultimate evaluation of the vaccines will depend upon their ability to prevent disease. This may depend upon the effectiveness of the vaccines in eliciting both humoral and CMI responses and in the ability of the vaccinated animals to use both immune system arms to prevent disease. Non-neutralizing Ab may modify via antibody-dependent cytotoxic (virion-toxic) immunity, and it is plausible that some vaccines may be better or worse at accomplishing this. To study vaccines ability to protect against disease, we will use a modification of the K18-hACE2 model (McCray et al. (2007); Sun et al. (2020); Oladunni et al. (2020)). We will vaccinate treatment groups of 24 mice, using the schedule described in Aim 2.2, selecting the most immunogenic modified grEc KWC vaccine/dose/route using the above criteria. Controls will consist of the prototypical 1-mer FP KWC from grEc, and negative controls including KWC grEc expressing HA or not expressing any antigen. At 21 dp boost 2, mice will be each challenged intranasally with a 1250 PFU of SARS-CoV-2, initially Wuhan, but later we will assess protection against variants, selecting variant(s) most prevalent at the time. We will monitor body weight of infected mice daily and score the following as clinical signs of disease: activity, respiratory effort, and body condition. At 3- and 7-days post-challenge (DPC), 8 mice from each group will be necropsied to collect bronchoalveolar lavage (BAL) fluid, lung, splenocytes, and blood samples. At necropsy, the single left lobe will be ligated prior to BAL, with the tie removed before fixation, with only the left lung sent for histology, to preserve histologic integrity. Neutralizing antibody titers in BAL fluid and serum will be determined. We will determine post-challenge T-cell profile status using isolated splenocytes collected at necropsy. Viral loads in BAL fluid and lung sample will be determined by RT-qPCR and plaque assay. Gross lesions in lung tissues will be examined and scored by a board-certified veterinary pathologist. At necropsy, we will collect samples of lung tissues from each mouse for quantification of viral loads and proinflammatory cytokines and chemokines by Luminex microbead assay through the UVA Flow Core (Mouse 32-plex). We will compare the survival rate, lung/BAL virus load, gross and histological lesions, and pre- and post-challenge virus-specific immune responses between vaccinated and unvaccinated groups.

Reproducibility and Experimental Design Considerations. We will use specific pathogen-free, female mice (to enable evaluation of mucosal immunity via vaginal washes. Jackson Labs). The

$$n \geq {2 [ {z}_{α / 2} + {z}_{β} ]}^{2} ( \frac{{σ}_{pre , study}^{2}}{{δ}_{pre , study}} )$$

estimated sample size in the animal study to obtain >0.9 power is n≥7 based on a power analysis using an ANOVA or ANCOVA model. The formula:
informs the sample size (n), where σ2 is representative of typical immunization studies: typical variance of error, δ. The difference between 2 treatment groups, a: the significance level of the test, or the probability of Type I error, i.e., α=0.05. β: probability of a Type II error. The power of test is 1-β. Thus. 8 animals per group are sufficient to produce statistically meaningful data. Significance will be assessed at P<0.05. We will conduct tests for normality prior to conducting the above analysis, and a non-parametric test (Kruskal-Wallis) will be used if needed. All analyses will be performed using R, in the Rstudio environment, with included packages, and with tidyverse and stats packages, with visualization using ggplot2.

Expected outcomes. We expect to produce a modified/multimerized FP KWC grEc vaccine. We expect that a modified/multimerized KWC FP vaccine will elicit sera with a significantly higher FP antigen-specific binding by ELISA, and better neutralizing and T-cell immune responses against SARS-CoV-2, and an enhanced protection against disease following experimental infection.

### REFERENCES

All references listed in the instant disclosure, including but not limited to all patents, patent applications and publications thereof, scientific journal articles, and database entries, are incorporated herein by reference in their entireties to the extent that they supplement, explain, provide a background for, and/or teach methodology, techniques, and/or compositions employed herein. The discussion of the references is intended merely to summarize the assertions made by their authors. No admission is made that any reference (or a portion of any reference) is relevant prior art. Applicants reserve the right to challenge the accuracy and pertinence of any cited reference.
Altschul et al. (1990a) J Mol Biol 215:403-410.
Altschul et al. (1990b) Proc Natl Acad Sci USA 87:14:5509-13.
Altschul et al. (1997) Nucleic Acids Res 25:3389-3402.
Baer (2014). J Vis Exp. November 4(93)
Barrett A. Stanberry L. Academic Press: 2009.
Berry et al. (2003) Hybrid Hybridomics 22(1):23-31.
Bi et al. (2017) Lancet Infect Dis 17:1080-1088.
Bodanszky & Bodanszky (1984) in The Practice of Peptide Synthesis. Springer-Verlag. New York. New York. United States of America.
Bolognesi B et al. (2013) Prog Mol Biol Transl Sci. 117:53-72.
Broadbent et al. Curr Opin Virol. October; 1(4):254-62
Chen et al. (2010) Proc Natl Acad Sci USA 107(7):3099-104. PMC2840271
Chou & Fasman (1974) Biochemistry 13:222-245.
Chou & Fasman (1978) Ann Rev Biochem 47:251-276.
Chou & Fasman (1979) Biophys J 26:367-384.
Colligan et al. (eds.) (1994-2004) Current Protocols in Immunology, John Wiley & Sons, Inc., New York, New York, United States of America.
Cromwell et al. (2006) Aaps j. September 15; 8(3):E572-9.
Dacon et al. (2022a) Science. August 12; 377(6607):728-35
Dacon et al. (2022b) bioRxiv. Apr. 12, 2022. 04.11
Datsenko & Wanner (2000) Proc Natl Acad Sci USA 97:6640-6645.
Davis (2001) Adv Drug Deliv Rev. 51(1-3):21-42.
De Groot et al. (2013) Hum Vaccin Immunother. September: 9(9):1877-84.
Deluyker et al. (2005) Wageningen p. 285-9.
Deutscher et al. (ed.) (1990) Guide to Protein Purification, Harcourt Brace Jovanovich, San Diego. California. United States of America.
Devereux et al. (1984) Nucl Acids Res 12:387.
Dias et al. (2013) Hum Vaccin Immunother. February; 9(2):339-48.
Evans et al. (1988) FEMS Microbiol Immunol. December; 1(3):117-25.
Gerdes et al. (1984) J Immunol. 133(4):1710-5.
Gerhardt et al. (eds.) (1994) Methods for General and Molecular Bacteriology, American Society for Microbiology. Washington, DC. United States of America. page 574.
Gorbenko G et al. (2011) Adv Protein Chem Struct Biol. 84:113-42.
Graham et al. (2019) Annu Rev Med. 2019 Jan. 27; 70:91-104.
Greenfield (ed.) (2013) Antibodies: A Laboratory Manual. 2nd Ed. Cold Spring Harbor Laboratory Press. Cold Spring Harbor, New York. United States of America.
Gross & Mienhofer (eds.) (1981) The Peptides, Vol. 3, Academic Press, New York, New York. United States of America. pages 3-88.
Harlow et al. (1988) Cold Spring Harbor Laboratory
Hashimoto et al. (2005) Mol Microbiol 55:137-149.
Hays et al. (2016) BMC Res Notes. August 11; 9(1):401.
He et al. (2004) J Immunol. 2004 Sep. 15; 173(6):4050-7.
Henderson et al. (2000) Trends Microbiol 8(12):529-32.
Holmgren & Bergquist (2004) in New Generation Vaccines. Third Edition. Levine et al. (eds.) Marcel
Dekker. Inc., New York, New York, United States of America. Pages 991-1014.
Holmgren et al. (2005) Nature Medicine. 11(4):S45-S53.
Huang et al. (2012) Nature 491(7424):406-12. PMC4854285
Jabbal-Gill (2010) J Drug Target. 18(10):771-86.
Jose & Meyer (2007) Microbiol Mol Biol Rev 71(4):600-619.
Karlin & Altschul (1990) Proc Natl Acad Sci USA 87:2264-2268.
Karlin & Altschul (1993) Proc Natl Acad Sci USA 90:5873-5877.
Kato & Hashimoto (2007) Mol Syst Biol. 3:132.
Kelly et al. (2019) Expert Review of Vaccines. 2019; 18(3):269-80.
Kilpatrick et al. (1997) Hybridoma 16(4):381-9.
Kittlesen et al. (1998) J Immunol 60:2099-2106.
Klebanoff et al. (2005) Proc Natl Acad Sci USA. 102(27):9571-6.1172264
Klebanoff et al. (2006) Immunol Rev 211:214-24. PMC1501075
Kyte & Doolittle (1982) J Mol Biol 157:105-132.
Lebre F et al. (2016) Molecular Pharmaceutics. 13(2):472-82.
Li et al. (2008) Biotechnol Bioeng 99(2):485-90.
Low et al. (2022). Science 377(6607):735-42.
Maecker et al. (2005) BMC Immunol 6:17. PMC1190174
Maecker et al. (2012) Nat Rev Immunol. 12(3):191-200. PMC3409649
Maeda et al. (2021) Killed whole-genome reduced-bacteria surface-expressed coronavirus fusion peptide vaccines protect against disease in a porcine model. PNAS 118(18):e2025622118.
McCray et al. (2007) J Virol. January; 81(2):813-21
Meuskens et al. (2019) Front Microbiol 10:1163.
Morabito et al. (2017) Mucosal Immunol. 10(2):545-54. PMC5123975
Morgan & Gainor (1989) Ann Rep Med Chem 24:243-252.
Neutra et al. (2006) Nat Rev Immunol. February; 6(2):148-58.
Nicolay et al. (2015) Crit Rev Microbiol 41(1):109-23.
Nizard et al. (2017) Nat Commun 8:15221. PMC5458068
Odevall et al. (2018) Vaccine 36:6606-6614.
Oladunni et al. (2020) Nat Commun. November 30; 11(1):6122
Oliveira et al. (2007) Expert Rev Vaccines 6(3):419-29.
Pavot et al. (2012) Vaccine. January 5; 30(2):142-54.
PCT International Patent Application Publication Nos. WO 1999/54464: WO 2004/110486: WO 2006/012539; WO 2020/046982: WO 2021/076897.
PCT International Patent Application Serial No. PCT/US2005/026102.
Rapp & Kaufmann (2004) Int Immunol 16:597-605.
Riese et al. (2014) Expert Opin Drug Deliv 11(10):1619-34.
Russell (2002) Am J Reprod Immunol 47(5):265-8.
Savarino et al. (2009). Retrovirology. 6:52.2697151
Seder & Ahmed (2003) Nat Immunol 4(9):835-42.
Selim et al. (1995) Vaccine. March; 13(4):381-90.
Shata et al. (2000) Mol Med Today 6 (2):66-71.
Shoemaker et al. (2005) Infect Immun 73(8):5031-8. PMC1201206
Slota et al. (2011) Expert Rev Vaccines. March; 10(3):299-306.
Spatola (1983) Chemistry and Biochemistry of Amino Acids, Peptides and Proteins, Vol. VII. Weinstein (ed.). Marcel Dekker, Inc., New York, New York. United States of America, pages 267-357.
Stewart et al. (1984) in Solid Phase Peptide Synthesis, 2nd Edition, Pierce Chemical Company, Rockford, Illinois, United States of America.
Sun et al. (2020) Cell Host Microbe. 2020 May 27
Sun et al. (2022) Nature Microbiology 7(7):1063-74.
Tam et al. (2016) Proc Natl Acad Sci USA. 113(43):E6639-E48. PMC5086995
U.S. Patent Application Publication No. 2008/0193469.
U.S. patent application Ser. No. 11/572,453.
U.S. Pat. Nos. 4,554,101; 8,012,932.
Ulmer et al. (2006) Nat Biotechnol 24(11):1377-83.
van Bloois et al. (2011) Trends Biotechnol. 29(2):79-86.
van Ulsen et al. (2018) FEMS Microbiol Letters. 365(18)
Weber et al. (2013a) Clin Cancer Res. September 15; 19(18):5079-91.
Weber et al. (2013b). Leukemia. 2013 July; 27(7):1538-47
Welters et al. (2004) Vaccine 23:305-311.
Yusuf & Kett (2017) Hum Vaccin Immunother. 13(1):34-45. PMC5287317
Zaman et al. (2013) Drug Deliv Transl Res. 2013; 3(1):100-9. PMC3539070
Zugel (2001) Infect Immun 69:4164-4167.

It will be understood that various details of the presently disclosed subject matter can be changed without departing from the scope of the presently disclosed subject matter. Furthermore, the foregoing description is for the purpose of illustration only, and not for the purpose of limitation.

1. An expression cassette comprising a first coding sequence encoding at least one polar amino acid or at least one expression enhancement peptide comprising one or more polar amino acids, a second coding sequence encoding a polypeptide of interest, and a third coding sequence encoding a bacterial autotransporter β-barrel polypeptide, wherein:
(i) the first, second, and third coding sequences are in frame with each other such that transcription and translation of the expression cassette in a host cell produces a fusion protein comprising the polar amino acid and/or the expression enhancement peptide, the polypeptide of interest, and the bacterial autotransporter β-barrel polypeptide;
(ii) the expression enhancement peptide comprises one or more copies of the amino acid sequence (X¹X²X³)ₙ, wherein each X¹, X², X³ is independently selected from the group consisting of Asp (D), Asn (N), Glu (E), Gln (Q), His (H), Arg (R), Lys (K); Ala (A), Ser(S), Thr (T), Pro (P), Gly (G), Met (M), Leu (L), Ile (I), Val (V), and Cys (C); each X³ is absent in any given member of the one or more copies of the amino acid sequence (X¹X²X³)ₙ or, if present, is independently any amino acid; and n is at least 1; and
(iii) when expressed in a host cell, optionally a bacterium, further optionally a Gram-negative bacterium, the expression cassette induces expression of the polypeptide of interest on a surface of the host cell.

2. The expression cassette of claim 1, wherein the polypeptide of interest is a concatemer of two, three, four, five, or more copies of an amino acid sequence of interest, optionally wherein each of the two, three, four, five, or more copies of the amino acid sequence of interest are linked to each other via a peptide linker, and further optionally wherein the peptide linker comprises a peptide sequence selected from the group consisting of FGGG (SEQ ID NO: 87), GGGF (SEQ ID NO: 88), SGGG (SEQ ID NO: 89), GGGS (SEQ ID NO: 90), and any combination thereof.

3. (canceled)

4. The expression cassette of claim 2, wherein at least one of the two or more copies of the amino acid sequence of interest in the concatemer comprises an expression enhancement peptide at its N-terminus, optionally wherein each of the two or more copies of the amino acid sequence of interest in the concatemer comprises an expression enhancement peptide at its N-terminus.

5. The expression cassette of claim 1, wherein the expression enhancement peptide comprises an amino acid sequence selected from the group consisting of DA, DS, DT, DP, DG, NA, NS, NT, NP, NG, EA, ES, ET, EP, QA, QG, QA, QS, QT, QP, QG, HA, HS, HT, HP, HG, RA, RS, RT, RP, RG, LA, LS, LT, LP, LG, DAX⁴, DSX⁴, DTX⁴, DPX⁴, DGX⁴, NAX⁴, NSX⁴, NTX⁴, NPX⁴, NGX⁴, EAX⁴, ESX⁴, ETX⁴, EPX⁴, QAX⁴, QGX⁴, QAX⁴, QSX⁴, QTX⁴, QPX⁴, QGX⁴, HAX⁴, HSX⁴, HTX⁴, HPX⁴, HGX⁴, RAX⁴, RSX⁴, RTX⁴, RPX⁴, RGX⁴, LAX⁴, LSX⁴, LTX⁴, LPX⁴, LGX⁴, DADA (SEQ ID NO: 11), EAEA (SEQ ID NO: 12), DAEA (SEQ ID NO: 13), EADA (SEQ ID NO: 14), NANA (SEQ ID NO: 15), QAQA (SEQ ID NO: 16), DANA (SEQ ID NO: 17), EANA (SEQ ID NO: 18), NADA (SEQ ID NO: 19), NAQA (SEQ ID NO: 20), HAHA (SEQ ID NO: 21), RARA (SEQ ID NO: 22), LALA (SEQ ID NO: 23), HSHS (SEQ ID NO: 24), HTHT (SEQ ID NO: 25), LSLS (SEQ ID NO: 26), LTLT (SEQ ID NO: 27), RSRS (SEQ ID NO: 28), RTRT (SEQ ID NO: 29), HPHP (SEQ ID NO: 30), HGHG (SEQ ID NO: 31), LPLP (SEQ ID NO: 32), LGLG (SEQ ID NO: 33), DSDS (SEQ ID NO: 34), ESES (SEQ ID NO: 35), DSES (SEQ ID NO: 36), ESDS (SEQ ID NO: 37), NSNS (SEQ ID NO: 38), QSQS (SEQ ID NO: 39), DSNS (SEQ ID NO: 40), ESNS (SEQ ID NO: 41), NSDS (SEQ ID NO: 42), NSQS (SEQ ID NO: 43), DTDT (SEQ ID NO: 44), ETET (SEQ ID NO: 45), DTET (SEQ ID NO: 46), ETDT (SEQ ID NO: 47), NTNT (SEQ ID NO: 48), QTQT (SEQ ID NO: 49), DTNT (SEQ ID NO: 50), ETNT (SEQ ID NO: 51), NTDT (SEQ ID NO: 52), NTQT (SEQ ID NO: 53), DPDP (SEQ ID NO: 54), EPEP (SEQ ID NO: 55), DPEP (SEQ ID NO: 56), EPDP (SEQ ID NO: 57), NPNP (SEQ ID NO: 58), QPQP (SEQ ID NO: 59), DPNP (SEQ ID NO: 60), EPNP (SEQ ID NO: 61), NPDP (SEQ ID NO: 62), NPQP (SEQ ID NO: 63), DGDG (SEQ ID NO: 64), EGEG (SEQ ID NO: 65), DGEG (SEQ ID NO: 66), EGDG (SEQ ID NO: 67), NGNG (SEQ ID NO: 68), QGQG (SEQ ID NO: 69), DGNG (SEQ ID NO: 70), EGNG (SEQ ID NO: 71), NGDG (SEQ ID NO: 72), NGQG (SEQ ID NO: 73), HAHS (SEQ ID NO: 74), RARS (SEQ ID NO: 75), LALS (SEQ ID NO: 76), HSHT (SEQ ID NO: 77), HTHP (SEQ ID NO: 78), LSLT (SEQ ID NO: 79), LTLP (SEQ ID NO: 80), RSRT (SEQ ID NO: 81), RTRP (SEQ ID NO: 82), HPHG (SEQ ID NO: 83), HGHA (SEQ ID NO: 84), LPLA (SEQ ID NO: 85), LGLA (SEQ ID NO: 86), and combinations thereof, wherein X⁴ is any amino acid.

6. The expression cassette of claim 1, wherein the polypeptide of interest is an antigen, optionally an antigen selected from the group consisting of an antigen from a cancer cell, an antigen from a molecule involved in an autoimmune response, and an antigen from a pathogen, further optionally a viral antigen, and further optionally wherein the viral antigen comprises a coronavirus antigen, optionally a severe acute respiratory syndrome-associated coronavirus (SARS-CoV) antigen, a severe acute respiratory syndrome-associated coronavirus 2 (SARS-CoV-2) antigen, and/or a porcine epidemic diarrhea virus (PEDV) antigen; or a human immunodeficiency virus (HIV) antigen.

7. (canceled)

8. The expression cassette of claim 6, wherein the viral antigen comprises:
(i) a coronavirus fusion peptide (FP) antigen, optionally a coronavirus FP antigen comprising an amino acid sequence selected from the group consisting of PSKPSKRSFIEDLLFNKVTLADAGF (SEQ ID NO: 2), PSKPSKRSFIEDLLFNKVTLADAG (SEQ ID NO: 1), SFIEDLLF (SEQ ID NO: 4), SFIEDLLFNKVTLADAGF (SEQ ID NO: 3), GRVVQKRSFIEDLLFNKVVTNGLG (SEQ ID NO: 5), and immunogenic fragments thereof; or
(ii) an HIV antigen comprising an amino acid sequence selected from the group consisting of AVGIGAVF (SEQ ID NO: 7), ALGIGAAF (SEQ ID NO: 8), AVGFGAAF (SEQ ID NO: 9), AAGFGAMF (SEQ ID NO: 10), and immunogenic fragments thereof; or
(iii) any combination thereof.

9. The expression cassette of claim 1, wherein the expression vector is configured to express the polypeptide of interest in a genome reduced bacterium or derivative thereof, optionally on a surface of the genome reduced bacterium or derivative thereof.

10. The expression cassette of claim 1, wherein one or more of the first, second, and third coding sequences comprises a codon optimized coding sequence.

11. An expression vector comprising the expression cassette of claim 1, optionally wherein the expression cassette is operably linked to an inducible promoter or a constitutive promoter.

12-19. (canceled)

20. A vaccine composition comprising the expression cassette of claim 1 and a pharmaceutically acceptable carrier, diluent, or excipient, one or more adjuvants, or any combination thereof.

21. The vaccine composition of claim 20, wherein the vaccine composition comprises a bacterium, optionally a genome reduced bacterium, and further wherein the bacterium is a live attenuated bacterium or a killed whole cell bacterium or an immunogenic fragment thereof that comprises the polypeptide of interest.

22. The vaccine composition of claim 20, wherein the vaccine composition is formulated to be administered orally, rectally, vaginally, intra-nasally, parenterally, intradermally, subcutaneously, or intramuscularly.

23. (canceled)

24. The vaccine composition of claim 20, wherein the polypeptide of interest is an antigen, optionally a viral antigen, further optionally wherein the viral antigen comprises a coronavirus antigen, optionally a severe acute respiratory syndrome-associated coronavirus (SARS-CoV) antigen, a severe acute respiratory syndrome-associated coronavirus 2 (SARS-CoV-2) antigen, and/or a porcine epidemic diarrhea virus (PEDV) antigen; or a human immunodeficiency virus (HIV) antigen.

25. (canceled)

26. The vaccine composition of claim 24, wherein the viral antigen comprises:
(i) a coronavirus fusion peptide (FP) antigen, optionally a coronavirus FP antigen comprising an amino acid sequence selected from the group consisting of PSKPSKRSFIEDLLFNKVTLADAGF (SEQ ID NO: 2), PSKPSKRSFIEDLLFNKVTLADAG (SEQ ID NO: 1), SFIEDLLF (SEQ ID NO: 4), SFIEDLLFNKVTLADAGF (SEQ ID NO: 3), GRVVQKRSFIEDLLFNKVVINGLG (SEQ ID NO: 5), and immunogenic fragments thereof; or
(ii) an HIV antigen comprising an amino acid sequence selected from the group consisting of AVGIGAVF (SEQ ID NO: 7), ALGIGAAF (SEQ ID NO: 8), AVGFGAAF (SEQ ID NO: 9), AAGFGAMF (SEQ ID NO: 10), and immunogenic fragments thereof; or
(iii) any combination thereof.

27. A method for inducing an immune response in a subject, the method comprising administering to the subject a composition comprising, consisting essentially of, or consisting of the expression cassette of claim 1 in an amount and via a route sufficient to induce an immune response to the polypeptide of interest in the subject, optionally wherein the composition is administered parenterally.

28. The method of claim 27, wherein the immune response to the polypeptide of interest induced in the subject is enhanced relative to an immune response that would be induced in the subject when an expression cassette that is identical to the expression cassette of claim 1 is administered to the subject in the same amount and via the same route.

29-30. (canceled)

31. A method for enhancing expression of a polypeptide of interest in a host cell, the method comprising modifying the polypeptide of interest to include at least one N-terminal polar amino acid and/or an expression enhancement peptide moiety at or near its N-terminus, wherein the expression enhancement peptide comprises one or more copies of the amino acid sequence (X¹X²X³)ₙ, wherein each X¹ is independently selected from the group consisting of Asp (D), Asn (N), Glu (E), Gln (Q), His (H), Arg (R), and Lys (K); each X² is independently selected from the group consisting of Ala (A), Ser(S), Thr (T), Pro (P), Gly (G), Met (M), Leu (L), Ile (I), Val (V), and Cys (C); each X³ is absent in any given member of the one or more copies of the amino acid sequence (X¹X²X³)ₙ or, if present, is independently any amino acid; and n is at least 1, optionally wherein the expression enhancement peptide moiety comprises an amino acid sequence selected from the group consisting of DA, DS, DT, DP, DG, NA, NS, NT, NP, NG, EA, ES, ET, EP, QA, QG, QA, QS, QT, QP, OG, HA, HS, HT, HP, HG, RA, RS, RT, RP RG, LA, LS, LT, LP, LG, DAX⁴, DSX⁴, DTX⁴, DPX⁴, DGX⁴, NAX⁴, NSX⁴, NTX⁴, NPX⁴, NGX⁴, EAX⁴, ESX⁴, ETX⁴, EPX⁴, QAX⁴, OGX⁴, QAX⁴, OSX⁴, QTX⁴, QPX⁴, QGX⁴, HAX⁴, HSX⁴, HTX⁴, HPX⁴, HGX⁴, RAX⁴, RSX⁴, RTX⁴, RPX⁴, RGX⁴, LAX⁴, LSX⁴, LTX⁴, LPX⁴, LGX⁴, DADA (SEQ ID NO: 11), EAEA (SEQ ID NO: 12), DAEA (SEQ ID NO: 13), EADA (SEQ ID NO: 14), NANA (SEQ ID NO: 15), QAQA (SEQ ID NO: 16), DANA (SEQ ID NO: 17), EANA (SEQ ID NO: 18), NADA (SEQ ID NO: 19), NAQA (SEQ ID NO: 20), HAHA (SEQ ID NO: 21), RARA (SEQ ID NO: 22), LALA (SEQ ID NO: 23), HSHS (SEQ ID NO: 24), HTHT (SEQ ID NO: 25), LSLS (SEQ ID NO: 26), LTLT (SEQ ID NO: 27), RSRS (SEQ ID NO: 28), RTRT (SEQ ID NO: 29), HPHP (SEQ ID NO: 30), HGHG (SEQ ID NO: 31), LPLP (SEQ ID NO: 32), LGLG (SEQ ID NO: 33), DSDS (SEQ ID NO: 34), ESES (SEQ ID NO: 35), DSES (SEQ ID NO: 36), ESDS (SEQ ID NO: 37), NSNS (SEQ ID NO: 38), QSQS (SEQ ID NO: 39), DSNS (SEQ ID NO: 40), ESNS (SEQ ID NO: 41), NSDS (SEQ ID NO: 42), NSQS (SEQ ID NO: 43), DTDT (SEQ ID NO: 44), ETET (SEQ ID NO: 45), DTET (SEQ ID NO: 46), ETDT (SEQ ID NO: 47), NTNT (SEQ ID NO: 48), QTOT (SEQ ID NO: 49), DTNT (SEQ ID NO: 50), ETNT (SEQ ID NO: 51), NTDT (SEQ ID NO: 52), NTQT (SEQ ID NO: 53), DPDP (SEQ ID NO: 54), EPEP (SEQ ID NO: 55), DPEP (SEQ ID NO: 56), EPDP (SEQ ID NO: 57), NPNP (SEQ ID NO: 58), QPQP (SEQ ID NO: 59), DPNP (SEQ ID NO: 60), EPNP (SEQ ID NO: 61), NPDP (SEQ ID NO: 62), NPQP (SEQ ID NO: 63), DGDG (SEQ ID NO: 64), EGEG (SEQ ID NO: 65), DGEG (SEQ ID NO: 66), EGDG (SEQ ID NO: 67), NGNG (SEQ ID NO: 68), QGQG (SEQ ID NO: 69), DGNG (SEQ ID NO: 70), EGNG (SEQ ID NO: 71), NGDG (SEQ ID NO: 72), NGOG (SEQ ID NO: 73), HAHS (SEQ ID NO: 74), RARS (SEQ ID NO: 75), LALS (SEQ ID NO: 76), HSHT (SEQ ID NO: 77), HTHP (SEQ ID NO: 78), LSLT (SEQ ID NO: 79), LTLP (SEQ ID NO: 80), RSRT (SEQ ID NO: 81), RTRP (SEQ ID NO: 82), HPHG (SEQ ID NO: 83), HGHA (SEQ ID NO: 84), LPLA (SEQ ID NO: 85), LGLA (SEQ ID NO: 86), and combinations thereof, wherein X⁴ is any amino acid.

32-33. (canceled)

34. A method for treating a viral infection, optionally a coronavirus infection or an HIV infection, further optionally a SARS-CoV infection, a SARS-CoV-2 infection, and/or a porcine epidemic diarrhea virus (PEDV) infection, in a subject in need thereof, the method comprising administering to the subject a vaccine composition according to claim 20 in an amount and via a route sufficient to induce an anti-viral immune response in the subject.

35-36. (canceled)

37. The method of claim 33, wherein vaccine composition comprises and/or encodes:
(i) a coronavirus fusion peptide (FP) antigen, optionally a coronavirus FP antigen comprising an amino acid sequence selected from the group consisting of PSKPSKRSFIEDLLFNKVTLADAGF (SEQ ID NO: 2), PSKPSKRSFIEDLLFNKVTLADAG (SEQ ID NO: 1), SFIEDLLF (SEQ ID NO: 4, SFIEDLLFNKVTLADAGF (SEQ ID NO: 3), GRVVQKRSFIEDLLFNKVVTNGLG (SEQ ID NO: 5), and immunogenic fragments thereof; or
(ii) an HIV antigen comprising an amino acid sequence selected from the group consisting of AVGIGAVF (SEQ ID NO: 7), ALGIGAAF (SEQ ID NO: 8), AVGFGAAF (SEQ ID NO: 9), AAGFGAMF (SEQ ID NO: 10), and immunogenic fragments thereof; or
(iii) any combination thereof.

38. A composition comprising a modified bacterium or derivative thereof that expresses one or more tandem copies of a polypeptide of interest fused to a bacterial autotransporter β-barrel polypeptide, wherein:
(i) at least one copy of the one or more tandem copies of the polypeptide of interest is modified at or near its N-terminus to comprise at least one polar amino acid and/or an expression enhancement peptide comprising, consisting essentially of, or consisting of one or more copies of the amino acid sequence (X¹X²X³)ₙ, wherein each X¹ is independently selected from the group consisting of Asp (D), Asn (N), Glu (E), Gln (Q), His (H), Arg (R), and Lys (K); each X² is independently selected from the group consisting of Ala (A), Ser(S), Thr (T), Pro (P), Gly (G), Met (M), Leu (L), Ile (I), Val (V), and Cys (C); each X³ is absent in any given member of the one or more copies of the amino acid sequence (X¹X²X³)ₙ or, if present, is independently any amino acid; and n is at least 1; and
(ii) the polypeptide of interest is expressed on a surface of the modified bacterium or the derivative thereof.

39-52. (canceled)
